(19) United States
(12) Patent Application Publication
(10) Pub. No.: US 2025/0003008 A1
(43) Pub. Date: Jan. 2, 2025

(54) METHODS FOR TREATMENT OF CANCER

(71) Applicant: The Johns Hopkins University, Baltimore, MD (US)

(72) Inventors: Valsamo Anagnostou, Baltimore, MD (US); Noushin Niknafs Kermani, Baltimore, MD (US); Victor E. Velculescu, Glenwood, MD (US); Patrick Forde, Baltimore, MD (US)

(21) Appl. No.: 18/707,538

(22) PCT Filed: Nov. 7, 2022
(86) PCT No.: PCT/US2022/079403

§ 371 Date: May 3, 2024

Related U.S. Application Data
(60) Provisional application No. 63/276,525, filed on Nov. 5, 2021.

(51) Int. Cl. C12Q 1/6886

(52) U.S. Cl. CPC C12Q 1/6886; C12Q 2600/106; C12Q 2600/156

(57) ABSTRACT

In one aspect, methods are provided for assessing or treating a subject diagnosed with a tumor, comprising 1) obtaining a biological sample from the subject; 2) identifying germline or somatic mutations in the subject's genome; 3) identifying copy number profiles across the genome; 4) analyzing T cell receptor (TCR) clonotypes; and 5) identifying subjects responsive to chemotherapy, immunotherapy or combined chemotherapy and immunotherapy. In certain aspects, the identified subjects may be administered a chemotherapeutic, an immunotherapeutic or a chemotherapeutic and an immunotherapeutic, and, thereby treating the subject.

The present application claims the benefit of U.S. provisional application 63/276,525 filed Nov. 5, 2021, which is incorporated by referenced herein.

### STATEMENT REGARDING FEDERALLY SPONSORED RESEARCH FIELD

This invention was made with government support under grant W81XWH-20-1-0638 awarded by the U.S. Army. The government has certain rights in the invention.

### BACKGROUND

Since the 2011 approval of the prototypical ICI, the anti-CTLA-4 antibody ipilimumab, for the treatment of advanced melanoma, immune checkpoint inhibition has become a standard treatment option across solid tumors. As of early 2022, ICIs have been approved by the United States Food and Drug Administration (FDA) for the treatment of 17 distinct solid tumor histologies in addition to two tumor-agnostic indications for microsatellite instability-high tumors. PD-L1 expression and tumor mutation burden have been shown to in-part predict clinical responses to immune checkpoint blockade. Nevertheless, with the exception of mismatch repair deficient tumors, TMB has failed to consistently demonstrate clinical utility in predicting responses to cancer immunotherapy. Efforts to separate subsets of alterations that may predominantly drive an effective anti-tumor immune response have yet to reveal a universal genomic predictive biomarker.

Malignant pleural mesothelioma (MPM) affects more than 30,000 people worldwide each year and is fatal in nearly all cases¹. Exposure to asbestos and consequent chronic inflammation in the pleural cavity is responsible for the majority of cases with a typical disease latency of 30-40 years, especially in the context of co-occurring defects in DNA repair and germline cancer predisposition syndromes²⁻⁴. For over 15 years, cisplatin and pemetrexed combination chemotherapy was the only approved systemic therapy; this approval was based on a phase 3 study that showed an improvement in survival from 9.3 months with cisplatin alone to 12.1 months with the combination⁵. With the exception of bevacizumab (which has not achieved regulatory approval in the United States), adding novel agents to platinum doublet chemotherapy has not improved survival⁶⁻⁹. Recently, several phase 2 studies have reported on the efficacy of single agent PD-1 inhibitors in chemotherapy-pretreated MPM¹⁰⁻¹³.

### SUMMARY

We now provide new methods for treating subject suffering from a cancer, including a subject having one or more solid tumors.

In one aspect, methods are provided for assessing, monitoring, and/or treating mammals (e.g., humans) having, or suspected of having, cancer including solid tumors.

In a particular aspect, a sample (e.g., a blood sample) obtained from a mammal having one or more tumors can be assessed to determine.
1) whether the mammal (e.g. human) subject has a high number of mutations in haploid regions of the genome (which is predictive of chemotherapeutic and/or immunotherapeutic treatment being therapeutically effective against the mammal's tumor(s)); and/or
2) whether the mammal (e.g. human) subject has a high number of mutations in polyploid regions of the genome (which is predictive of chemotherapeutic and/or immunotherapeutic treatment being therapeutically effective against the mammal's tumor(s)); and/or
3) whether the mammal (e.g. human) subject has a high number of mutations in haploid and polyploid regions of the genome (which is predictive of chemotherapeutic and/or immunotherapeutic treatment being therapeutically effective against the mammal's tumor(s)).

Subjects having one or more tumors and that do not exhibit such high number of mutations in polyploid regions of the genome may not therapeutically benefit, or at may benefit less from such chemotherapeutic and/or immunotherapeutic treatment.

A high number of mutations in the haploid and/or polyploid regions of the genome may include a 3, 5, 10, 15, 20, 25, 30, 35, 40, 45, 50, 60, 70, 80, 90 or 100 percent or more increased number of mutations in such regions relative to a subject that would not be responsive to chemotherapeutic and/or immunotherapeutic treatment.

In one aspect, methods are provided for assessing and/or treating a subject diagnosed with a tumor, comprising: 1) obtaining a biological sample from the subject; 2) identifying germline or somatic mutations in the subject's genome; 3) analyzing T cell receptor (TCR) clonotypes; 4) determining the number of mutations in haploid and/or polyploid regions of the subject's genome; and 5) selecting the subject as being responsive to chemotherapy, immunotherapy or combined chemotherapy and immunotherapy treatment when the subject is determined to have a high number of mutations in haploid and/or polyploid regions of the subject's genome.

In certain aspects, a subject is identified as having mutations in tumor suppressor genes, DNA repair genes, chromatin regulating genes and/or defects in homologous recombination are predictive of responsiveness to chemotherapeutic and/or immunotherapeutic treatment.

In particular aspects, subjects are identified as having a high number of mutations in haploid regions of the genome and those identified subjects are identified or selected or assessed as being favorably responsive to chemotherapeutic and/or immunotherapeutic treatment.

In additional particular aspects, subjects are identified as having a high number of mutations in polyploid regions of the genome and those identified subjects are identified or selected or assessed as being favorably responsive to chemotherapeutic and/or immunotherapeutic treatment.

In further particular aspects, subjects are identified as having a high number of mutations in haploid and polyploid regions of the genome and those identified subjects are identified or selected or assessed as being favorably responsive to chemotherapeutic and/or immunotherapeutic treatment.

Again, a high number of mutations in the haploid and/or polyploid regions of the genome may include a 3, 5, 10, 15, 20, 25, 30, 35, 40, 45, 50, 60, 70, 80, 90 or 100 percent or more increased number of mutations in such regions relative to a subject that would not be responsive to chemotherapeutic and/or immunotherapeutic treatment.

In certain embodiments, the tumor comprises a low tumor mutation burden (TMB-L), a medium tumor mutation burden (TMB-M) or a high tumor mutation burden (TMB-H). Suitably, a high tumor mutational burden comprises 10 or more non-synonymous somatic mutations per megabase; a medium tumor mutational burden comprises between 2 to less than 10 non-synonymous somatic mutations per megabase; and a low tumor mutation burden comprises less than 2 non-synonymous somatic mutations per megabase.

In certain aspects, the tumor comprises a low tumor mutation burden.

The nonsynonymous sequence alterations suitably encode one or more immunogenic neoantigens, for example HLA class I and HLA class II restricted neoantigens.

In certain aspects, detection of an enrichment of HLA class I and HLA class II restricted neoantigens as compared to a control, is predictive of the subject's responsiveness to the immunotherapy.

In certain aspects, detection of a less clonal T cell receptor repertoire is predictive of the subject's responsiveness to the immunotherapy.

In certain aspects, one or more germline mutations are detected in one or more cancer susceptibility genes. For example, the one or more cancer susceptibility genes may comprise BAP1, MLH1, MLH3, BRCA1/2, BLM or combinations thereof.

In certain aspects, the one or more tumor suppressor genes comprise BAP1, CDKN2A. NF2, SETD2, PBRM1, TP53 or combinations thereof.

In certain aspects, the one or more chromatin genes comprise mutations in members of a SWI/SNF chromatin remodeling complex. A preferred SWI/SNF chromatin remodeling complex may comprise for example ARID1A and ARID1B genes.

In certain aspects, the present methods may further comprise detecting mutations in genes KDM3B and KDM4C encoding histone demethylases and in KMT2C gene encoding methyltransferases.

In certain aspects, subjects identified as having a high degree of tumor aneuploidy and genome-wide copy number breakpoints are predictive of responsiveness to combined chemotherapeutic and/or immunotherapeutic treatment.

In certain aspects, subjects identified as having defective homologous recombination are predictive of responsiveness to combined chemotherapy and immunotherapy.

In certain aspects, detection of mutation signatures is indicative of an apolipoprotein B mRNA editing catalytic polypeptide-like (APOBEC) mutagenesis are predictive of non-responsiveness to combined chemotherapy and immunotherapy.

In certain aspects, the subject has cancer including without limitation, colorectal cancer, as well as, for example, leukemias, e.g., acute myelogenous leukemia (AML), chronic myelogenous leukemia (CML), acute lymphocytic leukemia (ALL), and chronic lymphocytic leukemia, AIDS related cancers such as Kaposi's sarcoma; breast cancers; bone cancers such as Osteosarcoma, Chondrosarcomas, Ewing's sarcoma, Fibrosarcomas, Giant cell tumors, Adamantinomas, and Chordomas; Brain cancers such as Meningiomas, Glioblastomas, Lower-Grade Astrocytomas, Oligodendrocytomas, Pituitary Tumors, Schwannomas, and Metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, lung cancer, bladder cancer, prostate cancer, lung cancer (including non-small cell lung carcinoma), pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers. In certain aspects, the subject has a solid tumor. In additional aspects, the subject has non-small cell lung cancer. In further aspects, the subject has head and neck cancer. In further aspects, the subject has melanoma. In certain aspects, the subject has a mesothelioma, such as malignant pleural mesothelioma (MPM). In yet further aspects, the subject has an epithelioid MPM.

Any of a variety of chemotherapeutic agents may be administered to a subject in accordance with the present methods including for example: cisplatin (CDDP), carboplatin, bevacizumab, procarbazine, mechlorethamine, cyclophosphamide, camptothecin, ifosfamide, melphalan, imatinib mesylate, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein transferase inhibitors, transplatinum, 5-fluorouracil, vincristine, vinblastine, methotrexate, temazolomide, platinum or any analog or derivative variants or combinations thereof.

Similarly, any of a variety of immunotherapeutic agents may be administered to a subject in accordance with the present methods including for example: a checkpoint inhibitor, cytokines, antibodies, adoptive cell therapy, co-stimulatory receptor agonist, a stimulator of innate immune cells, an activator of innate immunity, chimeric antigen receptor T cells (CAR-T), CAR-NK cells, a toll like receptor (TLR) agonist and combinations thereof. In certain aspects the administered immunotherapeutic agent may comprise: durvalumab, nivolumab, or atezolizuma and combinations thereof.

In certain aspects, a method of identifying and distinguishing single-copy, multi-copy and persistent tumor mutations (pTMB) in a biological sample comprises performing a genome-wide analysis of sequence coverage distribution and b-allele frequency of heterozygous single nucleotide polymorphisms (SNPs) for determining purity and segmental tumor and normal copy numbers from either whole genome, whole exome or targeted panel next-generation sequencing; calculating the expected variant allele fraction for a mutation at a cellular fraction with mutant copies per cancer cell and calculating mutation clonality for each cancer cell in the biological sample; assigning minor and major copy numbers to mutated loci and classifying mutations as single-copy, multi-copy or persistent tumor mutations; and identifying and distinguishing single-copy, multi-copy and persistent tumor mutations. In certain embodiments, wherein mutation multiplicity (number of mutated copies per cell) and cancer cell fraction (proportion of cancer cells harboring the mutation) are calculated based on the mutant read count, total coverage, tumor purity, and major and minor allele-specific copy number in the tumor and normal counterpart for each mutation. In certain embodiment, mutations in regions of the genome with a single copy are classified as single copy mutations. In certain embodiments, mutations present in more than one copy per cancer cell are classified as multi-copy mutations. In certain embodiments, the persistent tumor mutations are defined as the number of mutations classified as either single-copy or multi-copy mutations. In certain aspects, a method of predicting response to therapy, e.g. immunotherapy, chemotherapy, comprises computing persistent tumor mutation burden by whole exome or targeted next generation sequencing.

In certain aspects, a method of assessing differential potential of persistent mutations in predicting cancer outcome compared to tumor mutation burden (TMB), comprises defining a number of loss-prone mutations in each tumor sample. In certain embodiments, the number of loss-prone mutations in each tumor sample is defined as the difference between the total number of mutations assessed and the number of persistent mutations.

In further aspects, methods and systems are provided that include assessing the number of persistent mutations in tumors of a subject to predict clinical response from immunotherapy containing regimens, in tumors with high tumor mutation burden for which tumor mutation burden failed to do so.

In yet further aspects, methods and systems are provided that include assessing the number of persistent mutations in tumors of subject to identify tumors that can be prospectively selected and treated with immunotherapy containing regimens, including in the setting of an interventional biomarker-directed clinical trial.

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within S-fold, and also within 2-fold, of a value. Where particular values are described in the application and claims, unless otherwise stated the term “about” meaning within an acceptable error range for the particular value should be assumed.

The term “cancer” as used herein is meant, a disease, condition, trait, genotype or phenotype characterized by unregulated cell growth or replication as is known in the art; including lung cancer (including non-small cell lung carcinoma), gastric cancer, colorectal cancer, as well as, for example, leukemias, e.g., acute myelogenous leukemia (AML), chronic myelogenous leukemia (CML), acute lymphocytic leukemia (ALL), and chronic lymphocytic leukemia, AIDS related cancers such as Kaposi's sarcoma; breast cancers; bone cancers such as Osteosarcoma, Chondrosarcomas, Ewing's sarcoma, Fibrosarcomas, Giant cell tumors, Adamantinomas, and Chordomas; Brain cancers such as Meningiomas, Glioblastomas, Lower-Grade Astrocytomas, Oligodendrocytomas, Pituitary Tumors, Schwannomas, and Metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, bladder cancer, prostate cancer, pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, and multidrug resistant cancers.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

An “effective amount” as used herein, means an amount which provides a therapeutic or prophylactic benefit.

“Optional” or “optionally” means that the subsequently described event or circumstance can or cannot occur, and that the description includes instances where the event or circumstance occurs and instances where it does not.

As used in this specification and the appended claims, the term “or” is generally employed in its sense including “and/or” unless the content clearly dictates otherwise.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some embodiments, the methods of the invention find use in experimental animals, in veterinary application, and in the development of animal models for disease, including, but not limited to, rodents including mice, rats, and hamsters, and primates.

The terms “sample,” “patient sample,” “biological sample,” and the like, encompass a variety of sample types obtained from a patient, individual, or subject and can be used in a diagnostic, prognostic and/or monitoring assay. The patient sample may be obtained from a healthy subject, a diseased patient, or a patient with lung cancer. In certain embodiments, a sample that is “provided” can be obtained by the person (or machine) conducting the assay, or it can have been obtained by another, and transferred to the person (or machine) carrying out the assay. Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, peripheral blood, serum, plasma, cord blood, amniotic fluid, cerebrospinal fluid, urine, saliva, stool and synovial fluid), solid tissue samples such as a biopsy specimen or tissue cultures or cells derived therefrom and the progeny thereof. In certain embodiment, a sample comprises cerebrospinal fluid. In a specific embodiment, a sample comprises a blood sample. In another embodiment, a sample comprises a plasma sample. In yet another embodiment, a serum sample is used. The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the term “tumor” means a mass of transformed cells that are characterized by neoplastic uncontrolled cell multiplication and at least in part, by containing angiogenic vasculature. The abnormal neoplastic cell growth is rapid and continues even after the stimuli that initiated the new growth has ceased. The term “tumor” is used broadly to include the tumor parenchymal cells as well as the supporting stroma, including the angiogenic blood vessels that infiltrate the tumor parenchymal cell mass. Although a tumor generally is a malignant tumor, i.e., a cancer having the ability to metastasize (i.e. a metastatic tumor), a tumor also can be nonmalignant (i.e. non-metastatic tumor). Tumors are hallmarks of cancer, a neoplastic disease the natural course of which is fatal. Cancer cells exhibit the properties of invasion and metastasis and are highly anaplastic.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1. (includes FIGS. 1a-1f) Outcomes with chemo-immunotherapy in unresectable malignant pleural mesothelioma. (a) Kaplan-Meier curve of overall survival in patients treated with durvalumab and platinum plus pemetrexed (n=55). One-sided p-value based on Wald test for the log failure rate parameter is p=0.0014, indicating significantly longer OS than the historic control of 12 months (b) Kaplan-Meier curve of progression-free survival in patients treated with durvalumab and platinum plus pemetrexed (n=55) (c) Waterfall plot of best change in target lesions to treatment by histologic subtype, based on maximal percentage of tumor reduction from baseline (n=53 patients). Two patients without follow-up measurements in targeted lesions (one with best response unevaluable, the other with best response PD) were excluded. (d) Spider plot of change in target lesions over time (n=53 patients); notably, four patients had continued response or stable disease at the time of analysis. Two patients without follow-up measurements in targeted lesions (one with best response unevaluable, the other with best response PD) were excluded. (e) Kaplan-Meier curves of OS according to histology; two sided p value with significance level set at 0.05. (f) Kaplan-Meier curves of PFS according to histology; two sided p value with significance level set at 0.05. Abbreviations: Epi; epithelioid, MPM; malignant pleural mesothelioma, OS; overall survival, PFS; progression-free survival, CI; confidence interval, PR; partial response, SD; stable disease, PD; progressive disease.

FIG. 2 Genomic landscape of chemo-immunotherapy treated mesotheliomas. MPMs of patients with a radiographic response harbored a higher number of nonsynonymous missense sequence mutations (n=40 MPM tumors, on average 23 vs 18 mutations per exome for responders and non-responders respectively, Mann Whitney p=0.086). Epithelioid MPMs responding to therapy harbored a higher number of clonal missense mutations (n=29 tumors, Mann Whitney p=0.051 and 0.025 for missense mutation load and clonal mutations respectively); the numbers of subclonal mutations are shown as yellow inserts. Recurring inactivating alterations in BAP1, CDKN2A, NF2, TP53, SETD2 and PBRM1 did not differentially cluster with regards to therapeutic responses. Similarly, somatic BAP1 sequence alterations and CDKN2A homozygous deletions were detected in 32.5% (13 out of 40) and 30% (12 out of 40) of MPMs, without a notable enrichment with respect to therapeutic response. Specific genotypes were associated with exceptional therapeutic outcome (PFS≥12 months and/or OS≥24 months); patient 178 harbored tumor biallellic inactivation of NF2 and the histone methyltransferase SETD2, patient 926 harbored tumor biallellic inactivation of BAP1 and patient 361 harbored tumor homozygous deletions in BAP1 and in the SWI/SNF nucleosome remodeling gene PBRM1. An enrichment in mutations in chromatin regulating genes was observed for patients achieving an overall survival ≥12 months (Fisher's exact p=0.063). We identified a higher contribution of an HRD mutation signature in responsive tumors (n=40 patients, average HRD contribution 9.1% vs 1.6% in responders and non-responders respectively, Mann Whitney p=0.043). Conversely, an APOBEC mutation signature was found to be more enriched in MPM and epithelioid MPM of non-responders (Mann Whitney p=0.058 and p=0.031 respectively). Mutations were characterized by consequence (missense, frameshift, nonsense, splice site) and recurrence (hotspots, depicted as solid circles) and loss of the wild type allele was considered in case of truncating mutations (biallellic inactivation, marked with an “x”). Tumor samples from patients 329, 351, 629, and 923 were excluded from analyses of somatic alterations due to tumor tissue quality and are not shown here; these patients were included in the germline analyses with patient 629 harboring a deleterious mutation in BAP1.

FIG. 3 (includes FIGS. 3a-d) Impact of germline mutations in cancer susceptibility genes on outcome from combined immuno-chemotherapy. (a-b) Patients harboring known deleterious germline mutations in mesothelioma-predisposing genes (Methods) had a longer overall survival (log rank p=0.05) especially in the epithelioid MPM group (log rank p=0.032). (c-d) A focused analysis including deleterious germline mutations in BAP1, BRCA2, MSH6 and BLM, all genes involved in DNA damage repair, showed the same trends towards a longer overall survival for patients harboring germline mutations in DDR genes (log rank p=0.12 for all patients and log rank p=0.082 for epithelioid patients). All p values are two-sided. Abbreviations: MPM; malignant pleural mesothelioma, DDR; DNA damage repair.

FIG. 4. (includes FIGS. 4a-4d) Large-scale copy number analyses. (a) Genome-wide copy number analyses predominantly revealed genomic regions with copy number losses (shown in blue) and were used to determine the extent of copy number breakpoints and fraction of genome with complete allelic imbalance reflecting genomic instability and tumor aneuploidy. The relative copy ratio (log copy ratio) values quantifying the abundance of each genomic region compared to the average genome ploidy are shown per chromosome after correction for tumor purity. Red and blue shades indicate copy gains and losses, respectively, whereas white marks copy neutral regions. A homologous recombination deficiency score was computed taking into account telomeric allelic imbalance, LOH and large-scale state transitions. Three extreme cases of LOH were noted, with a copy number pattern that was suggestive of genome near-haploidization; these patients had an overall survival ≥12 months after chemo-immunotherapy. (b-c) A higher number of copy number breakpoints and a higher homologous recombination deficiency score distinguished epithelioid MPM from patients that achieved an overall survival ≥12 months (n=28 epithelioid MPM tumors, Mann Whitney p=0.0.05 and p=0.014 respectively). (d) Responding tumors harbored a higher number of mutations in single copy regions of the genome, suggesting that these “difficult” to eliminate alterations and associated neoantigens may be important drivers of the antitumor immune response (n=40 MPM tumors, Mann Whitney p=0.027). The center line in the boxplots represents the median, the upper limit of the boxplots represents the third quantile (75th percentile), the lower limit of the boxplots represents the first quantile (25th percentile), the upper whiskers is the maximum value of the data that is within 1.5 times the interquartile range over the 75th percentile, and the lower whisker is the minimum value of the data that is within 1.5 times the interquartile range under the 25th percentile. All p values are two-sided. Abbreviations: MPM; malignant pleural mesothelioma, OS; overall survival, BOR; best overall response, HRD; homologous recombination deficiency, CN; copy number, Allelic Imbal. Frac.; fraction of genome with allelic imbalance, GNH; genome near haploidization, CR: complete response, PR; partial response, SD; stable disease, PD; progressive disease.

FIG. 5 (includes FIGS. 5a-5d). Baseline TCR repertoire characteristics and dynamic changes at the time of therapeutic resistance. (a) The intratumoral T cell repertoire was interrogated by TCR Vβ sequencing, clonality of the TCR repertoire was computed and the representation of dominant clones (Methods) as a proportion of the whole TCR repertoire was determined. CD8+ T cell density and PD-L1 tumor proportion scores for each evaluable case are shown (missing cases are shown in gray). (b) These analyses revealed a less clonal TCR repertoire in tumors from patients achieving an overall survival ≥12 months (Mann Whitney p=0.018). (c) A higher representation of dominant clones was also detected in tumors from patients with an inferior overall survival (Man Whitney p=0.006). (d) Differential abundance analyses of 3 cases with available tumor samples prior to therapy initiation (295, 459 and 926) and at the time of acquired resistance revealed TCR clonotypic expansions (labeled as significant positive) and regressions (labeled as significant negative) shown for patient 926, that achieved a partial response and an overall survival of 27.8 months. Fold change of intra-tumoral TCR clones is plotted on the x axis (log scale) and the adjusted corresponding Mann Whitney p value is shown on the y axis (−log scale) of the volcano plot. All p values are two-sided. Abbreviations: OS; overall survival, BOR; best overall response, CR; complete response, PR; partial response, SD; stable disease, PD; progressive disease, NE; non-evaluable, Epi; epithelioid, Sarc; sarcomatoid, Biphas; biphasic, IHC; immunohistochemistry, TCR; T cell receptor, NS; non-significant, Neg; significantly negative (regressing TCR clones), Pos; significantly positive (expanding TCR clones).

FIG. 6. Pan-cancer analyses of copy number loss rates in euploid versus haploid regions of the genome.

FIG. 7. Survival analyses shows overall survival benefit of persistent mutation load-based stratification compared to traditional tumor mutation burden in non-small cell lung cancer and melanoma.

FIG. 8 (includes FIGS. 8A-8D). Persistent mutation burden more accurately predicts response to immune checkpoint blockade across tumor types and therapies.

FIG. 9 (includes FIGS. 9A-9D). Pan-cancer distribution of persistent mutation load. FIG. 9A: The background rate of genomic loss was quantified in 31 tumor types from TCGA (N=5,244). In all tumor types, the rate of loss was significantly higher in diploid (allele-specific copy numbers 1-1) vs haploid (single copy; allele-specific copy numbers 1-0) regions of the genome (Mann-Whitney U-test, p<0.05 for all tumor types except for UCS for which Mann Whitney p=0.067), supporting the notion that mutations that reside in haploid regions of the genome would less likely be lost. Boxplots depict the median value and hinges correspond to the first and third quartiles. The whiskers extend from the corresponding hinge to the furthest value within 1.5*the interquartile range from the hinge. FIG. 9B: An analysis of somatic copy number aberrations in 9,991 tumors across 31 tumor types from TCGA identified the fraction of the genome with a single copy present (total copy number of 1, blue) and with multiple copies of a parental haplotype (red). A differential enrichment pattern was noted, whereby cancers including UCS, BLCA, ACC, LUSC, LUAD, OV and SKCM had a higher fraction of the genome with multiple copies compared to CHOL, PAAD, MESO and KICH that showed a higher genome fraction in the only-copy state. Violin plots depict the distribution of fractions in each state, and the horizontal black segments indicate median values. Tumor types with predominance of single-copy genome fraction are marked in blue font. FIG. 9C: The prevalence of somatic mutations present in multiple copies per cell (multi-copy), and those present in haploid regions of the genome (only-copy) is depicted for 9,242 tumors, highlighting similar differential distributions per cancer type. FIG. 9D: The number of multi-copy and only-copy somatic mutations is shown against a background of the median TMB of the corresponding tumor type. Notably, the median TMB within a tumor type does not fully predict the abundance of multi-copy and only-copy mutations, and tumor types with very similar TMB may exhibit differences in multi-copy and only-copy mutation load (for instance UCS vs GBM and SARC).

FIG. 10 (includes FIGS. 10A-10C). Evaluation of the association between persistent mutation content and TMB. FIG. 10A: analysis of 9,242 tumors across 31 tumor types revealed a large variation in the correlations between TMB and pTMB (blue bars), which is not entirely explained by TMB alone (median TMB values within each tumor type are shown in the black trace). FIG. 10B: the tumor reclassification rate was calculated by applying a given quantile of TMB and pTMB and is shown for 31 tumor types within the TCGA cohort (light blue traces), with the median reclassification rate depicted in a dark blue line. In tumors such as UVM and UCEC, up to 40% of the samples could be differentially classified as pTMB-high vs pTMB-low using persistent mutations rather than the overall TMB value. FIG. 10C: The fraction of persistent mutations exhibited a variable degree of correlation with TMB in five ICB-treated cohorts (n=524) across four tumor types. In HNSCC (n=39), melanoma (n=202), and mesothelioma (n=40), no significant correlation was observed (Spearman rho=−0.083, rho=0.066, rho=0.065, p>0.1 for all non-parametric correlations), while a weak/moderate correlation was observed in NSCLC (NSCLC-Anagnostou, n=74, Spearman rho=0.26, p=0.028 and NSCLC-Shim n=169, Spearman rho=0.41, p=2.3e-08). Spearman's rank correlation coefficients and corresponding p values are shown in inserts in FIG. 10C.

FIG. 11 (includes FIGS. 11A-11G) pTMB is linked with therapeutic response with immune checkpoint blockade. FIG. 11A: A representative example of a patient with melanoma harboring an intermediate TMB (59% quantile) but high pTMB (81% quantile); the latter accurately reflecting a sustained response to immune checkpoint blockade. The outer ring depicts integer segmental total copy number profile, the middle ring indicates segments with loss of heterozygosity in black, while the genomic coordinates of somatic mutations and their mutant allele fraction are shown in the inner ring. Mutations are colored by their estimated multiplicity value, i.e. the number of mutant copies per cancer cell. FIG. 11B: The role of pTMB in differentiating responding (R) from non-responding (NR) tumors was evaluated in 524 patients with melanoma, NSCLC, mesothelioma and HNSCC who received immune checkpoint blockade. In the melanoma cohort (n=202; NR=115, R=87), pTMB distinguished responding (blue) from non-responding tumors (red; Mann-Whitney U-test, p=2.3e-06 for pTMB, p=6.0e-07 for clonal pTMB) more optimally compared to TMB (Mann Whitney U-test p=2.6e-05). Boxplots depict the median value and hinges correspond to the first and third quartiles. The whiskers extend from the corresponding hinge to the furthest value within 1.5*the interquartile range from the hinge. FIG. 11C: In HPV-negative HNSCC, we found a greater difference in pTMB of responding (blue) vs non-responding tumors (red) compared to TMB (n=39; NR=29, R=10; Mann Whitney U test p=0.046 for pTMB, p=0.064 for clonal pTMB, and p=0.091 for TMB). FIG. 11D: For mesotheliomas, given the predominance of copy number losses the persistent mutation burden shown was limited to mutations within single-copy regions of the genome. Similarly to the melanoma and HNSCC cohorts, pTMB outperformed TMB in prediction of response to durvalumab plus platinum-pemetrexed chemotherapy in patients with unresectable pleural mesotheliomas (n=40; NR=16, R=24; Mann Whitney U test p=0.032 for pTMB, p=0.045 for clonal pTMB, while non-significant for TMB). FIG. 11E-11F: In the NSCLC cohort, a higher pTMB differentiated responding (blue) from non-responding (red) tumors (NSCLC-Anagnostou: n=74; NR=41, R=33; Mann Whitney U test p=1.3e-04 for pTMB, p=1.0e-04 for clonal pTMB and p=4.3e-04 for TMB; NSCLC-Shim: n=169, NR=49, R=120; Mann Whitney U test p=1.9e-03 for pTMB, p=1.6e-03 for clonal pTMB and p=8.0e-03 for TMB). FIG. 11G: The significance of association for eight sequence mutation-based and two copy number-based features was compared to the significance of the association between TMB and therapeutic response in each cohort (log₁₀ of feature to TMB p-value ratios visualized). The fraction of genome with allelic imbalance is used as a quantitative metric of aneuploidy, while the number of mutations within TMB that are more prone to elimination during tumor evolution were considered independently, in the loss-prone category. For each cohort, the feature with the most significant association with therapeutic response is marked with a black “x” mark. P-value ratios were capped at a value of 1 to highlight features with more significant associations with therapeutic response compared to TMB (negative values in the log₁₀ scale). Darker shades of blue indicate more significant association, whereas darker shades of red represent less significant association. For all ICB cohorts, persistent mutations outperformed TMB in predicting response to ICB. Notably, the best performing feature in the NSCLC was the clonal persistent mutation burden (MW p=1.03e-04, p=1.60e-03 for NSCLC-Anagnostou and NSCLC-Shim respectively), while in melanoma the number of multi-copy persistent mutations (MW p=5.42e-07) and in mesothelioma the number of only-copy persistent mutations (MW=3.15e-02) better distinguished between responding and non-responding tumors. pTMB outperformed loss-prone mutation load in distinguishing responding from non-responding tumors (HNSCC; Mann Whitney U-test p=0.16 vs. p=0.05, melanoma; Mann Whitney U-test p=1.92e-03 vs p-2.25e-06, mesothelioma; Mann Whitney U-test p=0.09 vs p=0.03, NSCLC-Anagnostou; Mann Whitney U-test p=1.03e-02 vs p=1.26e-04, NSCLC-Shim; Mann Whitney U-test p=3.20e-02 vs p=1.87e-03). Tumor aneuploidy alone failed to predict response to ICB in all cohorts assessed (MW p=0.35, p=0.73, p=0.35, p=0.07, p=0.50 for the NSCLC-Anagnostou, NSCLC-Shim, aggregated melanoma, mesothelioma and HNSCC respectively). Similarly, whole genome doubling events were not associated with therapeutic response (Fisher's exact p=0.43, p=0.73, p=0.11, p=0.23, p=0.48 for the NSCLC-Anagnostou, NSCLC-Shim, aggregated melanoma, mesothelioma and HNSCC respectively).

FIG. 12 (includes FIGS. 12A-12B). Persistent mutations are retained during cancer evolution under selective pressure of ICB. FIG. 12A: The presence of loss-prone, multi-copy, and only-copy mutations identified in pre-ICB treatment NSCLC tumors was evaluated post-ICB therapy in tumors from eight patients with NSCLC and durable clinical benefit from ICB. Serial tumor samples were acquired with a minimum time difference of 6 months between biopsies and were analyzed by means of whole exome sequencing. A marked difference in the frequency of loss between clonal persistent and loss-prone mutation sets was observed, with an odds ratio of 61.46 (p<2.2e-16). Across 16 serially biopsied tumors from 8 patients, a total of 363 out of 2836 clonal mutations that were detected in the baseline tumor were lost in the descendent tumor. Of these, the vast majority were clonal loss-prone mutations (358 out of 363, 98.6%). In 6 out of 8 patients analyzed, no clonal persistent mutation was lost in the descendent tumor, and of the two remaining patients, each had two clonal multi-copy mutations that were not detected in the descendent tumor, suggesting an extremely low rate of loss in this mutation category (clonal multi-copy mutations: 4 out of 1031 lost, 0.4% loss frequency, clonal only-copy mutations: 1 out of 117 lost, 0.9% loss frequency). FIG. 12B: While the rate of loss frequency was slightly higher for subclonal persistent mutations compared to subclonal mutations in the loss-prone category, this did not reach statistical significance (odds ratio=1.24, p=0.44). Notably, the loss frequency for subclonal multi-copy mutations was 9.3% compared to 14.8% for subclonal loss-prone mutations, again potentially indicating differential selection pressures for these alterations.

FIG. 13 (includes FIGS. 13A-13H) pTMB is associated with an inflamed tumor microenvironment in ICB treated melanomas. FIG. 13A: Gene set enrichment analysis leveraging transcriptomic profiles from RNA sequencing revealed a marked enrichment in interferon-γ response and adaptive immunity gene sets in ICB-treated melanomas with high vs low pTMB, assessed prior to immunotherapy initiation (dark red bars). In contrast, a significantly lower enrichment in inflammatory gene sets was observed in the TME of tumors stratified by their overall TMB (light red bars). The T Cell Inflamed GEP gene set was derived from Cristescu et al., Science, 2018 and the Inflammatory Response gene set was derived by Ayers et al., J Clin Invest, 2017, while the remainder of gene sets were included in the Molecular Signatures Database (Methods). A prominent upregulation of interferon gamma (FIG. 13B) and inflammatory response (FIG. 13C) related gene expression programs was noted in the TME of pTMB-high melanomas Quantile-quantile plots were generated to visually compare the ranks of genes in the pathway to ranks that were sampled from a discrete uniform distribution. FDR adjusted p values for gene set differential expression are provided for comparison of pTMB/TMB-high vs low groups. (FIG. 13D) pTMB counteracts the negative impact of aneuploidy on cytolytic activity and response to immune checkpoint blockade (GZMB, IFNG, PRF1: BpTMB=0.5, P-value<3e-03; βaneuploidy in [−0.09 , −0.03], P-value>0.05. ICB response, βpTMB=1.8, P-value=4.0e-03; βaneuploidy=−0.56, P-value>0.05). (FIGS. 13E-13H) A greater difference in cytolytic activity was observed between tumors of high versus low pTMB, compared to TMB and cytolytic activity (GZMB: TMB p=0.02, pTMB p=6.1e-03, aneuploidy p>0.05; IFNG: TMB p=0.01, pTMB p=5.6e-03, aneuploidy p>0.05; PRF1: TMB p=0.03, pTMB p=5.1e-03, aneuploidy p>0.05), while no significant difference in relative abundance of CD8 T cells was observed for TMB, pTMB or aneuploidy. Abbreviations: HM; Hallmark, KG; KEGG, RT; Reactome, Cyt; Cytokine, Rec; Receptor, EMT; Epithelial-mesenchymal transition, Med; mediated, FC; fold change, NS; non-significant.

FIG. 14 (includes FIGS. 14a-14c) show the association of intra-tumor clonal heterogeneity with persistent mutations. The correlation between the fraction of clonal mutations in each tumor sample (intra-tumoral clonal heterogeneity) and persistent mutations was assessed in the five cohorts treated with ICB (n=524). (a) A moderate degree of correlation between the number of multi-copy mutations and decreased tumor clonal heterogeneity was only observed in melanoma. (b) Higher clonality tumors tended to have a lower number of only-copy mutations in the melanoma and NSCLC-Shim cohorts (Spearman p=−0.15 and p≤0.05), but no such difference was observed in the HNSCC, mesothelioma, and NSCLC-Anagnostou cohorts. (p>0.16). (c) A significant correlation between pTMB and lower intra-tumoral clonal heterogeneity was observed in the melanoma cohort (Spearman p=0.33, p=1.7e-06). Spearman's rank correlation coefficients are shown, each tumor sample represents a point and points are color coded based on tumor response on ICB.

FIG. 15 demonstrates the contribution of whole-genome doubling to acquisition of persistent mutations. TMB, the number of persistent mutations, mutations present in multiple copies per cell (multi-copy), mutations in single-copy regions (only-copy) of the genome were compared between tumors with and without evidence of whole genome doubling (WGD) across the five ICB cohorts (n=524). In all cohorts analyzed, tumors with WGD harbored a significantly higher number of multi-copy mutations (HNSCC, p=1.6e-05, melanoma; p=3.14e-14, mesothelioma; p=8.24e-06, NSCLC-Anagnostou; p=6.82e-09, NSCLC-Shim; p=9.3e-17) but a lower number of only-copy mutations (HNSCC; p=5.23e-04, melanoma; p=2.33e-19, mesothelioma; p=6.13e-04, NSCLC-Anagnostou; p=1.21e-03, NSCLC-Shim; p=3.98e-09). HNSCC, melanoma, and mesothelioma tumors with and without WGD had similar levels of TMB (HNSCC; p=0.58, melanoma; p=0.76, mesothelioma; p=0.45), while in the NSCLC-Shim cohort tumors with WGD also harbored a higher TMB (p=5.91e-04). Tumors with WGD had significantly higher pTMB in melanoma, mesothelioma, and NSCLC, with a similar trend observed in HNSCC (HNSCC; p=0.070, melanoma; p=1.37e-06, mesothelioma; p=1.84e-05, NSCLC-Anagnostou; p=1.24e-05, NSCLC-Shim; p=2.25e-08). Boxplots depict the median value and hinges correspond to the first and third quartiles. The whiskers extend from the corresponding hinge to the furthest value within 1.5*the interquartile range from the hinge. Mann-Whitney U-test was used to compare values in tumors with and without WGD.

FIG. 16 shows the expression analyses of early stage melanomas in the TCGA cohort suggests a depletion of inflammatory pathways in high risk tumors as predicted by clonal pTMB. Gene set enrichment analyses of clonal pTMB-high risk (n=32) vs. clonal pTMB-low risk (n=63) melanomas highlight a significant under-representation of interferon-γ and inflammatory response gene sets in the microenvironment of tumors with pTMB-informed high vs low risk. In contrast, this pattern was not observed in tumors with TMB-informed high vs low risk. Pathways with a minimum adjusted p-value of 1e-05 in clonal pTMB high vs low risk comparison are shown. The T Cell Inflamed GEP gene set was derived from Cristescu et al., Science, 2018 and the Inflammatory Response gene set was derived by Ayers et al., J Clin Invest, 2017, while the remainder of gene sets were included in the Molecular Signatures Database (Methods). Abbreviations; HM: Hallmark, KG: KEGG, RT: Reactome, Cyt: Cytokine, Rec: Receptor, Med: Mediated.

FIG. 17 shows the association of pTMB with abundance of immune cell subpopulations estimated by transcriptomic analysis of baseline and on-immunotherapy melanomas. CIBERSORT was used for deconvolution of RNA sequencing data and computation of the abundance of key immune cell subsets in the tumor microenvironment. Positive correlations were observed between pTMB CD4 and CD8 T cells as well as proinflammatory macrophages M1, while an anti-correlation was observed between pTMB and M2 macrophages. The ratio of M1 to M2 macrophages in baseline (n=38, Spearman's ρ=0.36) and on-therapy tumors (n=31, Spearman's ρ=0.28) was positively correlated with pTMB.

FIG. 18 (includes FIGS. 18a-18d) shows the association of pTMB with cytolytic activity in melanoma. Expression of a selected set of genes indicative of cytolytic activity in TCGA melanoma tumors (n=95). (a) No significant difference in expression was found in the TME of tumors in the top (H, n=32) and bottom (L, n=32) tertiles of TMB. (b) A higher level of cytolytic activity was observed in the TME of tumors with high (H, n=32) vs low pTMB (L, n=32), as indicated by the higher expression of GZMK, IFNG, NKG7, and PRF1 (MW U-test p=0.021, 0.019, 0.04, and 0.019, respectively). (c) Tumor aneuploidy was not found to be a strong predictor of cytolytic activity in the set of analyzed tumors (MW U-test p>0.05). Boxplots depict the median value and hinges correspond to the first and third quartiles. The whiskers extend from the corresponding hinge to the furthest value within 1.5*the interquartile range from the hinge. (d) pTMB predicted cytolytic activity in the TME more accurately compared to TMB and aneuploidy. Phred-scaled Mann Whitney U-test p-values are visualized. The annotated signs indicate the direction of association with cytolytic activity; i.e. a higher level expression in the top tertile group is marked as positive.

FIG. 19 demonstrates the effects of pTMB and aneuploidy on cytolytic activity. Multivariate modeling of gene markers of cytolytic activity in the TME of TCGA melanoma tumors (n=95) showed that high pTMB counteracted the negative (but non-significant) impact of aneuploidy and was positively correlated with cytolytic activity. A high value of pTMB and a low level of aneuploidy predicted higher expression of GZMK and IFNG (GZMK: β_{pTMB}=0.24, p=0.03; β_{aneuploidy}=−0.06, p>0.05. IFNG: β_{pTMB}=0.23, p=0.03; β_{aneuploidy}=−0.12, p >0.05. CD8.rel: relative abundance of CD8 cells as estimated by CIBERSORT.

FIG. 20 is a schematic representation of the persistent mutation hypothesis. We hypothesized that tumors with a higher frequency of sequence alterations in either haploid regions or in multiple copies would be intrinsically incapable of escaping immune recognition in the setting of immunotherapy, as these alterations would continuously render them visible to the immune system, resulting in sustained tumor rejection. These two distinct genomic mechanisms produce a common feature that we term “persistent mutations”, a key driver of continued immunologic tumor control.

FIG. 21 (includes FIGS. 21a-21b) shows the interaction of TMB and fraction of mutations in single- and multi-copy regions across 31 cancer types. A combined analysis of TMB ranks in relation to the fraction of mutations in multi-copy (a) and only-copy (b) regions of the genome revealed a complex landscape, where a wide range of multi-copy or single-copy mutation fraction was observed at any given TMB value in 9,256 tumors across 31 cancer types from TCGA. These findings highlight tumors with a low or intermediate TMB that at the same time harbor a large fraction of mutations in multi-copy or single-copy states, therefore suggesting the independent nature of these features. A weak correlation was noted between TMB rank and fraction of mutations in multi-copy regions of the genome for BLCA (Pearson's R=0.26), KICH (Pearson's R=0.45), LUAD (Pearson's R=0.34), LUSC (Pearson's R=0.21), SARC (Pearson's R=0.24) while these features were weakly anti-correlated in COAD and UCEC (Pearson's R=−0.23 and −0.35 respectively). TMB was weakly anti-correlated with the fraction of mutations in haploid regions for COAD, STAD and UCEC (Pearson's R=−0.22, −0.25 and −0.25 respectively). The heatmaps indicate the distribution of tumors within a tumor type, and each black dot represents an analyzed tumor sample; raw values are transformed to ranks for more succinct visualization. Abbreviations: ACC; Adrenocortical carcinoma, BLCA; Bladder Urothelial Carcinoma, BRCA; Breast invasive carcinoma, CESC; Cervical squamous cell carcinoma and endocervical adenocarcinoma, CHOL; Cholangiocarcinoma, COAD; Colon adenocarcinoma, DLBCL; Lymphoid Neoplasm Diffuse Large B-cell Lymphoma, ESCA; Esophageal carcinoma, GBM; Glioblastoma multiforme, HNSC; Head and Neck squamous cell carcinoma, KICH; Kidney Chromophobe, KIRC; Kidney renal clear cell carcinoma, LAML; acute myeloid leukemia, LGG; Brain Lower Grade Glioma, LIHC; Liver hepatocellular carcinoma, LUAD; Lung adenocarcinoma, LUSC; Lung squamous cell carcinoma, MESO; Mesothelioma, OV; Ovarian serous cystadenocarcinoma, PAAD; Pancreatic adenocarcinoma, PCPG; Pheochromocytoma and Paraganglioma, PRAD; Prostate adenocarcinoma, READ; Rectum adenocarcinoma, SRC; Sarcoma, SKCM; Skin Cutaneous Melanoma, STAD; Stomach adenocarcinoma, TGCT; Testicular Germ Cell Tumors, THCA; Thyroid carcinoma, THYM; Thymoma, UCEC; Uterine Corpus Endometrial Carcinoma, UCS; Uterine Carcinosarcoma, UVM; Uveal Melanoma.

FIG. 22 demonstrates the tumor re-classification by pTMB vs TMB. A series of quantile values going from 5% to 95% in 5% increments were used to extract samples with high and low values for each predictor variable in each tumor type. For each quantile value, the re-classification rate per samples within a cancer type group. By definition, sample re-classification rate starts close to 0 at the lower end of quantile thresholds (where almost all samples are labeled as high regardless of the metric used) and returns to 0 at the higher end of quantile thresholds (where the great majority of samples are labeled as low irrespective of the metric used). In the intermediate range, re-classification rates as high as 50% were observed. Abbreviations: ACC; Adrenocortical carcinoma, BLCA; Bladder Urothelial Carcinoma, BRCA; Breast invasive carcinoma, CESC; Cervical squamous cell carcinoma and endocervical adenocarcinoma, CHOL; Cholangiocarcinoma, COAD; Colon adenocarcinoma, DLBCL; Lymphoid Neoplasm Diffuse Large B-cell Lymphoma, ESCA; Esophageal carcinoma, GBM; Glioblastoma multiforme, HNSC; Head and Neck squamous cell carcinoma, KICH; Kidney Chromophobe, KIRC, Kidney renal clear cell carcinoma, LAML; acute myeloid leukemia, LGG; Brain Lower Grade Glioma, LIHC; Liver hepatocellular carcinoma, LUAD; Lung adenocarcinoma, LUSC; Lung squamous cell carcinoma, MESO; Mesothelioma, OV; Ovarian serous cystadenocarcinoma, PAAD; Pancreatic adenocarcinoma, PCPG; Pheochromocytoma and Paraganglioma, PRAD; Prostate adenocarcinoma, READ; Rectum adenocarcinoma, SRC; Sarcoma, SKCM; Skin Cutaneous Melanoma, STAD; Stomach adenocarcinoma, TGCT; Testicular Germ Cell Tumors, THCA; Thyroid carcinoma, THYM; Thymoma, UCEC; Uterine Corpus Endometrial Carcinoma, UCS; Uterine Carcinosarcoma, UVM; Uveal Melanoma.

FIG. 23 (includes FIGS. 23a and 23b) shows the clonal architecture of persistent mutations. (a) We computed the fraction of clonal mutations within loss-prone, multi-copy, only-copy, and persistent mutations sets. In the HNSCC and melanoma cohorts, multi-copy mutations were more clonal compared to loss-prone mutations (HNSCC; p=0.01, melanoma; p=3.8e-14), but no such difference was present in the mesothelioma (p=0.66) and NSCLC cohorts (NSCLC-Anagnostou: p=0.59, NSCLC-Shim; p=0.40). Only-copy mutations had similar clonal fractions as loss-prone mutations (p>0.11) in all five cohorts. When multi-copy and only-copy mutations were considered together, we did not identify a significant difference in clonal fractions between persistent and loss-prone mutations in the HNSCC (p=0.59), mesothelioma (p=0.65), and NSCLC cohorts (NSCLC-Anagnostou; p=0.51, NSCLC-Shim; p=0.20). Persistent mutations tended to be more clonal in the melanoma cohort (p=8.80e-03) Boxplots depict the median value and hinges correspond to the first and third quartiles. The whiskers extend from the corresponding hinge to the furthest value within 1.5*the interquartile range from the hinge. Mann-Whitney U-test was used to compare values in across mutation classes. (b) A weak to moderate degree of correlation was observed between the fraction of clonal mutations and the number of persistent mutations (Spearman p range: −0.11-0.59) or multi-copy mutations (Spearman p range: 0.01-0.60) across the 31 TCGA tumor types analyzed. Notably, the number of only-copy mutations was anti-correlated with the fraction of clonal mutations in the TCGA dataset (Spearman p range: −0.57, 0.07). Spearman's rank correlation coefficients are depicted in the heatmap. Abbreviations: ACC; Adrenocortical carcinoma, BLCA; Bladder Urothelial Carcinoma, BRCA; Breast invasive carcinoma, CESC; Cervical squamous cell carcinoma and endocervical adenocarcinoma, CHOL; Cholangiocarcinoma, COAD; Colon adenocarcinoma, DLBCL; Lymphoid Neoplasm Diffuse Large B-cell Lymphoma, ESCA; Esophageal carcinoma, GBM; Glioblastoma multiforme, HNSC; Head and Neck squamous cell carcinoma, KICH; Kidney Chromophobe, KIRC; Kidney renal clear cell carcinoma, LAML; acute myeloid leukemia, LGG; Brain Lower Grade Glioma, LIHC; Liver hepatocellular carcinoma, LUAD; Lung adenocarcinoma, LUSC; Lung squamous cell carcinoma, MESO; Mesothelioma, OV; Ovarian serous cystadenocarcinoma, PAAD; Pancreatic adenocarcinoma, PCPG; Pheochromocytoma and Paraganglioma, PRAD; Prostate adenocarcinoma, READ; Rectum adenocarcinoma, SRC; Sarcoma, SKCM; Skin Cutaneous Melanoma, STAD; Stomach adenocarcinoma, TGCT; Testicular Germ Cell Tumors, THCA; Thyroid carcinoma, THYM; Thymoma, UCEC; Uterine Corpus Endometrial Carcinoma, UCS; Uterine Carcinosarcoma, UVM; Uveal Melanoma.

FIG. 24 (includes FIGS. 24a-24c) shows the correlations between persistent mutations and tumor aneuploidy. (a) A moderate degree of correlation was observed between pTMB and the fraction of genome with allelic imbalance in the five ICB cohorts analyzed (Spearman p range: 0.39-0.60, p<0.01), indicating that tumors with higher levels of aneuploidy tend to have higher pTMB. (b) Similar levels of correlation were observed between pTMB and the fraction of genome with multiple (>2) copies (Spearman p range: 0.36-0.63, p<0.05). (c) pTMB was weakly anti-correlated with the fraction of genome at single copy number (total CN=1) in the melanoma, mesothelioma, and NSCLC ICB cohorts (Spearman p range: −0.41, −0.14, p<0.05). Spearman's rank correlation coefficients are shown, each tumor sample represents a point and points are color coded based on tumor response on ICB.

FIG. 25 (includes FIGS. 25a-25d) shows the assessment of genomic characteristics of loci harboring persistent vs loss-prone mutations in TOGA tumors. (a) Genomic regions that are susceptible to limitations of NGS analysis, such as uncertain alignments or variant calls, were infrequent in the somatic mutation call set analyzed (MC3) and bad similar representation in the persistent and loss-prone mutation categories (n=9,242). (b) Similarly, the difference in GC composition of the immediate bases (9-mers) surrounding persistent and loss-prone mutations was negligible (n=9,242; Cohen's d=0.08, persistent mean=0.54, loss-prone mean=0.52). Persistent and loss-prone mutations were found to have similar replication timing in (c) melanoma (TCGA-SKCM, n=109, Cohen's d=−0.035) and (d) NSCLC (combined set of TCGA-LUAD and TCGA-LUSC, n=982, Cohen's d=−0.032). Stacked bar plots depict the frequency of mutations in the five quantiles of replication timing. H, MH, M, ML, and L indicate mutations in the highest to lowest quintiles of replication timing in order; as an example, mutations in earliest replicating regions are marked as H.

FIG. 26 (includes FIGS. 26a-26c) shows the context dependence of the association between pTMB and overall survival. (a) The association between persistent mutations and TMB with overall survival was assessed for 8,925 individuals across 31 cohorts in TCGA. For each tumor type and stage combination, a Cox proportional-hazards (CoxPH) model predicting the overall survival is built for each of seven features (TMB, persistent mutations-pTMB, clonal pTMB, multi-copy mutations, clonal multi-copy mutations, only-copy mutations, clonal only-copy mutations; Continuous CoxPH model). In 21 tumor types shown, an increase in at least one of the seven features listed was associated with longer overall survival and a second CoxPH model was used to assess the difference in overall survival between tumors in the top third and bottom two thirds of predicted risk (Categorical CoxPH model). Heatmap cells depict the Z-score of the model coefficient from categorical CoxPH model. Grey indicates cases where an increase in feature value is associated with shorter survival. A significant association of pTMB with prolonged overall survival was noted for lung squamous cell carcinoma, melanoma and UCS. (b) Patients with early stage (I, II, and III) squamous lung cancer (n=464) harboring a high PTMB (low risk) had a significantly longer overall survival compared to patients in the low pTMB (high risk) group especially when clonal pTMB was considered (pTMB: 56.27 vs 43.86 months, log-rank p=0.085, clonal pTMB: 60.48 vs 35.32 months, log-rank p=0.028), while no difference was found between high vs. low TMB risk groups (TMB: 55.16 vs 54.37 months, log-rank p=0.50). (c) Similarly, for patients with early stage melanoma (n=99), tumors with higher PTMB (low risk) had a significantly longer overall survival compared to those with low pTMB especially when clonal pTMB was considered (pTMB: 65.83 vs 23.69 months, log-rank p=0.036; clonal pTMB: 65.83 vs 23.69 months, log-rank p=0.013); while no such difference was observed for TMB-high vs low tumors (35.15 vs 26.97 months, log-rank p=0.98).

FIG. 27 (includes FIGS. 27a-27c) shows the evaluation of the association between pTMB computed by gene panel NGS and response to immune checkpoint blockade. TMB and pTMB estimates were derived by restricting analysis to the targeted intervals of a commonly used commercial 309 gene panel (FoundationOne-CDx) through an in silico simulation and compared between response groups. pTMB better distinguished between responding and non-responding tumors in melanoma (a; pTMB MW p=1.3e-07 vs TMB MW p=1.2e-05) and the NSCLC-Shim cohort (c; pTMB MW p=6.7e-04 vs TMB MW p=0.014). Comparison of TMB and pTMB between response groups using original estimates from whole exome sequence (WES) are included in each row for reference. pTMB and TMB had similar performance in the NSCLC-Anagnostou cohort (b); which was likely driven by the smaller size of this cohort. Boxplots depict the median value and hinges correspond to the first and third quartiles. The whiskers extend from the corresponding hinge to the furthest value within 1.5*the interquartile range from the hinge.

FIG. 28 shows the transcriptomic profiling of on-immunotherapy melanomas reveals an upregulation of inflammatory pathways in tumors harboring a high pTMB. Gene set enrichment analyses of on-treatment pTMB-high (n-9) vs. pTMB-low (n=22) melanomas revealed a marked enrichment of interferon-γ and inflammatory responses in the tumor microenvironment of PTMB-high vs pTMB-low tumors after ICB. In contrast, this profile was not observed in the tumor microenvironment of TMB-high (n=8) compared to TMB-low (n=23) melanomas. Pathways with a minimum adjusted p-value of 1e-05 in pTMB high vs low comparison are shown. The T Cell Inflamed GEP gene set was derived from Cristescu et al., Science, 2018 and the Inflammatory Response gene set was derived by Ayers et al., J Clin Invest, 2017, while the remainder of gene sets were included in the Molecular Signatures Database (Methods). Abbreviations; BC: Biocarta, HM: Hallmark, KG: KEGG, RT: Reactome, EMT: Epithelial-mesenchymal transition, Cyt. Cytokine, Rec. Receptor, Med: Mediated.

### DETAILED DESCRIPTION

Mesothelioma is a rare and fatal cancer with limited therapeutic options until the recent approval of combination immune checkpoint blockade. Disclosed herein are results of the phase 2 PrE0505 trial (NCT02899195) of the anti-PD-L1 antibody, durvalumab, plus platinum-pemetrexed chemotherapy for patients with previously untreated unresectable pleural mesothelioma. The primary endpoint was overall survival compared to historic control with cisplatin and pemetrexed chemotherapy while secondary and exploratory endpoints included safety, progression-free survival, and biomarkers of response. The combination of durvalumab with chemotherapy met the pre-specified primary endpoint reaching a median survival of 20.4 months vs. 12.1 months with historical control. Treatment-emergent adverse events were consistent with known side effects of chemotherapy and all adverse events due to immunotherapy were grade ≤2. Integrated genomic and immune cell repertoire analyses revealed that a higher immunogenic mutation burden coupled with a more diverse T cell repertoire were linked with favorable clinical outcome. Structural genome-wide analyses demonstrated a higher degree of genomic instability in responding tumors of epithelioid histology. Patients with germline alterations in cancer predisposing genes, especially those involved in DNA repair, were more likely to attain long term survival. Our findings indicate that concurrent durvalumab with platinum-based chemotherapy has promising clinical activity and that responses are driven by the complex genomic background of malignant pleural mesothelioma.

Following the non-small cell lung cancer paradigm, where the combination of first line chemotherapy with PD-1 pathway blockade has become a standard approach for advanced disease¹⁴, chemo-immunotherapy is currently being explored in MPM. In the first-line setting, the phase 2 DREAM study of durvalumab with chemotherapy achieved its primary endpoint of progression-free survival at 6 months and showed the regimen to be tolerable and active in this setting¹⁵. Furthermore, the combination of the anti-CTLA-4 antibody, ipilimumab, with the anti-PD-1 antibody, nivolumab, has been shown to improve survival for previous untreated patients when compared to chemotherapy with robust efficacy being limited to non-epithelioid histology¹⁶.

While several studies have expanded our understanding of the genomic landscape of MPM and identified putative actionable alterations, these have not been translated to therapeutic progress¹⁷⁻¹⁹. More than 50% of MPMs carry germline or somatic mutations in genes involved in DNA repair and homologous recombination^{3,20}. BAP1, a nuclear ubiquitin carboxyterminal hydrolase has been reported to be frequently mutated in the germline and tumor cells of patients with MPM17,18,20. Heterozygous germline BAP1 alterations predispose to mesothelioma especially in the context of asbestos exposure²¹ and similarly, germline BLM mutations may increase susceptibility to asbestos carcinogenesis and emergence of mesothelioma²². Inactivation of tumor suppressor genes such as BAP1, NF2, CDKN2A, TP53 and SETD2 by sequence or structural alterations is thought to be the predominant oncogenic mechanism for MPM¹⁷. Notably, MPM harbors a low tumor mutation burden (TMB) of less than 2 nonsynonymous mutations per megabase^{17,18} and has therefore been considered a tumor with low neoantigen-driven immunogenicity. Nevertheless, the promising clinical efficacy of immune checkpoint blockade for MPM calls for in-depth genomic and functional analyses to investigate the mechanisms of therapeutic response and resistance. As further disclosed below, the combination of durvalumab with platinum-based chemotherapy in a phase 2 clinical trial was investigated including to establish safety and efficacy and explore genomic and immunologic features of response in patients with unresectable MPM.

### Methods of Treatment

In certain embodiments, the methods embodied herein, identifying a mammal as having cancer.

1) obtaining a biological sample from the subject: 2) identifying germline or somatic mutations in the subject's genome; 3) identifying copy number profiles across the genome; 4) analyzing T cell receptor (TCR) clonotypes; and 5) identifying subjects responsive to chemotherapy, immunotherapy or combined chemotherapy and immunotherapy. In certain aspects, the identified subjects may be administered a chemotherapeutic, an immunotherapeutic or a chemotherapeutic and an immunotherapeutic, and, thereby treating the subject.

In certain embodiments, a subject is diagnosed as having cancer, e.g. early stage cancer. In certain embodiments, the type of cancer is identified and the cancer is treated by various therapeutics, including therapeutics specific for the type of cancer, including chemotherapy, immunotherapy or combined chemotherapy and immunotherapy.

The cancer treatment may include surgery, adjuvant chemotherapy, neoadjuvant chemotherapy, radiation therapy, hormone therapy, cytotoxic therapy, immunotherapy, adoptive T cell therapy, targeted therapy, or any combinations thereof.

The method also can include administering to the mammal a cancer treatment (e.g., surgery, adjuvant chemotherapy, neoadjuvant chemotherapy, radiation therapy, hormone therapy, cytotoxic therapy, immunotherapy, adoptive T cell therapy, targeted therapy, or any combinations thereof).

The mammal can be monitored for the presence of cancer after administration of the cancer treatment.

Cancer therapies in general also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy. The chemotherapy may also be administered at low, continuous doses which is known as metronomic chemotherapy.

Yet further combination chemotherapies include, for example, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammal1 and calicheamicin omegal1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone, anti-adrenals such as mitotane, trilostane; folic acid replenisher such as frolinic acid; aceglatone; aldophosphamide glycoside; aminolevulinic acid; eniluracil, amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine, maytansinoids such as maytansine and ansamitocins; mitoguazone; mitoxantrone; mopidanmol; nitraerine; pentostatin; phenamet, pirarubicin; losoxantrone; podophyllinic acid; 2-ethylhydrazide; procarbazine; PSK polysaccharide complex, razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; carboplatin, procarbazine, plicomycin, gemcitabien, navelbine, farnesyl-protein transferase inhibitors, transplatinum; and pharmaceutically acceptable salts, acids or derivatives of any of the above.

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells as well as genetically engineered variants of these cell types modified to express chimeric antigen receptors.

The immunotherapy may comprise suppression of T regulatory cells (Tregs), myeloid derived suppressor cells (MDSCs) and cancer associated fibroblasts (CAFs). In some embodiments, the immunotherapy is a tumor vaccine (e.g., whole tumor cell vaccines, peptides, and recombinant tumor associated antigen vaccines), or adoptive cellular therapies (ACT) (e.g., T cells, natural killer cells, TILs, and LAK cells). The T cells may be engineered with chimeric antigen receptors (CARs) or T cell receptors (TCRs) to specific tumor antigens. As used herein, a chimeric antigen receptor (or CAR) may refer to any engineered receptor specific for an antigen of interest that, when expressed in a T cell, confers the specificity of the CAR onto the T cell. Once created using standard molecular techniques, a T cell expressing a chimeric antigen receptor may be introduced into a patient, as with a technique such as adoptive cell transfer. In some aspects, the T cells are activated CD4 and/or CD8 T cells in the individual which are characterized by γ-IFN-producing CD4 and/or CD8 T cells and/or enhanced cytolytic activity relative to prior to the administration of the combination. The CD4 and/or CD8 T cells may exhibit increased release of cytokines selected from the group consisting of IFN-γ, TNF-α and interleukins. The CD4 and/or CD8 T cells can be effector memory T cells. In certain embodiments, the CD4 and/or CD8 effector memory T cells are characterized by having the expression of CD44^{high} CD62L^{low}.

The immunotherapy may be a cancer vaccine comprising one or more cancer antigens, in particular a protein or an immunogenic fragment thereof, DNA or RNA encoding said cancer antigen, in particular a protein or an immunogenic fragment thereof, cancer cell lysates, and/or protein preparations from tumor cells. As used herein, a cancer antigen is an antigenic substance present in cancer cells. In principle, any protein produced in a cancer cell that has an abnormal structure due to mutation can act as a cancer antigen. In principle, cancer antigens can be products of mutated Oncogenes and tumor suppressor genes, products of other mutated genes, overexpressed or aberrantly expressed cellular proteins, cancer antigens produced by oncogenic viruses, oncofetal antigens, altered cell surface glycolipids and glycoproteins, or cell type-specific differentiation antigens. Examples of cancer antigens include the abnormal products of ras and p53 genes. Other examples include tissue differentiation antigens, mutant protein antigens, oncogenic viral antigens, cancer-testis antigens and vascular or stromal specific antigens Tissue differentiation antigens are those that are specific to a certain type of tissue. Mutant protein antigens are likely to be much more specific to cancer cells because normal cells shouldn't contain these proteins. Normal cells will display the normal protein antigen on their MHC molecules, whereas cancer cells will display the mutant version Some viral proteins are implicated in forming cancer, and some viral antigens are also cancer antigens Cancer-testis antigens are antigens expressed primarily in the germ cells of the testes, but also in fetal ovaries and the trophoblast. Some cancer cells aberrantly express these proteins and therefore present these antigens, allowing attack by T-cells specific to these antigens. Exemplary antigens of this type are CTAG1 B and MAGEA1 as well as Rindopepimut, a 14-mer intradermal injectable peptide vaccine targeted against epidermal growth factor receptor (EGFR) vll1 variant. Rindopepimut is particularly suitable for treating glioblastoma when used in combination with an inhibitor of the CD95/CD95L signaling system as described herein. Also, proteins that are normally produced in very low quantities, but whose production is dramatically increased in cancer cells, may trigger an immune response. An example of such a protein is the enzyme tyrosinase, which is required for melanin production. Normally tyrosinase is produced in minute quantities but its levels are very much elevated in melanoma cells. Oncofetal antigens are another important class of cancer antigens. Examples are alphafetoprotein (AFP) and carcinoembryonic antigen (CEA). These proteins are normally produced in the early stages of embryonic development and disappear by the time the immune system is fully developed. Thus self-tolerance does not develop against these antigens. Abnormal proteins are also produced by cells infected with oncoviruses, e.g. EBV and HPV. Cells infected by these viruses contain latent viral DNA which is transcribed and the resulting protein produces an immune response. A cancer vaccine may include a peptide cancer vaccine, which in some embodiments is a personalized peptide vaccine. In some embodiments, the peptide cancer vaccine is a multivalent long peptide vaccine, a multi-peptide vaccine, a peptide cocktail vaccine, a hybrid peptide vaccine, or a peptide-pulsed dendritic cell vaccine

The immunotherapy may be an antibody, such as part of a polyclonal antibody preparation, or may be a monoclonal antibody. The antibody may be a humanized antibody, a chimeric antibody, an antibody fragment, a bispecific antibody or a single chain antibody. An antibody as disclosed herein includes an antibody fragment, such as, but not limited to, Fab, Fab′ and F (ab′) 2, Fd, single-chain Fvs (scFv), single-chain antibodies, disulfide-linked Fvs (sdfv) and fragments including either a VL or VH domain. In some aspects, the antibody or fragment thereof specifically binds epidermal growth factor receptor (EGFR1, Erb-B1), HER2/neu (Erb-B2), CD20, Vascular endothelial growth factor (VEGF), insulin-like growth factor receptor (IGF-1R), TRAIL-receptor, epithelial cell adhesion molecule, carcino-embryonic antigen, Prostate-specific membrane antigen, Mucin-1, CD30, CD33, or CD40.

Examples of monoclonal antibodies include, without limitation, trastuzumab (anti-HER2/neu antibody); Pertuzumab (anti-HER2 mAb); cetuximab (chimeric monoclonal antibody to epidermal growth factor receptor EGFR); panitumumab (anti-EGFR antibody); nimotuzumab (anti-EGFR antibody); Zalutumumab (anti-EGFR mAb); Necitumumab (anti-EGFR mAb); MDX-210 (humanized anti-HER-2 bispecific antibody); MDX-210 (humanized anti-HER-2 bispecific antibody); MDX-447 (humanized anti-EGF receptor bispecific antibody); Rituximab (chimeric murine/human anti-CD20 mAb); Obinutuzumab (anti-CD20 mAb); Ofatumumab (anti-CD20 mAb); Tositumumab-1131 (anti-CD20 mAb); Ibritumomab tiuxetan (anti-CD20 mAb); Bevacizumab (anti-VEGF mAb); Ramucirumab (anti-VEGFR2 mAb); Ranibizumab (anti-VEGF mAb); Aflibercept (extracellular domains of VEGFR1 and VEGFR2 fused to IgG1 Fc); AMG386 (angiopoietin-1 and -2 binding peptide fused to IgG1 Fc); Dalotuzumab (anti-IGF-1R mAb); Gemtuzumab ozogamicin (anti-CD33 mAb); Alemtuzumab (anti-Campath-1/CD52 mAb); Brentuximab vedotin (anti-CD30 mAb); Catumaxomab (bispecific mAb that targets epithelial cell adhesion molecule and CD3); Naptumomab (anti-ST4 mAb); Girentuximab (anti-Carbonic anhydrase ix); or Farletuzumab (anti-folate receptor). Other examples include antibodies such as Panorex™ (17-1A) (murine monoclonal antibody); Panorex (MAb17-1A) (chimeric murine monoclonal antibody); BEC2 (ami-idiotypic mAb, mimics the GD epitope) (with BCG); Oncolym (Lym-1 monoclonal antibody); SMART M195 Ab, humanized 13′ 1 LYM-1 (Oncolym), Ovarex (B43.13, anti-idiotypic mouse mAb); 3622W94 mAb that binds to EGP40 (17-1A) pancarcinoma antigen on adenocarcinomas; Zenapax (SMART Anti-Tac (IL-2 receptor); SMART M195 Ab, humanized Ab, humanized); NovoMAb-G2 (pancarcinoma specific Ab); TNT (chimeric mAb to histone antigens); TNT (chimeric mAb to histone antigens); Gliomab-H (Monoclonals-Humanized Abs); GNI-250 Mab; EMD-72000 (chimeric-EGF antagonist); LymphoCide (humanized IL.L.2 antibody); and MDX-260 bispecific, targets GD-2, ANA Ab, SMART IDIO Ab, SMART ABL 364 Ab or ImmuRAIT-CEA. Further examples of antibodies include Zanulimumab (anti-CD4 mAb), Keliximab (anti-CD4 mAb); Ipilimumab (MDX-101; anti-CTLA-4 mAb); Tremilimumab (anti-CTLA-4 mAb); (Daclizumab (anti-CD25/IL-2R mAb); Basiliximab (anti-CD25/IL-2R mAb); MDX-1106 (anti-PD1 mAb); antibody to GITR, GC1008 (anti-TGF-β antibody); metelimumab/CAT-192 (anti-TGF-β antibody); lerdelimumab/CAT-152 (anti-TGF-B antibody); ID11 (anti-TGF-β antibody); Denosumab (anti-RANKL mAb); BMS-663513 (humanized anti-4-1BB mAb); SGN-40 (humanized anti-CD40 mAb); CP870,893 (human anti-CD40 mAb); Infliximab (chimeric anti-TNF mAb, Adalimumab (human anti-TNF mAb); Certolizumab (humanized Fab anti-TNF); Golimumab (anti-TNF); Etanercept (Extracellular domain of TNFR fused to IgG1 Fc); Belatacept (Extracellular domain of CTLA-4 fused to Fc); Abatacept (Extracellular domain of CTLA-4 fused to Fc); Belimumab (anti-B Lymphocyte stimulator); Muromonab-CD3 (anti-CD3 mAb); Otelixizumab (anti-CD3 mAb); Teplizumab (anti-CD3 mAb); Tocilizumab (anti-IL6R mAb); REGN88 (anti-IL6R mAb); Ustekinumab (anti-IL-12/23 mAb); Briakinumab (anti-IL-12/23 mAb); Natalizumab (anti-α4 integrin); Vedolizumab (anti-α4 β7 integrin mAb); Tl h (anti-CD6 mAb); Epratuzumab (anti-CD22 mAb); Efalizumab (anti-CD11a mAb); and Atacicept (extracellular domain of transmembrane activator and calcium-modulating ligand interactor fused with Fc).

As discussed above, methods are provided for assessing, monitoring, and/or treating mammals (e.g., humans) having, or suspected of having, cancer. For example, a sample (e.g., a blood sample) obtained from a mammal can be assessed to determine:
1) whether the mammal (e.g. human) subject is identified as having a high number of mutations in haploid regions of the genome (which is predictive of responsiveness to chemotherapeutic and/or immunotherapeutic treatment); and/or
2) whether the mammal (e.g. human) subject is identified as having a high number of mutations in polyploid regions of the genome (which is predictive of responsiveness to chemotherapeutic and/or immunotherapeutic treatment); and/or
3) whether the mammal (e.g. human) subject is identified as having a high number of mutations in haploid and polyploid regions of the genome (which is predictive of responsiveness to chemotherapeutic and/or immunotherapeutic treatment).

Following such identifying, one or more cancer treatments can be administered to the mammal to treat the mammal.

In some cases, during or after the course of a cancer treatment (e.g., any of the cancer treatments described herein), a mammal can undergo monitoring (or be selected for increased monitoring) and/or further diagnostic testing

Any appropriate mammal can be assessed, monitored, and/or treated as described herein. A mammal can be a mammal having cancer. A mammal can be a mammal suspected of having cancer. Examples of mammals that can be assessed, monitored, and/or treated as described herein include, without limitation, humans, primates such as monkeys, dogs, cats, horses, cows, pigs, sheep, mice, and rats.

Any appropriate sample from a mammal can be assessed as described herein. In some cases, a sample can be fluid sample (e.g., a liquid biopsy). Examples of samples include, without limitation, blood (e.g., whole blood, serum, or plasma), amnion, tissue, urine, cerebrospinal fluid, saliva, sputum, broncho-alveolar lavage, bile, lymphatic fluid, cyst fluid, stool, ascites, pap smears, breast milk, and exhaled breath condensate.

As discussed above, when treating a mammal having, or suspected of having, cancer as described herein, the mammal can be administered one or more cancer treatments. A cancer treatment can be any appropriate cancer treatment. One or more cancer treatments described herein can be administered to a mammal at any appropriate frequency (e.g., once or multiple times over a period of time ranging from days to weeks). Examples of cancer treatments include, without limitation adjuvant chemotherapy, neoadjuvant chemotherapy, radiation therapy, hormone therapy, cytotoxic therapy, immunotherapy, adoptive T cell therapy (e.g., chimeric antigen receptors and/or T cells having wild-type or modified T cell receptors), targeted therapy such as administration of kinase inhibitors (e.g., kinase inhibitors that target a particular genetic lesion, such as a translocation or mutation), (e.g. a kinase inhibitor, an antibody, a bispecific antibody), signal transduction inhibitors, bispecific antibodies or antibody fragments (e.g., BiTEs), monoclonal antibodies, immune checkpoint inhibitors, surgery (e.g., surgical resection), or any combination of the above. In some cases, a cancer treatment can reduce the severity of the cancer, reduce a symptom of the cancer, and/or to reduce the number of cancer cells present within the mammal.

In some cases, a cancer treatment can include an immune checkpoint inhibitor. Non-limiting examples of immune checkpoint inhibitors include nivolumab (Opdivo), pembrolizumab (Keytruda), atezolizumab (tecentriq), avelumab (bavencio), dorvalumab (imfinzi), ipilimumab (yervoy). See, e.g., Pardoll (2012) Nat. Rev Cancer 12: 252-264; San et al. (2017) Eor Rev Med Pharmacol Sci 21(6): 1198-1205; Hamanisbi et al. (2015) J. Clin. Oncol 33(34): 4015-22; Brahmer et al. (2012) N Engl J Med 366(26): 2455-65; Ricciuti et al. (2017) J. Thorac Oncol. 12(5) e51-e55; Ellis et al. (2017) Clio Lung Cancer pii: S1525-7304(17)30043-8; Zou and Awad (2017) Ann Oncol 28(4): 685-687; Sorscher (2017) N Engl J Med 376(10: 996-7; Hui et al. (2017) Ann Oncol 28(4): 874-881, Vansteenkiste et al. (2017) Expert Opin Biol Ther 17(6): 781-789; Hellmann et al. (2017) Lancet Oncol. 18(1): 31-41; Chen (2017) J. Chin Med Assoc 80(1): 7-14.

In some cases, a cancer treatment can be an adoptive T cell therapy (e.g., chimeric antigen receptors and/or T cells having wild-type or modified T cell receptors). See, e.g., Rosenberg and Restifo (2015) Science 348(6230): 62-68; Chang and Chen (2017) Trends Mol Med 23(5): 430-450; Yee and Lizee (2016) Cancer J. 23(2): 144-148, Chen et al. (2016) Oncoimmunology 6(2): e1273302, US 2016/0194404; US 2014/0050788; US 2014/0271635; U.S. Pat. No. 9,233,125; incorporated by reference in their entirety herein.

As discussed above, in some cases, a cancer treatment can be a chemotherapeutic agent

### Study Design

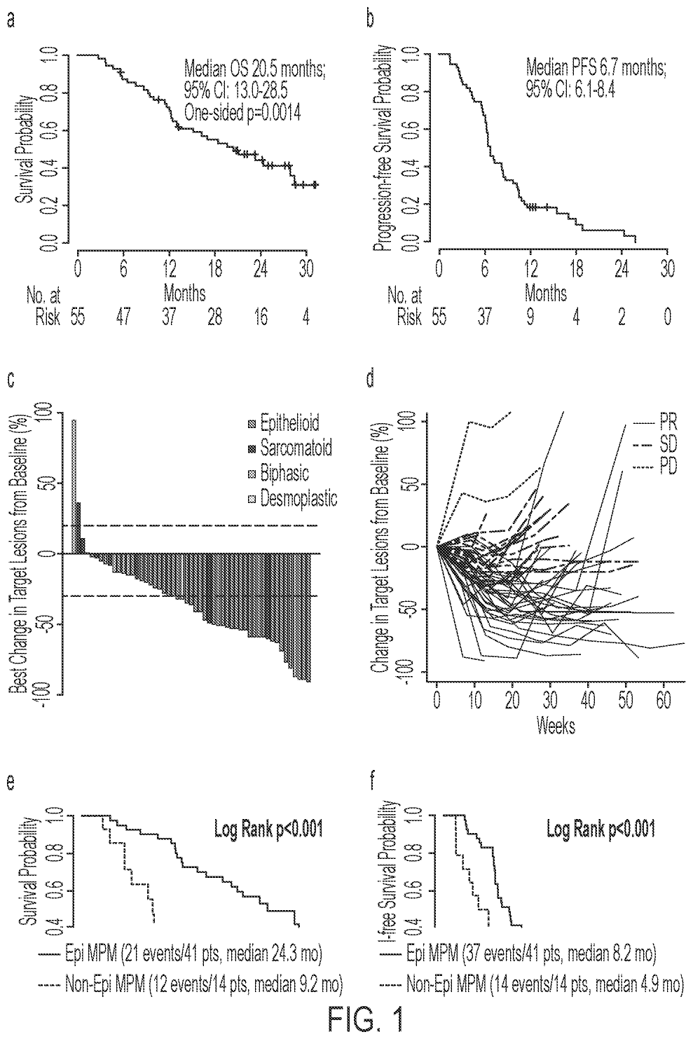

PrE0505, a phase 2 single arm multicenter study, enrolled human patients with previously untreated unresectable MPM (NCT02899195, FIG. 1). Patients received durvalumab (at a fixed dose of 1120 mg IV) given once every 3 weeks in combination with pemetrexed and cisplatin at their standard doses for up to 6 cycles. Substitution of carboplatin for cisplatin was permitted on cycle 1 for patients with a glomerular filtration rate (GFR) of ≥45 mL/min but ≤60 mL/min, another documented contraindication to cisplatin (e.g. hearing loss), or due to cisplatin toxicity during treatment. Patients with stable or responding tumors after concurrent therapy, continued on maintenance durvalumab for a maximum duration of one year from the first study treatment. A protocol-defined safety review was performed after enrollment of the first 6 and 15 patients. Pre-specified dose-limiting toxicities during the safety run-in period included any immune-mediated adverse event of ≥grade 4 or any non-resolving immune-mediated adverse event of ≥grade 3 during the first two cycles of therapy.

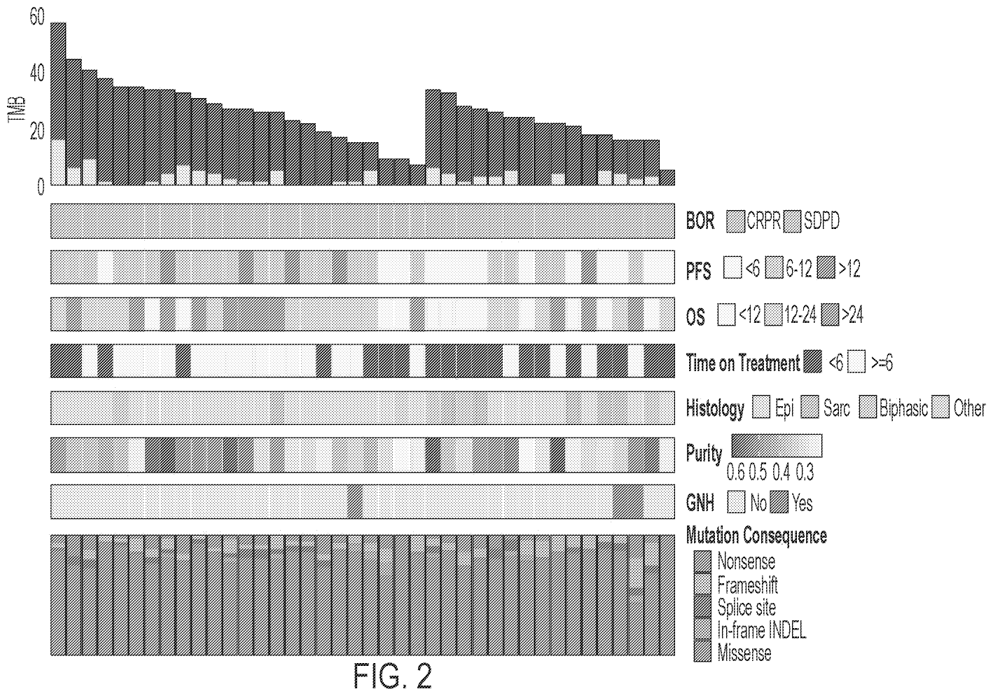

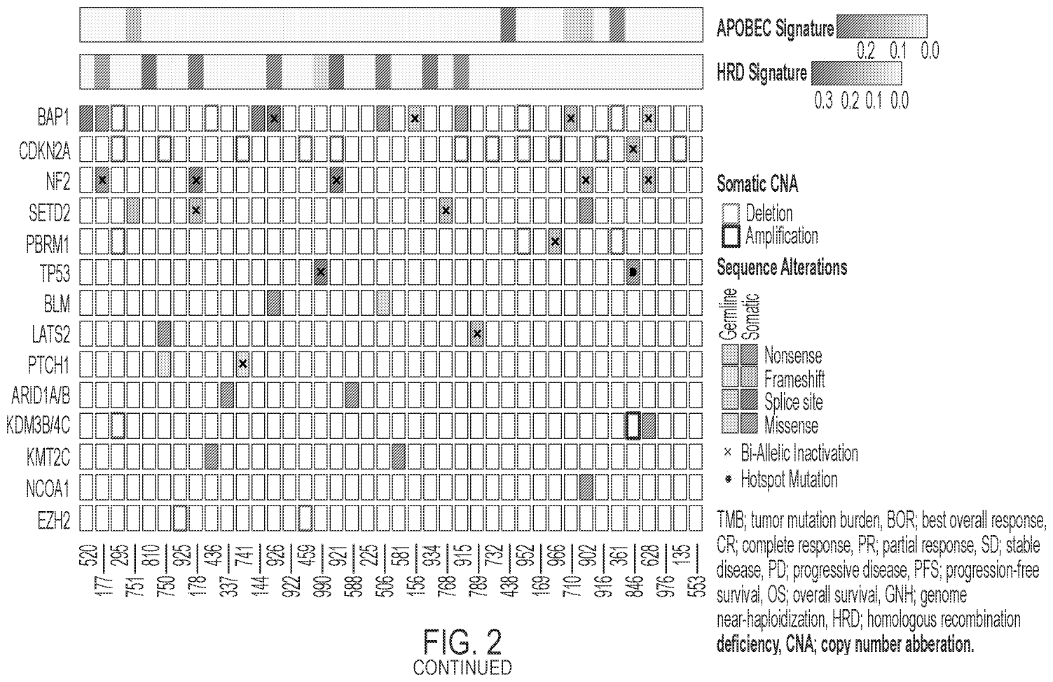

The primary endpoint of the study was overall survival (OS), defined as time from study registration to death from any cause. Patients last known to be alive were censored at their date of last follow-up. We assumed a null hypothesis that the median OS with chemo-immunotherapy would be equal to the OS of 12 months with pemetrexed/cisplatin alone (historical control). The total planned enrollment of 55 patients (50 eligible) with 32 events, allowed for 90% power to detect a 37% reduction in the OS hazard rate of 0.058 to 0.037 based on Wald test for the log failure rate parameter using one-sided type I error rate of 10%. This would correspond to a 58% improvement in the median OS from 12 to 19 months⁴. Secondary endpoints included progression-free survival (PFS), best objective response, and toxicity (Methods). PFS was defined as the time from study registration to documented disease progression or death from any cause, whichever occurred first. Patients who did not experience an event of interest were censored at the date they were last known to be alive and progression-free. Exploratory endpoints included investigating the genomic and immunologic underpinning of response to chemo-immunotherapy; to this end we performed whole exome sequencing (WES), coupled with genome-wide focal and large-scale copy number aberration analysis and sequence deconvolution (Methods). In parallel, we evaluated the intra-tumoral T cell repertoire and functional neoantigen-specific T cell responses (Methods below and FIGS. 1 and 2).

### Participants

PrE0505 enrolled 55 patients at 15 academic and community cancer centers in the United States. Eligible patients were 18 years of age or older and had histologically unselected MPM that was deemed to be surgically unresectable, an Eastern Cooperative Oncology Group performance-status score of 0 or 1, adequate organ function including GFR of ≥45 mL/min, and measurable disease by RECIST 1.1 modified for pleural mesothelioma^{23,24}. Key exclusion criteria were immunodeficiency, ongoing systemic immunosuppressive therapy, active autoimmune or infectious disease, and clinically significant concurrent cancer. Demographics and disease characteristics are summarized in Table 1; median age was 68 (range 35-83), the majority of patients were male (82%) and 75% of tumors were of epithelioid histology. Patients who had continued clinical benefit by investigator assessment (n=20) were allowed to continue on treatment past radiographic progression.

### Efficacy Analyses

All patients were included in the eligible population for efficacy analyses. The median follow-up was 24.2 months at the time of this analysis, with 33 death events. The median OS for all patients enrolled was 20.4 months (95% CI: 13.0 to 28.5, 80% CI: 15.1, 27.9) and was significantly longer than the historic control of 12 months (one-sided p=0.0014; FIG. 1a), with an observed hazard rate of 0.034. The estimated percentages of patients alive at 6, 12 and 24 months were 87.2%, 70.4% and 44.2% respectively. Median progression-free survival was 6.7 months (95% CI: 6.1 to 8.4, 80% CI: 6.3 to 8.2; FIG. 1b). The estimated percentages of patients alive and progression-free at 6, 12 and 24 months were 67.3%, 18.2% and 6.1% respectively. The objective response rate (ORR) was 56.4% (95% CI: 42.3% to 69.7%, and 80% CI 46.8% to 65.5%). There were no patients with a complete response (CR), 31 patients showed partial response (PR) and 20 patients had stable disease (SD). One patient was unevaluable for response due to missing follow-up disease assessments and three patients had progressive disease (PD) as best response (FIGS. 1c and 1d). In a non-predefined subgroup analysis, a significant difference in ORR, PFS and OS was noted by histology. Patients with epithelioid tumors had a higher ORR than non-epithelioid (65.9% vs. 28.6%, p=0.03). Similarly, patients with epithelioid MPM had significantly longer median OS (24.3 months vs. 9.2 months, hazard ratio-HR: 0.27, 95% CI: 0.13-0.57, p<0.001; FIG. 1e) and PFS (8.2 months vs. 4.9 months, HR: 0.30, 95% CI: 0.16-0.58, p<0.001; FIG. 1f) when compared to the non-epithelioid MPM group.

### Safety

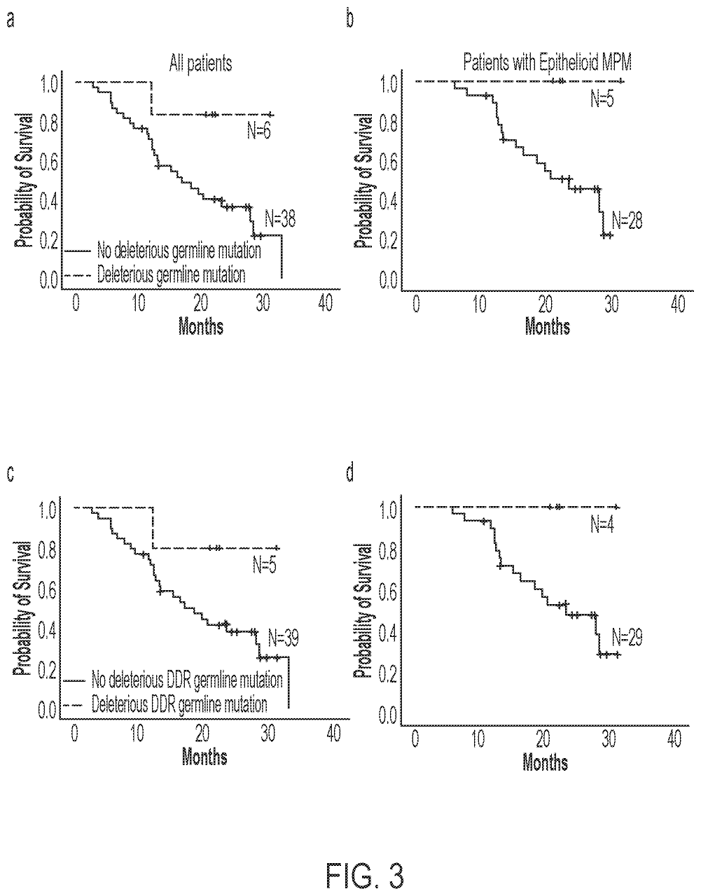

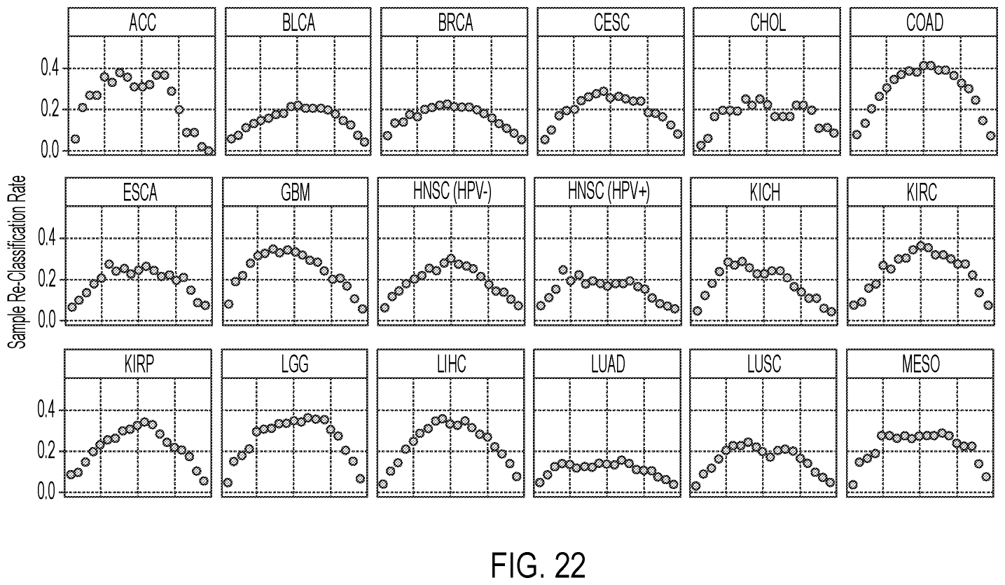

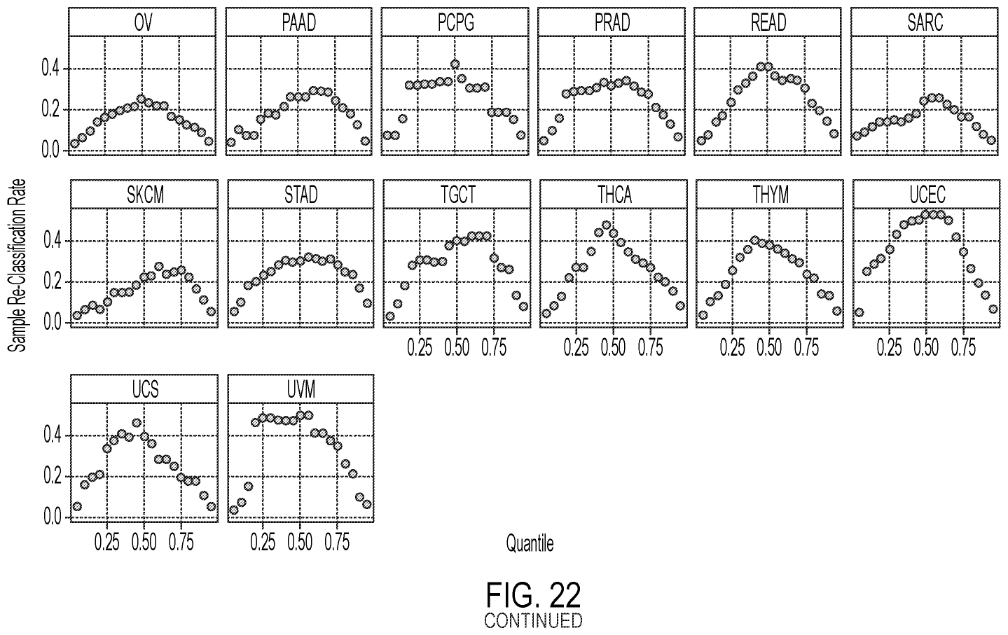

The study enrolled the full planned cohort of 55 patients after initial safety analysis; the most commonly reported treatment-emergent adverse events (TEAEs) were mostly of low grade and included fatigue (67%), nausea (56%) and anemia (56%; FIG. 3). Grade 3 or greater TEAEs occurred in 65.5% of patients and included anemia (20%), hyponatremia (9%), fatigue (7%), leucopenia (5%), thrombocytopenia (5%) and hypertension (5%); all other grade 3 or greater TEAEs occurred in <5% of patients. There were no grade 5 TEAEs. There were also no unexpected adverse events of special interest (defined as adverse events with a potential immune-mediated mechanism) and those that did occur were of grade 2 or less (FIG. 22). In terms of treatment received, 29 (53%) patients received cisplatin-based chemotherapy from the start of treatment while the rest began with carboplatin, a further seven patients among the cisplatin group switched to carboplatin during concurrent treatment. There was no statistically significant difference in progression-free or overall survival based on which platinum agent was received (FIG. 22). All patients who enrolled in the study received at least one cycle of durvalumab with chemotherapy, 87.3% (48/55) completed six cycles of concurrent treatment. Ten (18.2%) patients with epithelioid MPM completed the one-year study treatment and five (9.1%) patients discontinued the study treatment due to toxicity. The median dose intensity (of cycles received) was 100% for cisplatin (range 75-100), 100% for pemetrexed (range 75-102) and 100% for durvalumab (range 100-100). Two (7%) of 29 patients who received cisplatin received a dose reduction of cisplatin due to toxicity and four (12%) of 33 who received carboplatin received a dose reduction of carboplatin due to toxicity. One (2%) patient received a dose reduction of pemetrexed due to toxicity, while no patients received a dose reduction of durvalumab due to toxicity (FIG. 1).

### Genomic and Immunologic Exploratory Analyses

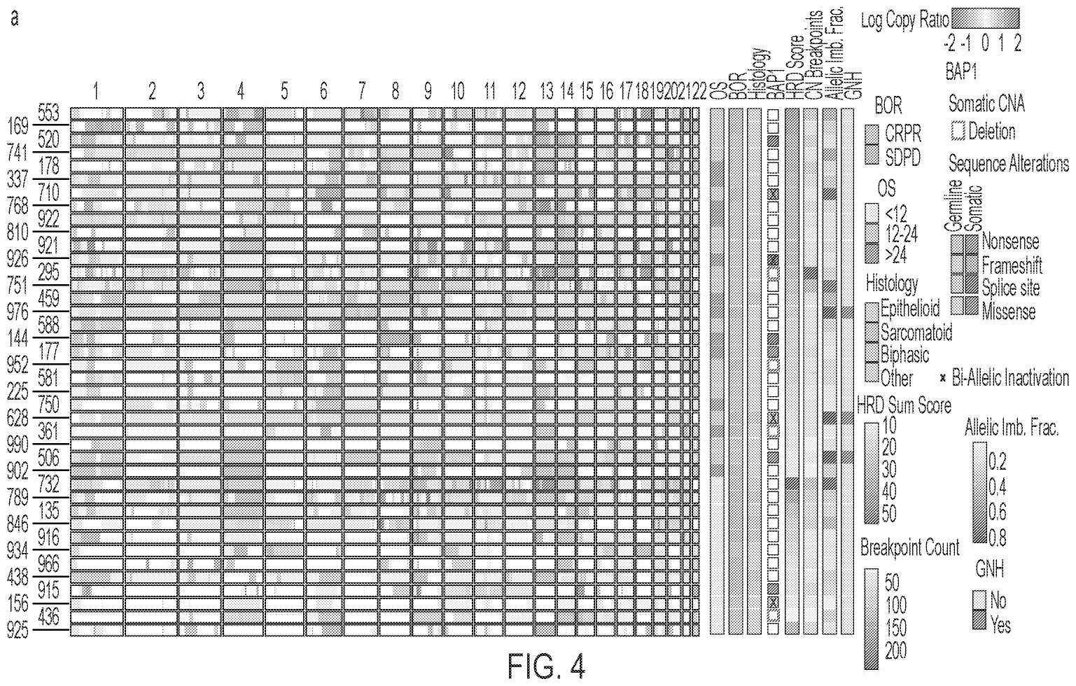

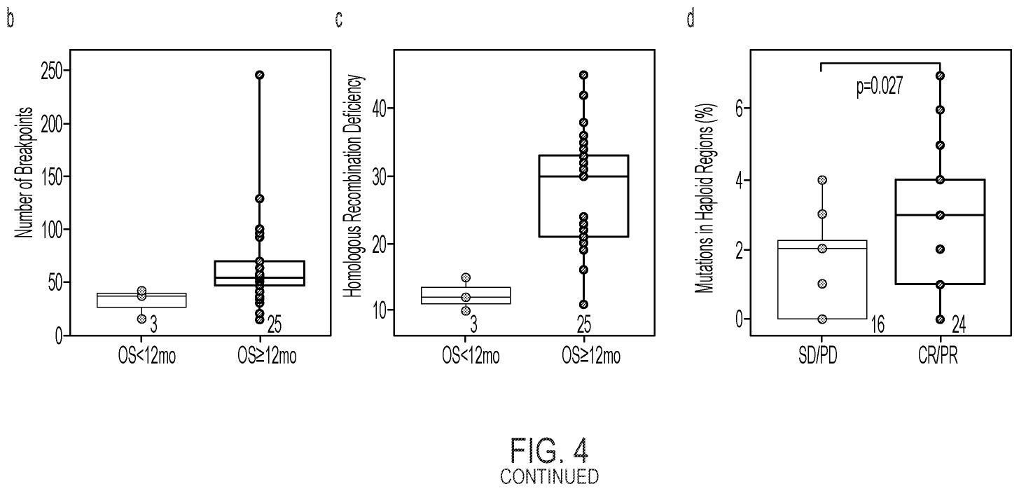

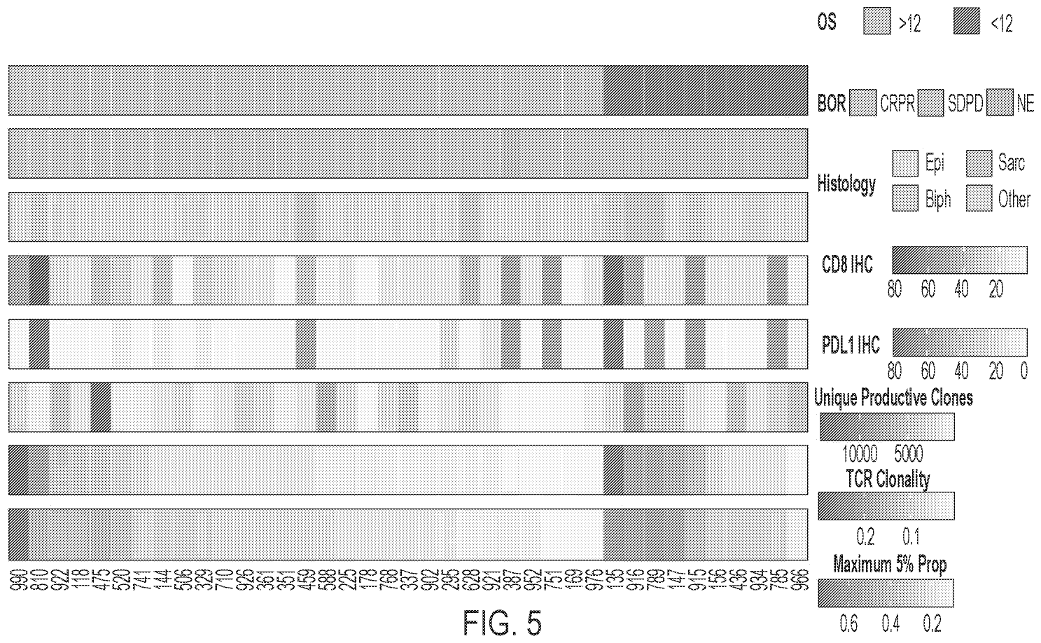

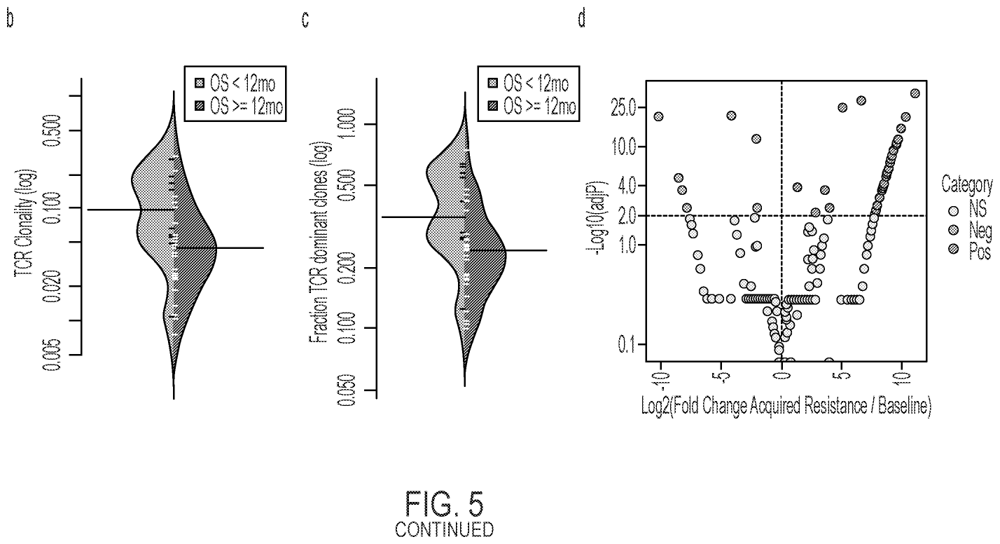

As previously shown¹⁷, MPMs in this cohort harbored a low tumor mutation burden, with some tumors harboring a higher than expected TMB in the setting of mutations in DNA damage repair genes (FIG. 4). Despite the notion that TMB may not predict response to immunotherapy for MPM, given the low mutation burden, we found that tumors from patients with a radiographic response to chemo-immunotherapy had a higher nonsynonymous missense mutation burden and a more clonal mutation repertoire compared to non-responding tumors (p=0.086 and p=0.072 respectively, FIG. 5), especially in the epithelioid group (p=0.051 and p=0.025 respectively, FIG. 5). Consistent with these findings, an APOBEC mutational signature, reflective of subclonal mutagenesis, was found to be enriched in epithelioid tumors of non-responders (p=0.031 respectively, FIG. 2). To place these findings in context with respect to the potential role of TMB in predicting response to standard-of-care treatment versus chemo-immunotherapy, we analyzed tumor whole exome sequencing data from an independent cohort of 82 MPM tumors from TCGA, which predated the era of immune checkpoint blockade. In the TCGA MPM cohort, TMB-low tumors (TMB less than or equal to the second tertile in the cohort; Methods) showed a non-significant trend towards a longer progression-free survival, suggesting that the relationship between high TMB and clinical response seen in the PrE0505 cohort may be driven by durvalumab (Methods below). In the PrE0505 cohort, a trend towards an enrichment in mutations in chromatin regulating genes was observed for patients achieving an overall survival ≥12 months (p=0.063).

We then evaluated nonsynonymous sequence alterations associated with putatively immunogenic neoantigens (immunogenic mutations-IMMs, Methods) and found an enrichment of high MHC class I IMM burden (p=0.064) as well as a higher MHC class II IMM burden (p=0.023) in responsive tumors (FIG. 5), especially for epithelioid MPM (p=0.035 and p=0.038 respectively). Interestingly, consistent with the HLA class I allele divergence hypothesis that points towards a more efficient tumor immune surveillance in the presence of HLA class I functional diversity²⁵, maximal germline physiochemical sequence divergence at the HLA-B locus was associated with radiographic response, especially for epithelioid MPM (p=0.06 and p=0.003 respectively). We subsequently tested autologous T cells for reactivity against IMM-derived neopeptides for two patients achieving long-term clinical response (Methods). TCR clonotypic expansions for neoantigens derived from the SRPK2 p.C234Y and NDUFS2 p. V412L mutations were prominent for patient 459, with sarcomatoid MPM, who had an overall survival of 33 months. Similarly, neoantigen-specific TCR expansions were noted for the IMMs SCRNI p.V334A, PSD2 p.C307Y, ZNF469 p.P3471S and CD72 p.T71A for patient 295 with epithelioid MPM and an overall survival of 21.85 months (patient remained event-free at the time of data lock).

Mesothelioma may arise in the context of germline mutations in cancer susceptibility genes, including BAP1, MLH1, MLH3, BRCA1/2 and BLM^{20,22}, however the potential impact of germline MPM-predisposing mutations on response to chemo-immunotherapy has not been previously evaluated. Patients with pathogenic germline loss-of-function mutations in MPM susceptibility genes (Methods), predominantly those involved in DNA damage repair, bad a significantly prolonged survival (log rank p-0.05 and p=0.032 for all patients and epithelioid MPM respectively, FIG. 3a-d). Interestingly, patients with sporadic BAP/mutant MPM harboring heterozygous somatic inactivating mutations did not have a better radiographic response or longer overall survival, potentially suggesting that immune surveillance mechanisms differ in the context of germline BAP1 deficiency^{26,27}. As previously described^{4,28}, patients with inactivating BAP1 germline mutations were younger (p=0.009)^{29,30} and tumors with BAP1 biallellic inactivation were found to have an enriched DNA mismatch repair deficiency-related mutational signature (p=0.003). Tumors harboring somatic BAP1 inactivating mutations were noted to have a higher clonal TMB as well as a lower fraction of genome with loss-of-heterozygosity (p=0.033 and p=0.018 respectively); notably a trend towards a lower degree of LOH was also observed for BAP1-mutant mesotheliomas in the TCGA cohort. Interestingly, these findings were not corroborated in cases harboring germline inactivating BAP1 mutations, highlighting the differences between germline and somatic genetic backgrounds. Furthermore, we found that epithelioid MPMs harboring somatic BAP1 inactivating mutations had a higher intratumoral CD8+ T cell infiltration. Analyses of transcriptomic sequence data for 6 patients with available tissue (4 BAP1 wild type and 2 BAP/mutant) revealed a higher expression level for GranzymeB in BAP1 mutant tumors, suggesting an active cytotoxic immune response in the microenvironment of BAP1 mutant tumors.

Genomic instability and particularly large scale copy number losses, are hallmarks of MPM18,29 and our large-scale copy number analyses revealed recurrent chromosomal arm losses or loss of heterozygosity (LOH) of 4p, 4q, 69, 9p, 10q, 13q, 14q, 18q and 22q as well as LOH/deletion of 3p21.1, where BAP1 lies (FIG. 4a). Regions of recurrent copy loss contained the LATS1 (69), CDKN2A (9p), LATS2 (13q) and NF2 (22q) loci. We found an enrichment for LOH of chromosomal arm 1p and hemizygous loss of chromosomal arm 6q in tumors that did not achieve a radiographic response (2 out of 24 in the responder vs 6 out of 16 in the non-responder group, p=0.04, FIG. 4a). Interestingly, loss of chromosomal arm 6q, which contains the mesothelioma driver genes LATS1, REV3L and SHPRH, was more prominent in epithelioid tumors without treatment response (2 out of 21 in the responder vs 4 out of 8 in the non-responder group, p=0.033). As DNA breaks occur frequently in mesothelioma^{17,19}, we quantified chromosomal instability by estimating the number of copy number breakpoints across the genome (Methods) and found a higher content of genome-wide breakpoints in epithelioid MPM of patients with an OS ≥12 months (p=0.053, FIG. 4b). Analysis of breakpoints of the TCGA mesothelioma cohort failed to identify such an association with overall survival (univariate Cox proportional hazards regression analysis, HR=1.36, p=0.23). Furthermore, we computed a composite homologous recombination deficiency (HRD) score, incorporating telomeric allelic imbalance, loss of heterozygosity and large-scale state transitions and found that a higher HRD score defined epithelioid tumors from patients with long-term survival (p=0.014 respectively, FIG. 4c); in contrast there was no association between a higher HRD score and outcome in the TCGA mesothelioma cohort (either as a continuous, HR=1.02, p=0.14 or binarized variable, HR=1.58, p=0.18 utilizing univariate Cox proportional hazards regression analysis). Consistent with these findings, a mutational signature of DNA double-strand break repair deficiency was more enriched in tumors of patients achieving a radiographic complete or partial response (p=0.043, FIG. 2). Three cases showed genomic near haploidization, which is a phenomenon where cells lose one copy of nearly all chromosomes followed by duplication of the remaining chromosomes, and interestingly all of these patients had an OS ≥12 months (FIG. 4a). It is plausible that mutations and associated neoantigens contained in regions of the genome with in a single copy per cancer cell cannot be eliminated under the selective pressure of therapy and therefore mediate sustained neoantigen-driven immune responses and long-term clinical benefit. Consistent with this notion, we discovered a higher number of sequence alterations contained in single copy regions of the genome in tumors from responders compared to non-responders (p=0.027, FIG. 4d); notably the overwhelming majority of these were clonal (71 out of 93, 76%). To further support these findings, we investigated the background rate of loss in regions of the genome with a single copy per cell (haploid) versus euploid regions (2 copies per cell) and analyzed somatic copy number profiles of 1,086 mesothelioma and non-small cell lung cancer tumors from TCGA (Methods). These analyses revealed that the rate of loss in haploid regions was consistently lower than that of euploid regions, supporting the notion that mutations contained in these regions are hard to eliminate and may drive a sustained anti-tumor immune response.

As reported previously¹⁵, we did not observe any association between radiographic responses, PFS or OS and PD-L1 expression on tumor cells. In looking at the tumor microenvironment of MPM, the composition of the pre-existing intra-tumoral TCR repertoire tied into the genomic footprint of MPM has not been previously investigated in the context of chemo-immunotherapy. Baseline tumors of patients with an OS ≥12 months harbored a more diverse TCR repertoire in contrast to tumors of patients with shorter overall survival, which showed a higher TCR repertoire clonality and a higher proportion of high frequency TCR clones (p=0.018 and p=0.006 respectively, FIG. Sa-c. We investigated the reshaping of the intra-tumoral T cell repertoire for three patients (295, 459 and 926) who had long term therapeutic responses but eventually developed acquired resistance, by serially sampling tumors prior to therapy and at the time of acquired resistance. Interestingly, at the time of acquired resistance, significant reshaping signified by TCR clonotypic expansions and regressions was noted such that the tumor infiltrating lymphocyte (TIL) repertoire from all 3 cases was significantly more clonal (FIG. 5d). These findings potentially suggest that an effective anti-tumor immune response is mediated by a polyclonal T-cell repertoire and dependency on fewer TCR effector cells may not be sufficient to mount an effective anti-tumor immune response.

### Discussion

The PrE0505 trial demonstrated promising rates of response, progression-free and overall survival for patients who received durvalumab with standard chemotherapy as first-line therapy for unresectable MPM. Treatment was well tolerated and there were low rates of immune-mediated toxicity. The median overall survival of 20.4 months in the PrE0505 trial is encouraging in the context of several recent phase 2 and 3 clinical trials that enrolled a similarly representative population of treatment-naïve patients^{7-9,16}. Allied to recent results from the DREAM study, these data launched the ongoing phase 3 PrE0506/DREAM3R trial (NCT04334759) which compares durvalumab with chemotherapy to chemotherapy alone¹⁵. Of note, the survival for patients with epithelioid MPM in the PrE0505 trial exceeded two years and several patients with epithelioid MPM continue to be free from tumor progression at the time of this publication. This potential benefit from chemo-immunotherapy for epithelioid MPM is in contrast to the recent CheckMate-743 trial that reported a striking survival advantage favoring ipilimumab-nivolumab over chemotherapy for patients with non-epithelioid histology (18.1 versus 8.8 months); however no significant survival difference between the two treatment arms for patients with epithelioid MPM (18.7 versus 16.5 months)¹⁶. Given the known chemosensitivity of epithelioid MPM and relative chemo-resistance of non-epithelioid MPM it is possible that chemo-immunotherapy may confer a synergistic advantage particularly for patients with epithelioid MPM. As both DREAM and PrE0505 trials mandated that patients conclude durvalumab treatment after one full year of treatment, it is conceivable that some patients would derive additional benefit from maintenance therapy until disease progression, although data on this point are conflicting across tumor types³⁰. The investigational arm of the ongoing phase 3 PrE0506/DREAM3R trial includes treatment with maintenance durvalumab until confirmed disease progression thus addressing this potential concern.

The clinical efficacy of chemo-immunotherapy demonstrated in the PrE0505 trial challenged the common paradigm of immunotherapy responsive tumors, as MPM harbors a low nonsynonymous TMB that conceptually may limit the number of presented immunogenic neoantigens. While tumors with TMB in the lower end of the spectrum are historically thought to have TMB-independent mechanisms of response to immunotherapy¹⁹, we discovered that a higher immunogenic mutation load distinguished responding tumors, particularly in the epithelioid MPM group. Importantly, these findings were not corroborated in MPM treated with standard-of-care therapies, suggesting an association with durvalumab. Clonal TMB represents a dominant tumor-intrinsic determinant of clinical response to immunotherapy³¹, which is consistent with our findings of clonal TMB predicting radiographic response in epithelioid MPM. Similarly, a high subclonal mutation burden, in part mediated by abnormal activity of the APOBEC enzymes; may enable tumor immune escape^{32,33}. We indeed discovered an inverse association between an APOBEC mutational signature and response to chemo-immunotherapy in epithelioid MPM in the PrE0505 cohort. To substantiate these findings, we pulsed autologous T cells with peptides derived from immunogenic mutations and identified neoantigen-specific TCR expansions in vitro, suggesting that robust neoantigen-specific responses were linked with favorable clinical outcome.

Consistent with the notion that MPM is driven by inactivating mutations in tumor suppressor genes17,18,29,34,35, we identified recurring inactivating largely non-overlapping genomic alterations in BAP1, CDKN2A, NF2, SETD2, PBRM1 and TP53 independent of therapeutic response. While we did not find an enrichment in alterations of any single gene in tumors from patients with differential responses to chemo-immunotherapy, a trend towards an enrichment in somatic mutations in chromatin regulating genes was noted in tumors from patients achieving an overall survival ≥12 months; these alterations may mediate transcriptional changes of genes involved in immune related signaling pathways³⁶ or may be linked with a genomic instability phenotype that can predispose to response to immunotherapy³⁷.

Germline genetic susceptibility has been established as a seminal event in MPM tumorigenesis, mostly involving tumor suppressor genes in DNA repair mechanisms^{20,22,29,38} The frequency of 32% for BAP1 genomic alterations in the PrE0505 cohort is in line with these previously reported analyses^{17,29,39} Presence of germline BAP1 mutations has been linked with a longer 5-year survival, suggesting a less aggressive phenotype of MPM arising in the context of a BAP1 cancer syndrome^{28,39,40}. The underlying etiology of this phenomenon remains unclear, with one potential explanation being that the tumor microenvironment of BAP1-null tumors is more inflammatory²⁷. While we did not find prolonged survival for patients with somatic BAP1 mutations, BAP/mutant tumors were found to have a higher degree of CD8+ T cell infiltration. Importantly, patients harboring deleterious germline mutations in MPM predisposing genes, including but not limited to genes involved in DNA homologous recombination, achieved significantly longer progression-free and overall survival with chemo-immunotherapy. Inherited defects in homologous recombination repair, resulting in microdeletions and DNA breaks, may be linked with longer overall survival following platinum chemotherapy^{20,28} as well as affect adaptive and innate immunity, ultimately potentiating response to immune checkpoint blockade^{41,42}. Our findings suggest that germline genotypes may impact clinical outcomes after chemo-immunotherapy and germline testing should be considered for clinical decision making for patients with mesothelioma.

Importantly, we found that DNA repair deficiency and defective homologous recombination in particular, determined by both sequence mutational spectra as well as by genome-wide copy number analyses was a hallmark of responding tumors, especially for epithelioid MPM. Overall, responding epithelioid tumors harbored a higher content of genome-wide copy number breakpoints, suggesting that genomic instability impacts therapeutic efficacy for chemo-immunotherapy. While we did not detect any evidence of oscillating copy number changes within any given chromosome indicative of chromothripsis in the PrE0505 cohort, there were 3 cases with extensive genome-wide loss of heterozygosity, a phenomenon called genome near-haploidization (GNH) which has been previously reported in five MPM cases¹⁷. Interestingly, two of the tumors with GNH in our cohort harbored BAP1 mutations and all patients achieved an overall survival longer than 12 months. As GNH harboring MPM may comprise a novel molecular subtype of MPM with distinctive clinical behavior¹⁷, our findings suggest that these unique genomic features may be linked with favorable response to chemo-immunotherapy. Conceptually, immunogenic mutations residing in genomic loci that undergo haploidization cannot be lost under the selective pressure of immunotherapy⁴³, and therefore may drive a sustained anti-tumor immune response. Consistent with this hypothesis, we discovered that tumors that harbored a higher number of sequence alterations in single copy regions of the genome responded to combined chemo-immunotherapy.

The density of the CD8+ T cell infiltrate has been associated with effective anti-tumor immune responses^{44,45}, however in the PrE0505 cohort, neither CD8+ T cell infiltration nor PD-L1 protein expression predicted response to chemo-immunotherapy. Notably, the tumor microenvironment of responding tumors contained a less clonal T cell receptor repertoire that become more polarized at the time of acquired resistance. In contrast to the notion that anti-tumor immune responses in the context of immunotherapy are driven by a clonal T cell repertoire in TMB-high tumors such as melanoma⁴⁴ or non-small cell lung cancer⁴⁶, our findings suggest that maximal immune cell repertoire diversity is required to mount an effective anti-tumor immune response in MPM. Consistent with this notion, a higher TCR diversity has been previously shown to correlate with improved outcome in bladder cancer, colorectal cancer, hepatocellular carcinoma and uterine cancer⁴⁷.

Our study was limited by its small sample size and absence of a non-durvalumab control arm. In order to interpret our molecular findings with respect to response to chemo-immunotherapy compared to standard-of-care therapy alone, we performed genomic analyses of an independent cohort of 82 mesotheliomas obtained from the TCGA registry. This cohort did not include patients treated with chemo-immunotherapy or immunotherapy and analyses of the TCGA mesothelioma cohort suggested that the genomic features of response in the PrE0505 trial were driven by durvalumab. Importantly, definitively assessing the predictive versus prognostic nature of our findings is needed and planned within the ongoing phase 3 randomized DREAM3R/PrE0506 trial. Furthermore, while the control survival was chosen based on the historical control that led to the approval of pemetrexed with cisplatin⁵, recent randomized data have shown both shorter and longer survival for the pemetrexed-cisplatin combination^{8,9}, it is therefore possible that control assumptions may have underestimated the expected survival with chemotherapy alone.

In summary, we report the favorable clinical efficacy of the PrE0505 study of chemo-immunotherapy for unresectable MPM with in depth molecular and functional analyses, which provide an understanding of the complex genomic and immune cell features of response to combined chemo-immunotherapy with potential broad clinical implications.

TABLE 1
Tables
Demographic characteristics of study participants.

| Characteristic | Number (Total N = 55) | Range or (%) |
|---|---|---|
| Age (years) | 68 (median) | 35-83 |

Sex

| | | |
|---|---|---|
| Female | 10 | (18.2) |
| Male | 45 | (81.8) |
| Ethnicity | | |
| Caucasian | 47 | (85.5) |
| African-American | 2 | (3.6) |
| Other | 6 | (10.9) |

ECOG Performance Status

| | | |
|---|---|---|
| 0 | 23 | (41.8) |
| 1 | 32 | (58.2) |

Histologic Subtype

| | | |
|---|---|---|
| Epithelioid | 41 | (74.5) |
| Sarcomatoid | 7 | (12.7) |
| Biphasic | 6 | (10.9) |
| Desmoplastic | 1 | (1.8) |

### Example 1

### Methods

### Trial Design, Endpoints, Oversight and Samples Analyzed.

The first patient enrollment date was: Jun. 12, 2017 and the last patient enrollment date was: Jun. 21, 2018 and a detailed outline of the numbers of patients available for analyses. Due to biospecimen availability and biospecimen quality, germline genomic data were evaluable for 44 patients and somatic genomic data were evaluable for 40 patients. A Data Safety Monitoring Board reviewed the study twice a year and patient consent was provided for sample collection.

The full list of the inclusion and exclusion criteria is shown below:
Histologically and/or cytologically confirmed malignant pleural mesothelioma.
Unresectable disease (defined as the participant not being a candidate for curative surgery).
Measurable disease, defined as at least 1 lesion (measurable) that can be accurately assessed at baseline by computed tomography (CT) or magnetic resonance imaging (MRI) and is suitable for repeated assessment (modified RECIST for pleural mesothelioma).
Available unstained archived tumor tissue sample in sufficient quantity to allow for analyses. At least fifteen unstained slides or a tumor block (preferred). NOTE: A fine needle aspiration sample is not sufficient to make the patient eligible for enrollment. Given the complexity of mesothelioma pathological diagnosis and that these will be newly diagnosed patients it is expected that they will have a core needle biopsy or surgical tumor biopsy as part of their initial diagnostic work up.
Age ≥18 years.
Eastern Cooperative Oncology Group (ECOG) performance status of 0-1.
Ability to understand and willingness to sign Institutional Review Board (IRB)-approved informed consent.
Willing to provide archived tumor tissue and blood samples for research.
Adequate organ function as measured by the following criteria, obtained ≤2 weeks prior to registration:
   Absolute Neutrophil Count (ANC)≥1500/mm³
   Hemoglobin >9.0 g/DI
   Platelets >100,000/mm³
   Serum creatinine clearance (CL)>60 mL/min by the Cockcroft-Gault formula or by 24-hour urine collection for determination of creatinine clearance. NOTE: Patients with a creatinine Cl≥45 mL/min however ≤60 mL/min may be considered for enrollment provided they fulfill all other eligibility criteria, these subjects will receive pemetrexed carboplatin concurrent with durvalumab during the combination phase of treatment. Patients with a creatinine CL<45 mL/min should not be enrolled.
   Albumin ≥2.8 g/dL
   Total Bilirubin≤1.5× Upper Limit of Normal (ULN)
   Aspartate Aminotransferase (AST)/Alanine Aminotransferase (ALT)≤2.5×ULN (≤5×ULN in patients with liver metastases)
Women must either be of non-reproductive potential or must have a negative serum pregnancy test upon study entry.
Women must not be pregnant or breastfeeding.
Patient is willing and able to comply with the protocol for the duration of the study including undergoing treatment and scheduled visits and examinations including follow-up.
Patient must not have involvement in the planning and/or conduct of the study. No previous enrollment in the present study.
Patients may not have participated in another clinical study with an investigational product during the last 4 weeks.
Patients must not have any prior systemic therapy (chemotherapy, immunotherapy, endocrine therapy, targeted therapy, biologic therapy, tumor embolization, monoclonal antibodies, and other investigational agent) for mesothelioma.
No previous treatment with a PD1 or PD-L1 inhibitor, including durvalumab or any other agent targeting immune checkpoints.
Patients must not have non-pleural mesothelioma e.g. mesothelioma arising in peritoneum, tunica vaginalis or any serosal surface other than the pleura.
Patients must not have an active second malignancy other than non-melanoma skin cancer or cervical carcinoma in situ.
Patients must not have mean QT interval corrected for heart rate (QTc)≥470 ms calculated from 3 electrocardiograms (ECGs) using Frediricia's Correction.
Patients must not have symptomatic or uncontrolled brain metastases requiring concurrent treatment, inclusive of but not limited to surgery, radiation and/or corticosteroids (prednisone >10 mg or equivalent). Surgery, radiation and/or corticosteroids (any dose >10 mg prednisone equivalent) must have been completed ≥2 weeks prior to registration.
Patients must not have uncontrolled seizures.
Patients must not have current or prior use of immunosuppressive medication within 28 days before the first dose of durvalumab, with the exceptions of intranasal and inhaled corticosteroids or systemic corticosteroids at physiological doses, which are not to exceed 10 mg/day of prednisone, or an equivalent corticosteroid. Standard steroid premedication given prior to chemotherapy or as prophylaxis for imaging contrast allergy should not be counted for this criterion.
No active or prior documented autoimmune or inflammatory disorders (including inflammatory bowel disease, diverticulitis with the exception of diverticulosis, celiac disease, irritable bowel disease; Wegner syndrome) within the past 2 years. Subjects with vitiligo, alopecia, Grave's disease, or psoriasis not requiring systemic treatment (within the past 3 years) are not excluded.
No history of primary immunodeficiency.
No history of allogeneic organ transplant.
No history of hypersensitivity to durvalumab, cisplatin, carboplatin, pemetrexed or any of their excipients.
No uncontrolled intercurrent illness including, but not limited to, ongoing or active infection, symptomatic congestive heart failure, uncontrolled hypertension, unstable angina pectoris, cardiac arrhythmia, active peptic ulcer disease or gastritis, active bleeding diatheses including any subject known to have psychiatric illness/social situations that would limit compliance with study requirements or compromise the ability of the subject to give written informed consent.
No active infection including tuberculosis (clinical evaluation including: physical examination findings, radiographic findings, positive PPD test, etc.), hepatitis B (known positive HBV surface antigen [HBsAg] result), hepatitis C, or human immunodeficiency virus (positive HIV 1/2 antibodies as defined by a positive ELISA test). Patients with a past or resolved HBV infection (defined as the presence of hepatitis B core antibody [anti-HBc] and absence of HBsAg) are eligible. Patients positive for hepatitis C (HCV) antibody are eligible only if polymerase chain reaction is negative for HCV RNA. HIV testing is not required in absence of clinical suspicion.
No known history of leptomeningeal carcinomatosis.
Patients must not have received live attenuated vaccination within 30 days prior to study entry or within 30 days of receiving durvalumab.
Patients must not have any condition that, in the opinion of the investigator, would interfere with evaluation of study treatment or interpretation of patient safety or study results.

Imaging was performed every 6 weeks during the concurrent phase of treatment and every 9 weeks during maintenance durvalumab. Best objective response was evaluated by RECIST Version 1.1 criteria modified for mesothelioma. Toxicity was determined using the CTCAE Version 4.03 criteria.

Secondary objectives included safety and tolerability of durvalumab and durvalumab in combination with chemotherapy in subjects with malignant pleural mesothelioma, percentage of patients progression-free at 24 weeks from the time of registration (response coded based on modified RECIST 1.1 criteria for mesothelioma), progression-free survival-measured from the time of study registration until radiologic progression, clinical progression or death, best objective response rate with evaluation continued up to 1 year (response coded based on modified RECIST Version 1.1 criteria for mesothelioma). Exploratory objectives included assessment of tumor baseline PD-L1 expression, the genomic and neoantigen landscape of tumors, dynamics of circulating cell free tumor DNA and other blood-based biomarkers.

### Whole Exome Sequencing

### TCGA Mesothelioma Cohort

We obtained matched tumor-normal exome sequencing data from 82 patients with MPM in TCGA (cancergenome.nih.gov), as outlined in the TCGA publication guidelines cancergenome. nih.gov/publications/publicationguidelines. WES-derived somatic mutation calls from the TCGA PanCancer Atlas MC3 project were retrieved from the NCI Genomic Data Commons (gdc.cancer.gov/about-data/publications/mc3-2017). The MC3 mutation call set is the result of application of a uniform analysis pipeline including a standardized set of six mutation callers and an array of automated filters to all the entire TCGA exome data⁴⁸. Tumor mutation burden was calculated as the number of nonsynonymous mutations detected by whole exome sequencing.

### Tissue Sample Characteristics and Sample Preparation

Formalin fixed paraffin embedded tumor tissue and matched peripheral blood were collected prior to therapy initiation. DNA was extracted from patients' tumors and matched peripheral blood using the Qiagen DNA kit (Qiagen, CA). Fragmented genomic DNA from tumor and normal samples was used for Illumina TruSeq library construction (Illumina, San Diego, CA) and exonic regions were captured in solution using the Agilent SureSelect v.4 kit (Agilent, Santa Clara, CA) according to the manufacturers' instructions as previously described^{43,49,50}. Paired-end sequencing, resulting in 100 bases from each end of the fragments for the exome libraries was performed using Illumina HiSeq 2000/2500 instrumentation (Illumina, San Diego, CA). The mean depth of total coverage for the pre-treatment tumors and matched normal DNA samples was 220× (166× distinct) and 105× (90× distinct) respectively. On average, 94% of the bases in the target region had a minimum coverage of 10×; four tumor samples (329, 351, 629, and 923) were determined to be of low purity by mutation and copy number analyses and were excluded from all WES-based analyses of somatic alterations, while their matched normal DNA samples were included in the germline analyses.

### Somatic Mutation Calling, Immmogenic Mutation Characterization and Neoantigen Prediction

Somatic mutations, consisting of point mutations, insertions, and deletions across the whole exome were identified using the VariantDx custom software for identifying mutations in matched tumor and normal samples as previously described^{43,49}. Mutations were annotated with the number of tumor samples harboring identical amino acid changes in cosmic database (v91). MHC class I and II neoantigens were derived from nonsynonymous single base substitutions using MHCnuggets⁵¹. Ranks of neopeptides were determined based on their MHC binding affinity compared to 10,000 human proteome peptides per peptide length per binding MHC allele. Sequence alterations resulting in neopeptides ranking in the 1^{st} percentile were considered putatively immunogenic mutations.

### Germline Predisposition Characterization

A set of cancer susceptibility genes with alterations contributing to germline predisposition to mesothelioma was compiled from the literature³. Nonsynonymous germline alterations in the above set were identified by applying Strelka 2.9.2⁵² and the candidate mutation set was first filtered to include positions where the genotype was of sufficient quality and could be resolved in both normal and tumor samples of each patient. Variants were subsequently annotated using OpenCravat⁵³. Confirmed pathogenic variants-hereafter termed germline deleterious mutations-including nonsense, frameshift, splice site, and missense variants, in genes with known cancer susceptibility potential were identified based on annotation in the Clin Var database and published evidence of a damaging effect on protein function.

### Mutation Signatures

Mutation signatures were derived based on the fraction of coding point mutations in each of 96 trinucleotide contexts and estimated the contribution of each signature to each tumor sample using the deconstructSigs R package (v1.8.0) with the default “signatures. nature2013” settings^{54,55}.

### HLA Germline and Somatic Analyses

OptiType v1.2. was used to determine HLA class I haplotypes⁵⁶, xHLA was used to determine HLA class II haplotypes for HLA-DPB1, HLA-DQB1, HLA-DRB1⁵⁷, and SOAP-HLA was used to determined class II haplotypes for HLA-DPA1 and HLA-DQA1⁵⁸. A separate bioinformatic analysis using POLYSOLVER⁵⁹ was utilized to detect and annotate the somatic mutations in class I HLA genes. We determined HLA class I loss in the tumor by applying LOHHLA⁶⁰. We evaluated somatic loss of HLA class II genes by review of allele-specific copy number of these loci, where minor copy number of zero indicated loss of heterozygosity. The number of unique tumor HLA class I and II alleles was calculated by subtracting the number of heterozygous alleles with somatic LOH from the total number of unique germline alleles. We subsequently computed an HLA Evolutionary Divergence (HED) score by using Grantham distances between protein sequences of allele pairs for each HLA-A, HLA-B and HLA-C locus²⁵. HLA class I allele protein sequences are obtained from the ImMunoGeneTics/HLA database⁶¹. A cumulative HED score for each sample was also computed as the arithmetic mean of the three individual divergences, assuming equal contribution from each locus.

### Genome-Wide Copy Number Analyses

We utilized FACETS 0.6.1 to estimate the purity of each tumor sample, the integer allele-specific copy number profile across the genome, and the cellular fraction associated with each aberrant somatic copy number alteration⁶². The estimated allele-specific copy number profiles were reviewed to ensure quality of fit. In four cases with very low tumor purity (329, 351, 629, and 923), the copy number states and ploidy could not be resolved; these cases were excluded from subsequent copy number based analyses. Furthermore, we investigated potential associations between copy number-derived tumor purity and tumor mutation and immunogenic mutation load; these analyses revealed a weak association between tumor purity and tumor mutation burden derived features when all patients were considered, but no statistically significant association between these features in epithelioid mesotheliomas. Three cases (225, 926 and 922) harbored extensive loss of heterozygosity across the genome with evidence of genome near-haploidization (FIG. 4). In each tumor sample, the number of sequence alterations overlapping loci with total copy number of 1 was recorded. Focal copy number changes, i.e. amplifications and deletions, were determined as genomic regions of a size smaller than 30 Mb where the assigned copy number was zero (homozygous deletion), or it exceeded three times the ploidy of the tumor sample (amplifications). The estimated ploidy was rounded to the closest integer level, and was used as the reference for determining loss, gain, or neutral status of each copy number segment. Gain, loss, or loss of heterozygosity in each chromosome arm was evaluated if at least 90% of the length of the arm was covered by segments of the given status. The statistical significance of the prevalence of each category of alterations across the arms was evaluated by performing a permutation experiment. In this experiment, each permutation sample was a vector of size 39 where the total number of chromosome arms harboring gain, loss, or LOH equaled this value for one of the samples in the main cohort to match the observed level of aneuploidy in the population.

### Mutation Clonality Estimation

For each somatic sequence alteration, the observed mutant and total read counts, the tumor purity and the tumor copy number at the mutated locus were integrated using SCHISM⁶³ and as previously described⁵⁰ to determine the clonality, i.e. the fraction of cancer cells that harbor the alteration.

### Aneuploidy Assessment

Several metrics characterizing the degree of genome aneuploidy were calculated including the fraction of genome with loss of heterozygosity (LOH), the fraction of genome with allelic imbalance, the number of copy number breakpoints, and the entropy of the multinomial probability distribution corresponding to the genome representation of different copy number levels⁵⁰. The number of copy number breakpoints was used as a proxy measure for the extent of somatic structural alterations in each tumor.

### Homologous Recombination Deficiency Estimation

To assess the extent of homologous recombination deficiency in tumors, three individual and one combined metric were determined based on the allele-specific copy number profiles by applying the R package scar-HRD 0.1.1⁶⁴: telomeric allelic imbalance (HRD-TAI score; the count of CN segments with allelic imbalance that extend of telomeres), loss of heterozygosity profiles (HRD-LOH score; the number of segments with a minimum size of 15 Mb which do not span the entire chromosome), and large-scale state transitions (HRD-LST score; the number of breakpoints between segments with minimum size of 10 Mb where the gap between the segments does not exceed 3 Mb). A combined metric for homologous recombination deficiency, HRD-sum, was defined as the sum of the three individual metrics.

### Evaluation of the Background Rate of Genomic Loss

To better characterize the background rate of loss in regions of the genome with a single copy per cell (haploid) versus euploid regions (2 copies per cell, no loss of heterozygosity), we analyzed somatic copy number profiles of 1086 mesothelioma and non-small cell lung cancer tumors from TCGA. In each tumor, we first determined the chromosome arms where at least 75% of the arm length was covered by the copy number state of interest. The set of tumor samples was then narrowed down to those with at least one arm in haploid state (n=544). Next, across all chromosome arms of a given state, the rate of loss was determined as follows: in the haploid arms, the loss rate was defined as the total number of bases with somatic copy number of 0 within these arms, divided by the total length of arms in haploid state. For the diploid arms, the loss rate was defined as the total number of bases with somatic copy number of 0 within these arms multiplied by 2 added to the number of bases with somatic copy number of 1, and then divided by the total length of arms in euploid state.

### TCR Sequencing

Intra-tumoral TCR clones were evaluated by next generation sequencing of the baseline tumor as well as matched baseline-resistant tumors for cases 295, 459 and 926. TCR-13 CDR3 regions were amplified using the survey ImmunoSeq assay in a multiplex PCR method using 45 forward primers specific to TCR V13 gene segments and 13 reverse primers specific to TCR J13 gene segments (Adaptive Biotechnologies)⁶⁵. Productive TCR sequences were further analyzed and clone counts were based on CDR3 amino acid sequences. Dominant TCR clones were assessed by estimating the proportion of TCR repertoire constituted by the top 5% of unique clones, for these analyses TCR repertoires were filtered for clones representing at least 0.01% of the repertoire. For each sample, a clonality metric was estimated in order to quantitate the extent of mono- or oligo-clonal expansion by measuring the shape of the clone frequency distribution. For differential abundance analysis between baseline and on-therapy tumors, we selected the most expanded and most regressed TCR clonotypes, corresponding to fold changes in productive frequency of TCR clones with an FDR<0.0101 (Fisher's Exact test) and requiring at least 0.01% relative repertoire abundance at baseline or resistance time-points.

### RNA Sequencing

Total RNA was extracted from 10 μm FFPE sections with the RNeasy FFPE kit (Qiagen). The quality of total RNA was assessed by calculating the DV200 index measured with the RNA 6000 Pico Kit (Agilent Technologies). RNAseq libraries were generated by ribosomal depletion (Illumina Ribo-Zero Gold IRNA removal kit) followed by reverse transcription into strand-specific cDNA libraries (NEBNext Ultra directional RNA library kit for Illumina). Paired-end sequencing, resulting in 150 bases from each end of the fragments, was performed using Illumina NovaSeq 6000 S4 generating an average of 200M total reads per library. RNA-seq data was then mapped to the human transcriptome using STAR⁶⁶ followed by RSEM for isoform and gene-level quantification⁶⁷. Transcripts associated with RNA genes, mitochondrial genes, and ribosomal proteins were masked Normalization of raw transcript counts and differential expression analysis was performed using DESeq2⁶⁸.

### PD-L1 and CD8 Immunohistochemistry

Immunohistochemistry for CD8/PD-L1 dual detection was performed on formalin-fixed, paraffin embedded sections on a Ventana Discovery Ultra autostainer (Roche Diagnostics) utilizing a primary mouse anti-human CD8 antibody, (1:100 dilution, clone m7103, Dako) and a rabbit anti-human anti-PD-L1 antibody (1:100 dilution; EIL3N clone, Cell Signaling Technologies) as previously described. A minimum of 100 tumor cells were evaluated per specimen and a PD-L1 tumor proportion score (TPS) was calculated based on the percentage of tumor cells with PD-L1 positive staining. CD8-positive lymphocyte density was evaluated by the average number of CD8+ cells in 10 representative high power fields (40× objective, 400× magnification).

### Functional T Cell Assays

To identify immunogenic mutation-derived neopeptide-specific, HLA class I restricted T cell clones in the peripheral blood, we applied the high throughput TCRseq-based platform MANAFEST (Mutation Associated NeoAntigen Functional Expansion of Specific T-cells) as previously described^{43,69}. Briefly, putative neopeptides identified above (jpt Peptide Technologies) were each used to stimulate 250,000 T cells in vitro for 10 days. On day 0, T cells were isolated from peripheral blood mononuclear cells (PBMC) by negative selection (EasySep; STEMCELL Technologies). The T cell-negative fraction was co-cultured with an equal number of selected T cells in culture medium (IMDM/5% human serum with 50 μg/mL gentamicin) with 1 μg/mL relevant neoantigenic peptide, 1 μg/mL of an MHC class I-restricted CMV, EBV, and flu peptide epitope pool (CEFX, jpt Peptide Technologies), 1 μg/mL of pools representing the HIV-1 Gag protein (ipt Peptide Technologies) and no peptide. On day 3, half the medium was replaced with fresh medium containing cytokines for a final concentration of 50 IU/ml IL-2 (Chiron), 25 ng/ml IL-7 (Miltenyi), and 25 ng/ml IL-15 (PeproTech). On day 7, half the medium was replaced with fresh culture medium containing cytokines for a final concentration of 100 IU/mL IL-2 and 25 ng/ml IL-7 and IL-15. On day 10, cells were harvested, washed twice with PBS, and the CD8+ fraction was isolated using a CD8+ negative enrichment kit (EasySep; STEMCELL Technologies). DNA was extracted from each CD8-enriched culture condition. TCR Vβ CDR3 sequencing was performed by the SKCCC FEST and TCR Immunogenomics Core (FTIC) on genomic DNA from each T cell condition using the Oncomine TCR Beta short-read assay (Illumina, Inc). DNA libraries were pooled and sequenced on an Illumina iSeq 100 using unique dual indexes to prevent index hopping, with an estimated recovery of ˜50,000 reads per sample. Data pre-processing was performed to eliminate non-productive TCR sequences (sequences that did not translate into a productive protein) and to align and trim the nucleotide sequences to obtain only the CDR3 region. Additionally, for inclusion in our analyses, CDR3 sequences needed to begin with “C”, end with “F” or “W”, and have at least 7 amino acids in the CDR3, which are universally accepted parameters for delineating the CDR3 region⁷⁰. Productive clonality of each sample and productive frequency of each clone was calculated to reflect the processed data. Resultant processed data files were uploaded to our publicly-available MANAFEST analysis web app (www.stat-apps.one.jhmi.edu) to bioinformatically identify neoantigen-specific T cell clonotypes. To be considered antigen-specific, a T-cell clonotype must have met the following criteria: 1) significant expansion (Fisher's exact test with Benjamini-Hochberg correction for FDR, p<0.05) compared to T cells cultured without peptide, 2) significant expansion compared to every other peptide-stimulated culture (FDR<0.05), 3) an odds ratio >5 compared to all other conditions, 4) at least 30 reads in the “positive” well, and 4) at least 2× higher frequency than background clonotypic expansions as detected in the HIV negative control condition

### Statistical Analyses

OS and PFS distributions were estimated using the Kaplan-Meier method, and Cox proportional hazards models were used to estimate the hazard ratios among subgroups. The confidence intervals of objective response rate (defined as the percentage of patients achieving complete or partial response) we calculated based on an exact binomial distribution. Objective response rates were compared between subgroups using Fisher's exact tests. Differences in genomic and molecular features between tumors of responding and non-responding patients were evaluated using chi-squared or Fisher's exact test for categorical variables and the Mann-Whitney test for continuous variables. The Pearson correlation coefficient (R) was used to assess correlations between continuous variables and the Spearman rho coefficient was calculated for non-parametric correlations. We investigated potential correlations between the genomic features described and other than the expected co-linearity between non-synonymous mutation burden and MHC class I and II mutation associated neoantigens we did not identify any potential confounding relationships among features. The median point estimate and 95% CI for progression-free and overall survival were estimated by the Kaplan-Meier method and survival curves were compared by using the nonparametric log rank test. For the survival analyses of the TCGA mesothelioma cohort, progression-free interval was defined as the time interval from diagnosis to progression of disease, local recurrence, distant metastasis or death, whichever was applicable. Statistical analyses were done using the SPSS software program (version 25.0.0 for Windows, IBM, Armonk, NY), SAS (version 9.4) and R version 3.2 and higher (cran.r-project.org).

### Data Availability Statement

All raw sequencing data, utilized to generate FIGS. 2-5 have been deposited in the European Genome-phenome Archive (EGA accession number EGAS00001005426). Source data for FIGS. 1-5 are provided with the paper and in Source Data files. Source data for the TCGA tumor samples were retrieved from cancergenome.nih.gov. WES-derived somatic mutation calls from the TCGA PanCancer Atlas MC3 project were retrieved from the NCI Genomic Data Commons (gdc.cancer.gov/about-data/publications/mc3-2017).

### Example 2

We have recently shown that a higher number of sequence alterations contained in single copy regions of the genome differentiate responding from non-responding tumors in the context of immune checkpoint blockade (Forde et al., Durvalumab with Platinum-Pemetrexed for Unresectable Pleural Mesothelioma: Survival, Genomic and Immunologic Analyses from the phase 2 PrE0505 trial Nature medicine (2021)). These findings suggested that mutations and associated neoantigens contained in regions of the genome with in a single copy per cancer cell cannot be eliminated under the selective pressure of therapy and therefore mediate sustained neoantigen-driven immune responses and long-term clinical benefit.

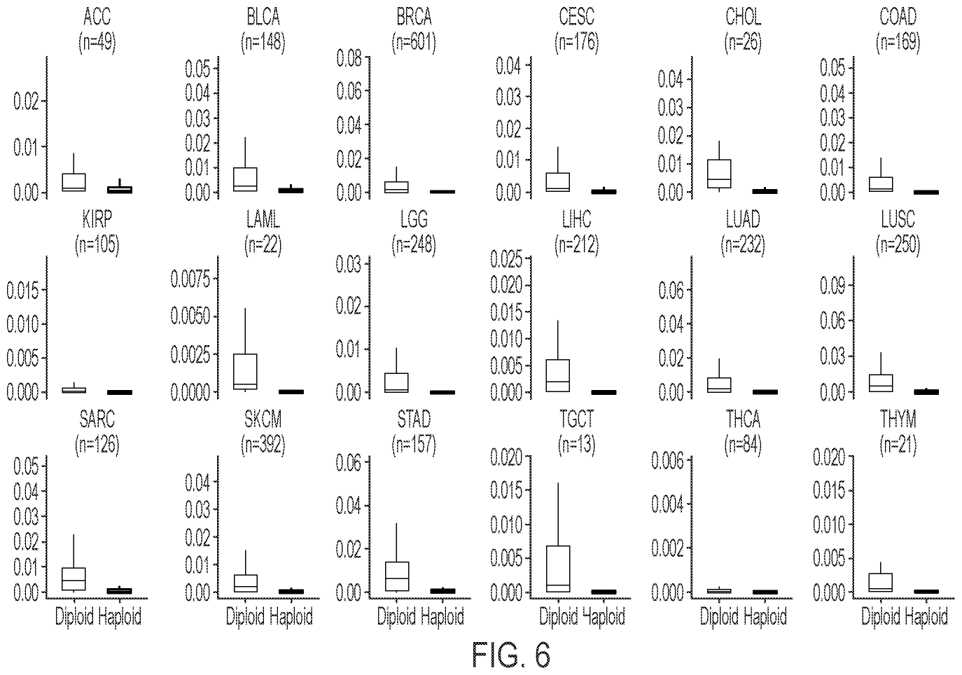

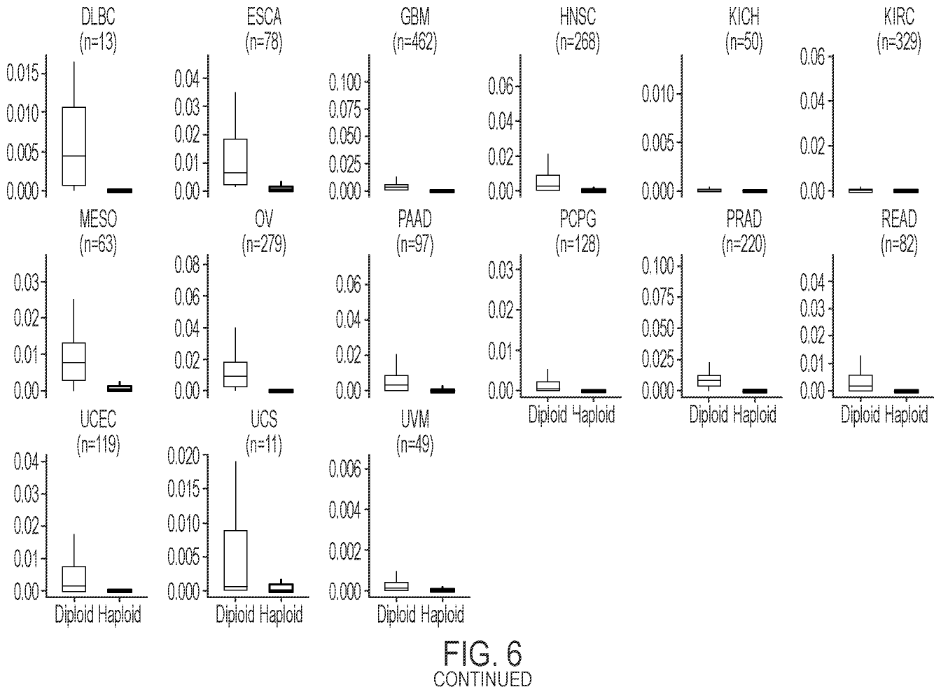

To further support these findings in a pan-cancer manner, we investigated the background rate of loss in regions of the genome with a single copy per cell (haploid) versus euploid regions (2 copies per cell) and analyzed somatic copy number profiles of 5,279 tumors, including immunotherapy responsive cancers such as melanoma, non-small cell lung cancer and mesothelioma. These analyses revealed that the rate of loss in haploid regions was consistently lower than that of euploid regions (FIG. 6), supporting the notion that mutations contained in these regions are hard to eliminate and may drive a sustained anti-tumor immune response.

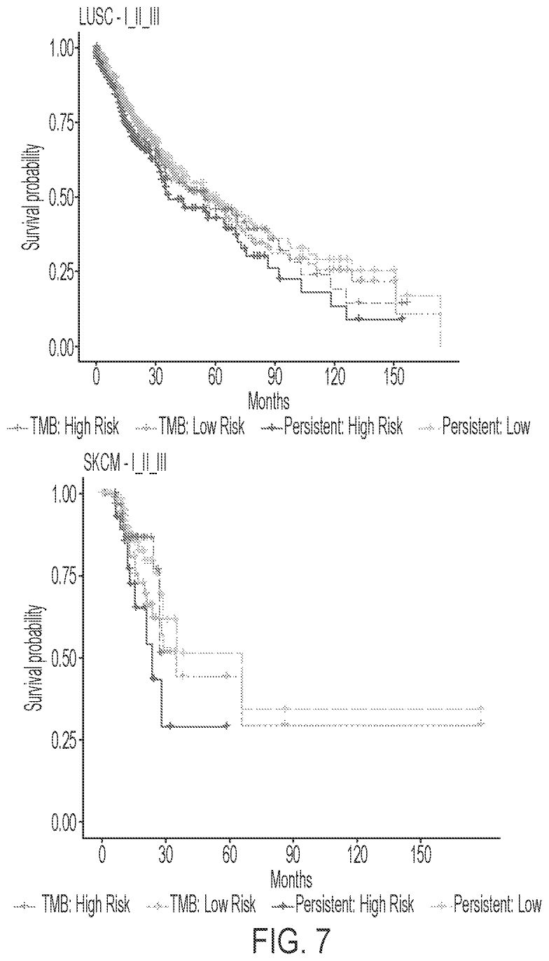

Conceptually, mutations contained in single or multiple copies of the tumor genome cannot be lost under the selective pressure of immunotherapy (Anagnostou, V. et al. Evolution of Neoantigen Landscape during Immune Checkpoint Blockade in Non-Small Cell Lung Cancer. Cancer Discov 7, 264-276, doi:10.1158/2159-8290.CD-16-0828 (2017)), and therefore may drive a sustained anti-tumor immune response. This persistent mutation burden essentially functions as an intrinsic vaccine that fuels adaptive immune responses in the tumor microenvironment and cannot be by passed by neoantigen loss via chromosomal deletions and loss of heterozygosity. Consistent with this hypothesis, patients with tumors with a higher number of sequence alterations in single or multiple copy regions of the cancer genome (persistent mutation burden) had a longer overall survival, in analyses of the TCGA non-small cell lung cancer and melanoma sub-cohorts (FIG. 7).

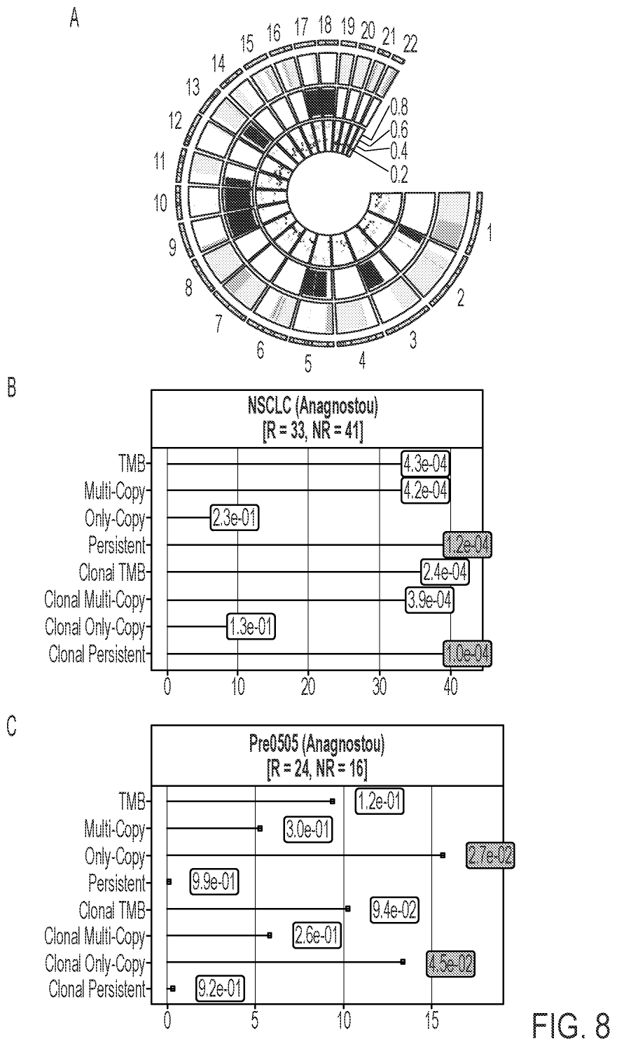

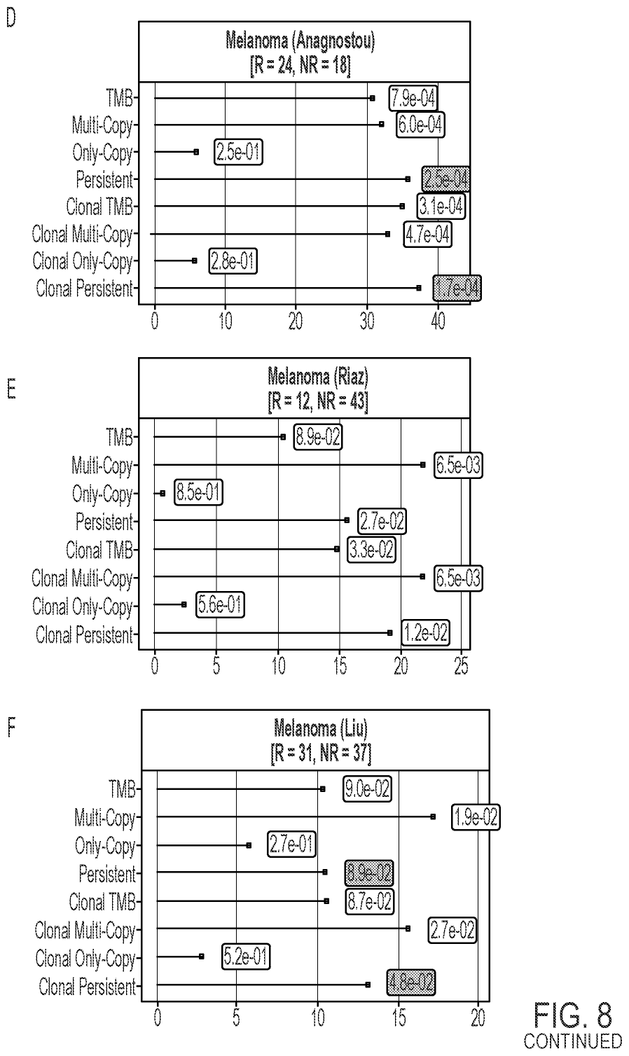

Importantly, we discovered that tumors with a high persistent mutation burden were more responsive to immune checkpoint blockade compare to TMB-high tumors in a series of cohorts spanning non-small cell lung cancer, melanoma and mesothelioma and various immune checkpoint inhibitors (FIG. 8).

### REFERENCES

1 Odgerel, C. O. et al. Estimation of the global burden of mesothelioma deaths from incomplete national mortality data. Occupational and environmental medicine 74, 851-858, doi:10.1136/oemed-2017-104298 (2017).
2 Tweedale, G. Asbestos and its lethal legacy. Nature reviews. Cancer 2, 311-315, doi:10.1038/nrc774 (2002).
3 Panou, V. et al. Frequency of Germline Mutations in Cancer Susceptibility Genes in Malignant Mesothelioma. Journal of clinical oncology: official journal of the American Society of Clinical Oncology 36, 2863-2871, doi:10.1200/JCO.2018.78.5204 (2018).
4 Carbone, M. et al. Tumour predisposition and cancer syndromes as models to study gene-environment interactions. Nature reviews. Cancer 20, 533-549, doi:10.1038/s41568-020-0265-y (2020).
5 Vogelzang, N. J. et al. Phase III study of pemetrexed in combination with cisplatin versus cisplatin alone in patients with malignant pleural mesothelioma. Journal of clinical oncology: official journal of the American Society of Clinical Oncology 21, 2636-2644, doi:10.1200/JCO.2003.11.136 (2003).
6 Krug, L. M. et al. Randomized phase II trial of pemetrexed/cisplatin with or without CBP501 in patients with advanced malignant pleural mesothelioma. Lung cancer 85, 429-434, doi:10.1016/j.lungcan.2014.06.008 (2014).
7 Scagliotti, G. V. et al. Nintedanib in combination with pemetrexed and cisplatin for chemotherapy-naive patients with advanced malignant pleural mesothelioma (LUME-Meso): a double-blind, randomised, placebo-controlled phase 3 trial. The Lancet. Respiratory medicine 7, 569-580, doi:10.1016/S2213-2600(19)30139-0 (2019).
8 Tsao, A. S. et al. Phase II Trial of Cediranib in Combination With Cisplatin and Pemetrexed in Chemotherapy-Naive Patients With Unresectable Malignant Pleural Mesothelioma (SWOG S0905). Journal of clinical oncology: official journal of the American Society of Clinical Oncology 37, 2537-2547, doi:10.1200/JCO.19.00269 (2019).
9 Zalcman, G. et al. Bevacizumab for newly diagnosed pleural mesothelioma in the Mesothelioma Avastin Cisplatin Pemetrexed Study (MAPS): a randomised, controlled, open-label, phase 3 trial. Lancet 387, 1405-1414, doi:10.1016/S0140-6736(15)01238-6 (2016).
10 Alley, E. W. et al. Clinical safety and activity of pembrolizumab in patients with malignant pleural mesothelioma (KEYNOTE-028): preliminary results from a non-randomised, open-label, phase 1b trial. The Lancet. Oncology 18, 623-630, doi:10.1016/S1470-2045(17)30169-9 (2017).
11 Quispel-Janssen, J. et al. Programmed Death 1 Blockade With Nivolumab in Patients With Recurrent Malignant Pleural Mesothelioma. Journal of thoracic oncology: official publication of the International Association for the Study of Lung Cancer 13, 1569-1576, doi:10.1016/j.jtho.2018.05.038 (2018).
12 Okada, M. et al. Clinical Efficacy and Safety of Nivolumab: Results of a Multicenter, Open-label, Single-arm, Japanese Phase II study in Malignant Pleural Mesothelioma (MERIT).
Clinical cancer research: an official journal of the American Association for Cancer Research 25, 5485-5492, doi:10.1158/1078-0432.CCR-19-0103 (2019).
13 Fennell, D. A. et al. CONFIRM: a double-blind, placebo-controlled phase III clinical trial investigating the effect of nivolumab in patients with relapsed mesothelioma: study protocol for a randomised controlled trial. Trials 19, 233, doi:10.1186/s13063-018-2602-y (2018).
14 Gandhi, L. et al. Pembrolizumab plus Chemotherapy in Metastatic Non-Small-Cell Lung Cancer. The New England journal of medicine 378, 2078-2092, doi:10.1056/NEJMoa1801005 (2018).
15 Nowak, A. K. et al. Durvalumab with first-line chemotherapy in previously untreated malignant pleural mesothelioma (DREAM): a multicentre, single-arm, phase 2 trial with a safety run-in. The Lancet. Oncology 21, 1213-1223, doi:10.1016/S1470-2045(20)30462-9 (2020).
16 Baas, P. et al. First-line nivolumab plus ipilimumab in unresectable malignant pleural mesothelioma (CheckMate 743): a multicentre, randomised, open-label, phase 3 trial. Lancet 397, 375386, doi:10.1016/S0140-6736(20)32714-8 (2021).
17 Hmeljak, J. et al. Integrative Molecular Characterization of Malignant Pleural Mesothelioma. Cancer discovery 8, 1548-1565, doi:10.1158/2159-8290.CD-18-0804 (2018).
18 Bueno, R. et al. Comprehensive genomic analysis of malignant pleural mesothelioma identifies recurrent mutations, gene fusions and splicing alterations. Nature genetics 48, 407-416, doi:10.1038/ng.3520 (2016).
19 Mansfield, A. S. et al. Neoantigenic Potential of Complex Chromosomal Rearrangements in Mesothelioma. Journal of thoracic oncology: official publication of the International Association for the Study of Lung Cancer 14, 276-287, doi:10.1016/j.jtho.2018.10.001 (2019).
20 Panou, V. & Roe, O. D. Inherited Genetic Mutations and Polymorphisms in Malignant Mesothelioma: A Comprehensive Review. International journal of molecular sciences 21, doi:10.3390/ijms21124327 (2020).
21 Testa, J. R. et al. Germline BAP1 mutations predispose to malignant mesothelioma. Nature genetics 43, 1022-1025, doi:10.1038/ng.912 (2011).
22 Bononi, A. et al. Heterozygous germline BLM mutations increase susceptibility to asbestos and mesothelioma. Proceedings of the National Academy of Sciences of the United States of America, doi:10.1073/pnas.2019652117 (2020).
23 Oken, M. M. et al. Toxicity and response criteria of the Eastern Cooperative Oncology Group. American journal of clinical oncology 5, 649-655 (1982).
24 Tsao, A. S. et al. A practical guide of the Southwest Oncology Group to measure malignant pleural mesothelioma tumors by RECIST and modified RECIST criteria. Journal of thoracic oncology: official publication of the International Association for the Study of Lung Cancer 6, 598-601, doi:10.1097/JTO.0b013e318208c83d (2011).
25 Chowell, D. et al. Evolutionary divergence of HLA class I genotype impacts efficacy of cancer immunotherapy. Nature medicine 25, 1715-1720, doi:10.1038/s41591-019-0639-4 (2019).
26 Napolitano, A. et al. Minimal asbestos exposure in germline BAP1 heterozygous mice is associated with deregulated inflammatory response and increased risk of mesothelioma. Oncogene 35, 1996-2002, doi:10.1038/one.2015.243 (2016).
27 Wang, T. et al. An Empirical Approach Leveraging Tumorgrafts to Dissect the Tumor Microenvironment in Renal Cell Carcinoma Identifies Missing Link to Prognostic Inflammatory Factors. Cancer discovery 8, 1142-1155, doi:10.1158/2159-8290.CD-17-1246 (2018).
28 Hassan, R. et al. Inherited predisposition to malignant mesothelioma and overall survival following platinum chemotherapy. Proceedings of the National Academy of Sciences of the United States of America 116, 9008-9013, doi:10.1073/pnas. 1821510116 (2019).
29 Bott, M. et al. The nuclear deubiquitinase BAP1 is commonly inactivated by somatic mutations and 3p21.1 losses in malignant pleural mesothelioma. Nature genetics 43, 668-672, doi:10.1038/ng.855 (2011).
30 Robert, C. et al. Immunotherapy discontinuation—how, and when? Data from melanoma as a paradigm. Nature reviews. Clinical oncology 17, 707-715, doi:10.1038/s41571-020-0399-6 (2020).
31 Litchfield, K. et al. Meta-analysis of tumor- and T cell-intrinsic mechanisms of sensitization to checkpoint inhibition. Cell 184, 596-614 e514, doi:10.1016/j.cell.2021.01.002 (2021)
32 Venkatesan, S. et al. Perspective: APOBEC mutagenesis in drug resistance and immune escape in HIV and cancer evolution. Annals of oncology: official journal of the European Society for Medical Oncology 29, 563-572, doi:10.1093/annonc/mdy003 (2018).
33 Rosenthal, R. et al. Neoantigen-directed immune escape in lung cancer evolution. Nature 567, 479-485, doi:10.1038/s41586-019-1032-7 (2019).
34 Bianchi, A. B. et al. High frequency of inactivating mutations in the neurofibromatosis type 2 gene (NF2) in primary malignant mesotheliomas. Proceedings of the National Academy of Sciences of the United States of America 92, 10854-10858, doi:10.1073/pnas.92.24.10854 (1995).
35 Zhang, M. et al. Clonal architecture in mesothelioma is prognostic and shapes the tumour microenvironment. Nature communications 12, 1751, doi:10.1038/s41467-021-21798-w (2021).
36 Pan, D. et al. A major chromatin regulator determines resistance of tumor cells to T cell-mediated killing. Science 359, 770-775, doi:10.1126/science.aao1710 (2018).
37 Shen, J. et al. ARID1A deficiency promotes mutability and potentiates therapeutic antitumor immunity unleashed by immune checkpoint blockade. Nature medicine 24, 556-562, doi:10.1038/s41591-018-0012-z (2018).
38 Nasu, M. et al. High Incidence of Somatic BAP1 alterations in sporadic malignant mesothelioma. Journal of thoracic oncology: official publication of the International Association for the Study of Lung Cancer 10, 565-576, doi:10.1097/JTO.0000000000000471 (2015).
39 Baumann, F. et al. Mesothelioma patients with germline BAP1 mutations have 7-fold improved long-term survival. Carcinogenesis 36, 76-81, doi:10.1093/carcin/bgu227 (2015).
40 Carbone, M. et al. Biological Mechanisms and Clinical Significance of BAP1 Mutations in Human Cancer. Cancer discovery 10, 1103-1120, doi:10.1158/2159-8290.CD-19-1220 (2020).
41 Samstein, R. M. et al. Mutations in BRCA1 and BRCA2 differentially affect the tumor microenvironment and response to checkpoint blockade immunotherapy. Nature cancer 1, 1188-1203, doi:10.1038/s43018-020-00139-8 (2021).
42 Ishikawa, H. & Barber, G. N. STING is an endoplasmic reticulum adaptor that facilitates innate immune signalling. Nature 455, 674-678, doi:10.1038/nature07317 (2008).
43 Anagnostou, V. et al. Evolution of Neoantigen Landscape during Immune Checkpoint Blockade in Non-Small Cell Lung Cancer. Cancer discovery 7, 264-276, doi:10.1158/2159-8290.CD-16-0828 (2017).
44 Anagnostou, V. et al. Integrative tumor and immune cell mutli-omic analyses to predict melanoma response to immune checkpoint blockade. Cell Reports Medicine (2020).
45 Binnewies, M. et al. Understanding the tumor immune microenvironment (TIME) for effective therapy. Nature medicine 24, 541-550, doi:10.1038/s41591-018-0014-x (2018).
46 Joshi, K. et al. Spatial heterogeneity of the T cell receptor repertoire reflects the mutational landscape in lung cancer. Nature medicine 25, 1549-1559, doi:10.1038/s41591-019-0592-2 (2019).
47 Thorsson, V. et al. The Immune Landscape of Cancer. Immunity 48, 812-830 e814, doi:10.1016/j.immuni.2018.03.023 (2018)
48 Ellrott, K. et al. Scalable Open Science Approach for Mutation Calling of Tumor Exomes Using Multiple Genomic Pipelines. Cell systems 6, 271-281 e277, doi:10.1016/j.cels.2018.03.002 (2018).
49 Jones, S. et al. Personalized genomic analyses for cancer mutation discovery and interpretation. Science translational medicine 7, 283ra253, doi:10.1126/scitranslmed.aaa7161 (2015).
50 Anagnostou, V. et al. Multimodal genomic features predict outcome of immune checkpoint blockade in non-small-cell lung cancer. Nature cancer 1, 99-111, doi:10.1038/s43018-019-0008-8 (2020).

### METHODS REFERENCES

51 Shao, X. M. et al. High-Throughput Prediction of MHC Class I and II Neoantigens with MHCnuggets. Cancer immunology research 8, 396-408, doi:10.1158/2326-6066.CIR-19-0464 (2020)
52 Kim, S. et al. Strelka2: fast and accurate calling of germline and somatic variants. Nature methods 15, 591-594, doi:10.1038/s41592-018-0051-x (2018).
53 Pagel, K. A. et al. Integrated Informatics Analysis of Cancer-Related Variants. JCO clinical cancer informatics 4, 310-317, doi:10.1200/CCI.19.00132 (2020).
54 Alexandrov, L. B. et al. Signatures of mutational processes in human cancer. Nature 500, 415-421, doi:10.1038/nature12477 (2013).
55 https.//CRAN.R-project.org/package=deconstructSigs.
56 Szolek, A. et al. OptiType: precision HLA typing from next-generation sequencing data.
Bioinformatics 30, 3310-3316, doi:10.1093/bioinformatics/btu548 (2014).
57 Xie, C. et al. Fast and accurate HLA typing from short-read next-generation sequence data with xHLA. Proceedings of the National Academy of Sciences of the United States of America 114, 8059-8064, doi:10.1073/pnas. 1707945114 (2017).
58 Cao, H. et al. An integrated tool to study MHC region: accurate SNV detection and HLA genes typing in human MHC region using targeted high-throughput sequencing. PloS one 8, e69388, doi:10.1371/journal.pone.0069388 (2013).
59 Shukla, S. A. et al. Comprehensive analysis of cancer-associated somatic mutations in class I HLA genes. Nature biotechnology 33, 1152-1158, doi:10.1038/nbt.3344 (2015).
60 McGranahan, N. et al. Allele-Specific HLA Loss and Immune Escape in Lung Cancer Evolution. Cell 171, 1259-1271 e1211, doi:10.1016/j.cell.2017.10.001 (2017).
61 Robinson, J. et al. The IPD and IMGT/HLA database: allele variant databases. Nucleic acids research 43, D423-431, doi:10.1093/nar/gku1161 (2015).
62 Shen, R. & Seshan, V. E. FACETS: allele-specific copy number and clonal heterogeneity analysis tool for high-throughput DNA sequencing. Nucleic acids research 44, e131, doi:10.1093/nar/gkw520 (2016).
63 Niknafs, N., Beleva-Guthrie, V., Naiman, D. Q. & Karchin, R. SubClonal Hierarchy Inference from Somatic Mutations: Automatic Reconstruction of Cancer Evolutionary Trees from Multi-region Next Generation Sequencing. PLOS computational biology 11, e1004416, doi:10.1371/journal.pcbi.1004416 (2015).
64 Sztupinszki, Z. et al. Migrating the SNP array-based homologous recombination deficiency measures to next generation sequencing data of breast cancer. NPJ breast cancer 4, 16, doi:10.1038/s41523-018-0066-6 (2018).
65 Carlson, C. S. et al. Using synthetic templates to design an unbiased multiplex PCR assay. Nature communications 4, 2680, doi:10.1038/ncomms3680 (2013).
66 Dobin, A. & Gingeras, T. R. Mapping RNA-seq Reads with STAR. Current protocols in bioinformatics 51, 11 14 11-19, doi:10.1002/0471250953.bi1114s51 (2015).
67 Li, B. & Dewey, C. N. RSEM: accurate transcript quantification from RNA-Seq data with or without a reference genome. BMC bioinformatics 12, 323, doi:10.1186/1471-2105-12-323 (2011).
68 Love, M. I., Huber, W. & Anders, S. Moderated estimation of fold change and dispersion for RNA-seq data with DESeq2. Genome biology 15, 550, doi:10.1186/s13059-014-0550-8 (2014).
69 Danilova, L. et al. The Mutation-Associated Neoantigen Functional Expansion of Specific T Cells (MANAFEST) Assay: A Sensitive Platform for Monitoring Antitumor Immunity. Cancer immunology research 6, 888-899, doi:10.1158/2326-6066.CIR-18-0129 (2018).
70 Zhang, J., Ji, Z. & Smith, K. N. Analysis of TCR beta CDR3 sequencing data for tracking anti-tumor immunity. Methods in enzymology 629, 443-464, doi:10.1016/bs.mie.2019.08.006 (2019).

### Example 3

The current working hypothesis for tumor-intrinsic features that determine the magnitude of anti-tumor immune responses relies on the assumption that each mutation contributes equally to a composite measure of tumor foreignness, reflected in the number of sequence alterations per coding DNA sequence or tumor mutation burden (TMB). However, with the exception of mismatch repair deficient tumors, TMB has failed to consistently demonstrate clinical utility in predicting responses to cancer immunotherapy. Efforts to separate subsets of alterations that may predominantly drive an effective anti-tumor immune response have yet to reveal a universal genomic predictive biomarker^{1,2}.

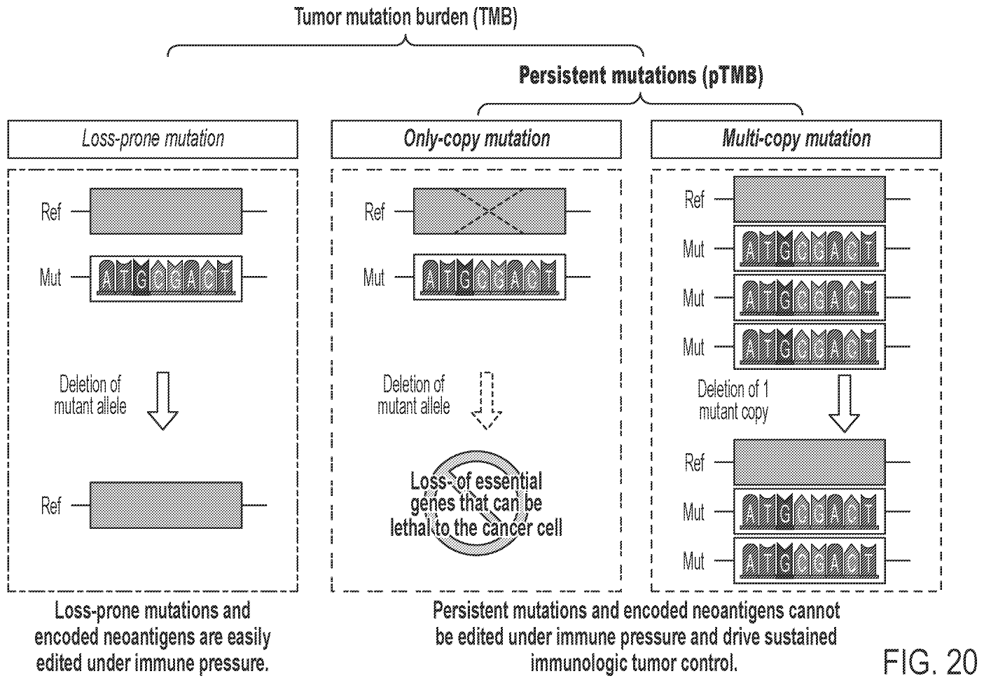

It was hypothesized, herein, that tumors with a higher frequency of sequence alterations in either haploid regions or in multiple copies would have a fitness disadvantage in the context of immunotherapy, as these alterations would continuously render them visible to the immune system, resulting in sustained tumor immunologic tumor control. Deletions of single copy alleles through chromosomal loss are typically not tolerated in cancer cells unless they are relatively small homozygous deletions⁵, as larger chromosomal deletions would contain essential genes in linkage with the mutation and would be lethal. Similarly, mutation loss by chromosomal deletions and loss of heterozygosity³ is evolutionary unlikely when mutations are contained in multiple copies. Therefore, these “persistent” mutations (which we hereafter refer to as pTMB) may function as an intrinsic driver of tumor rejection in the tumor microenvironment (FIG. 20).

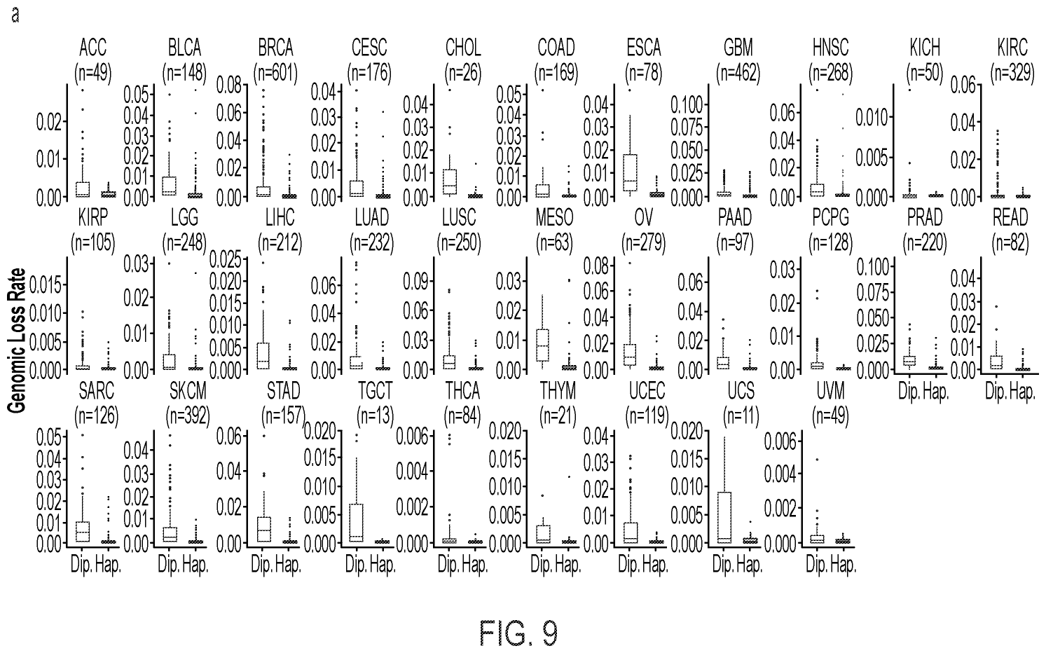

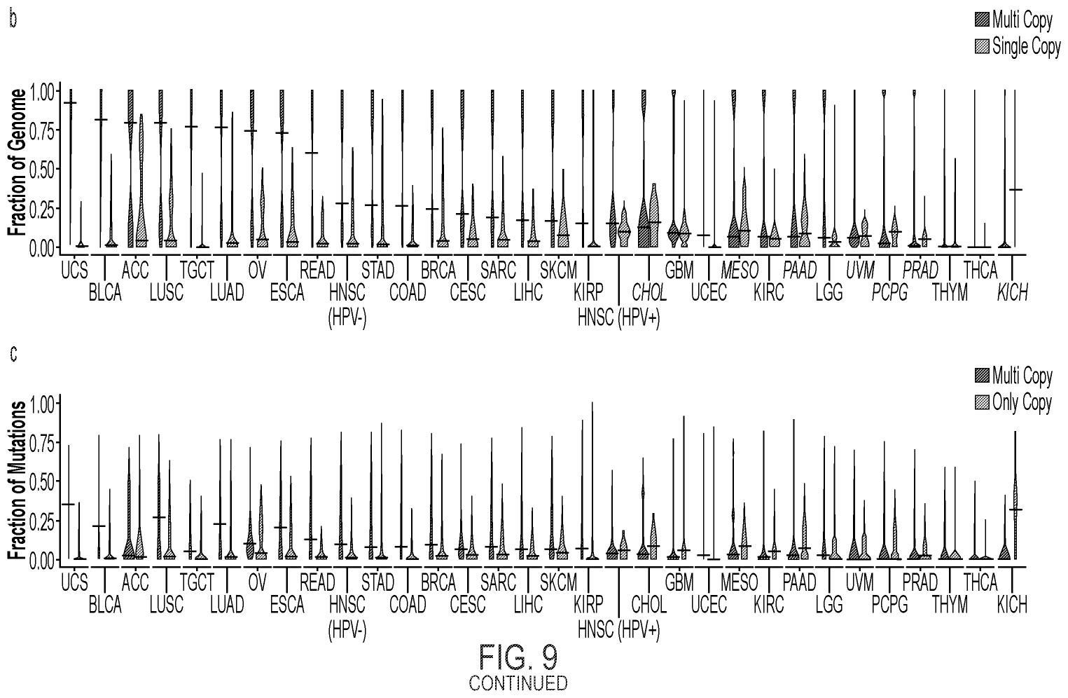

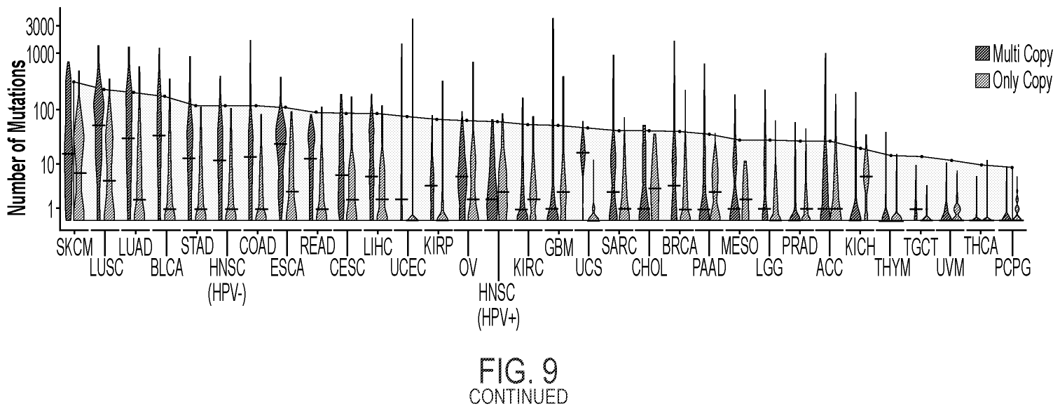

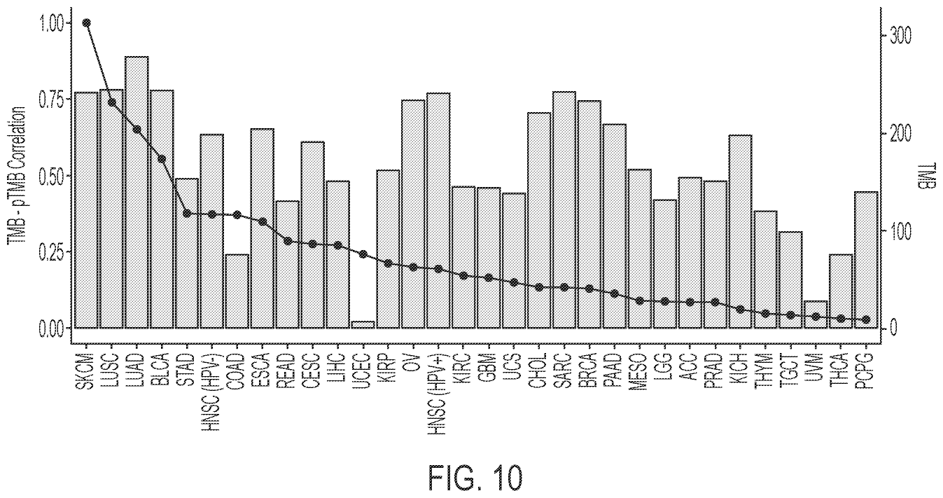

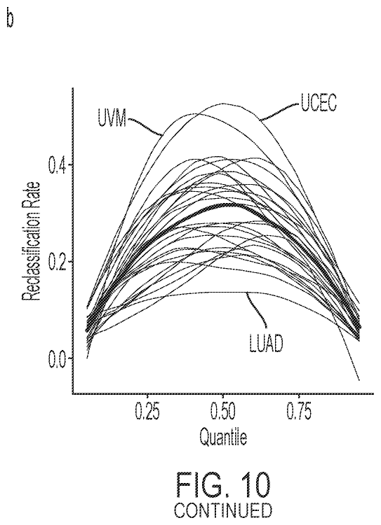

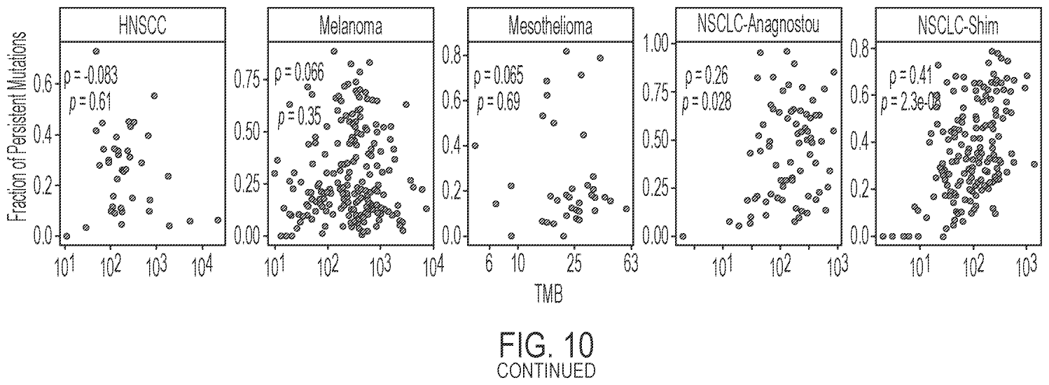

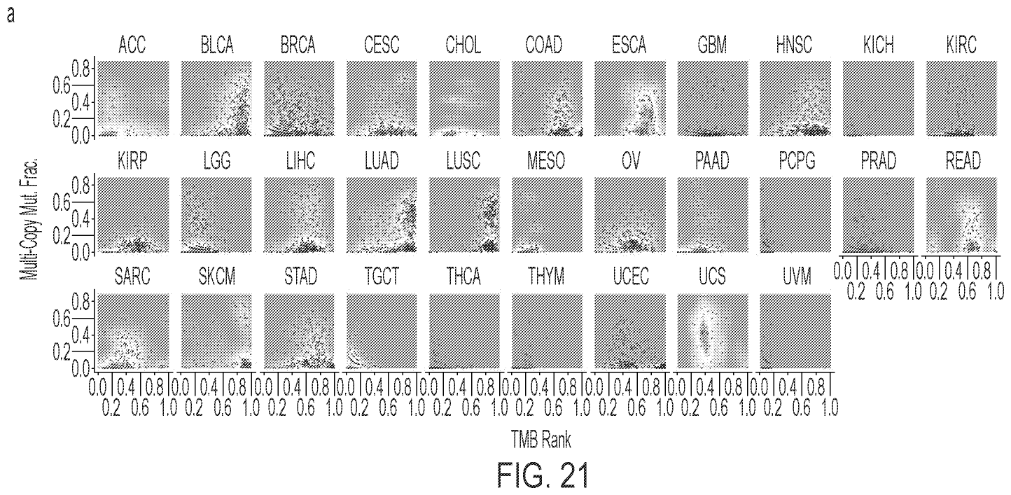

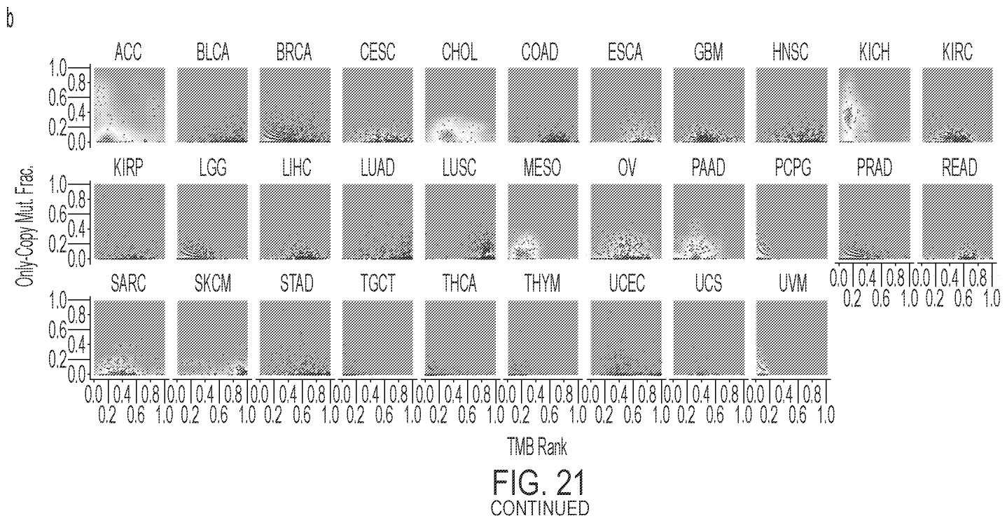

To investigate these hypotheses in a pan-cancer manner, we first evaluated the rate of loss in regions of the genome with a single copy per cell (haploid) versus euploid regions (2 copies per cell) using copy number profiles of 5,244 tumors across 31 tumor types from The Cancer Genome Atlas (TCGA), including immunotherapy responsive cancers such as melanoma, non-small cell lung cancer (NSCLC) and mesothelioma. These analyses revealed that the rate of loss in haploid regions was consistently lower than that in euploid regions (FIG. 9A), supporting the notion that mutations contained in these regions would be difficult to eliminate. We then examined the frequency of haploid and polyploid regions across the genome and quantified the fraction of the genome in single-copy vs multi-copy states (N=9,991; FIG. 9B. Some tumor types, including endometrial carcinosarcomas, bladder cancers, adrenocortical carcinomas, lung squamous carcinomas, lung adenocarcinomas, ovarian cancers and cutaneous melanomas were enriched for genomic regions in the multi-copy state, while cholangiocarcinomas, pancreatic adenocarcinomas, mesotheliomas and kidney chromophobe tumors showed a higher genome fraction in the single-copy state (FIG. 9B). Integration of sequence alterations in only-copy and multi-copy states for these cancers revealed a cancer lineage-dependent distribution of persistent mutations across 9,242 tumors (FIG. 9B C-9D). Next, we characterized the distribution of persistent mutation load in the background of the overall TMB within each tumor type, and found that TMB does not fully explain the abundance of multi-copy and only-copy mutations, as tumor types with similar TMB exhibited differences in multi-copy and only-copy mutation content (N=9,242; FIG. 9D). Notably, a wide range of prevalence of mutations in only-copy or multi-copy states was observed across the entire range of overall tumor mutation burden, suggesting that persistent mutations provide a measure of alterations that is distinct from TMB (FIG. 21). We further evaluated the degree of correlation between TMB and pTMB and found a significant degree of variation in their association across the 31 tumor types analyzed (Spearman p: median 0.49, range 0.02-0.89; FIG. 10A). Similar patterns were observed when multi-copy (Spearman p median: 0.42, range: 0.02-0.76) and single-copy mutations (Spearman p median: 0.21, range: −0.12-0.48) were considered separately. To understand the potential re-classification of tumors based on pTMB, we employed a series of quantile values ranging from 5% to 95% to define high/low groups for TMB and pTMB (Methods) These analyses revealed re-classification rates as high as 53% in individual tumor types (range 15%-53%), with a median reclassification rate of 33% across all tumor types (FIG. 10B and FIG. 22).

Next, we explored the relationship between persistent mutation content and TMB in seven published ICB cohorts across three tumor types (n=485; melanoma⁶⁻⁸, NSCLC^{2,9} and mesothelioma¹⁰) and a new cohort of patients with HPV negative (HPV−) head and neck cancer (HNSCC) who received ICB (n=39). Similar to the TCGA analyses, we did not detect a significant enrichment for a higher persistent mutation fraction in tumors harboring a higher TMB in the HNSCC (Spearman ρ=−0.083, p=0.61), melanoma (Spearman ρ=0.066, p=0.35) and mesothelioma cohorts (Spearman ρ=0.065, p=0.69), while a weak correlation between TMB and persistent mutation fraction was observed in the NSCLC cohorts (NSCLC-Anagnostou: Spearman ρ=0.26, p=0.03, NSCLC-Shim: Spearman ρ=0.41, p=2.3e-08; FIG. 10C). Collectively, these findings further support the notion that pTMB prevalence is distinct from TMB and that tumors are differentially ranked by their pTMB or TMB content in a cancer lineage-dependent manner.

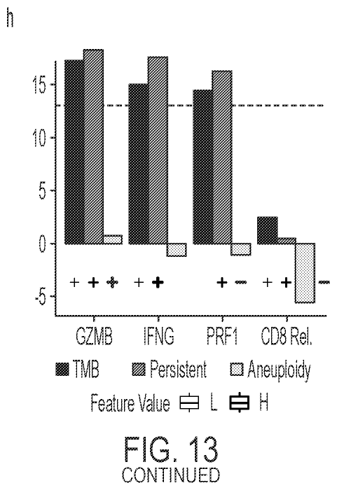

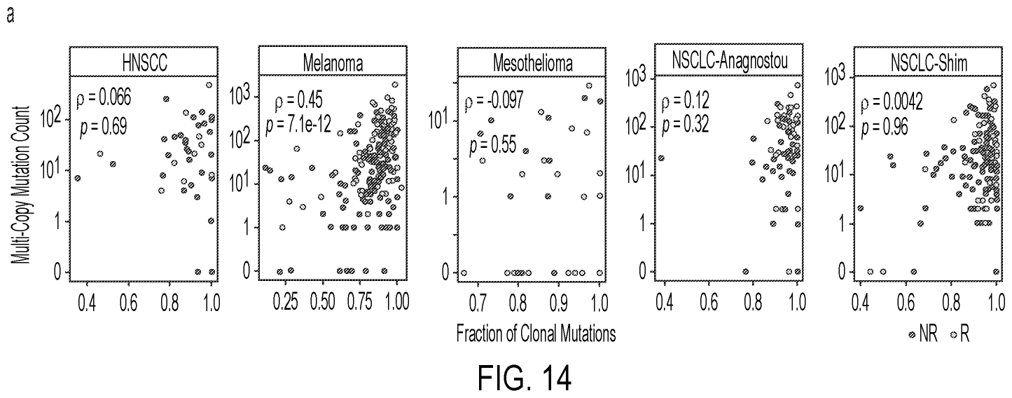

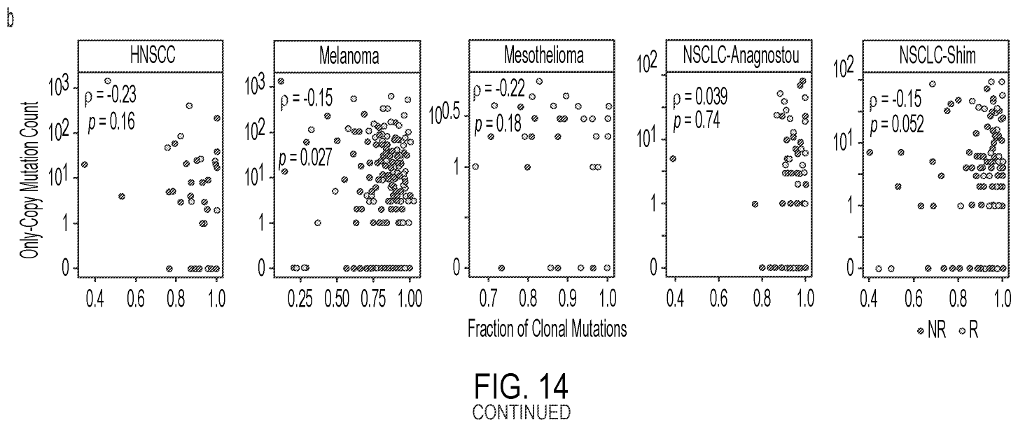

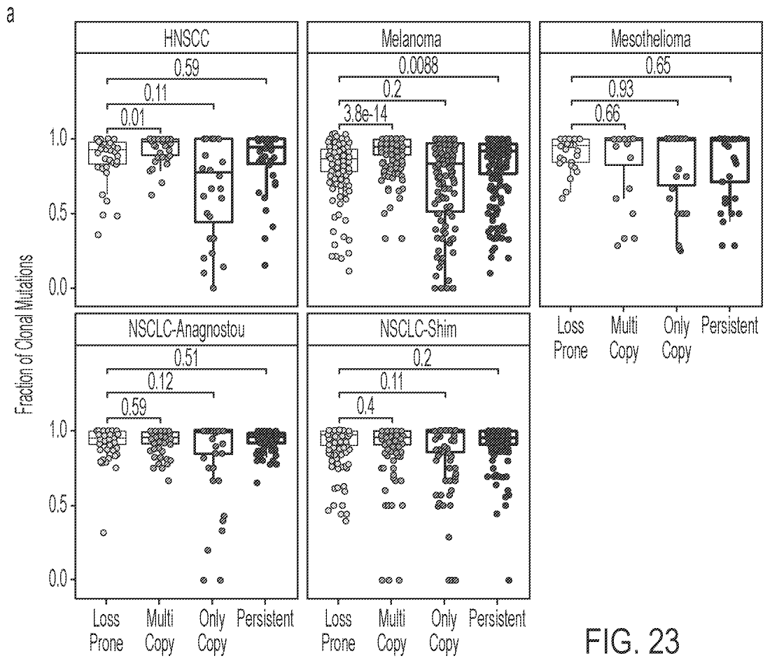

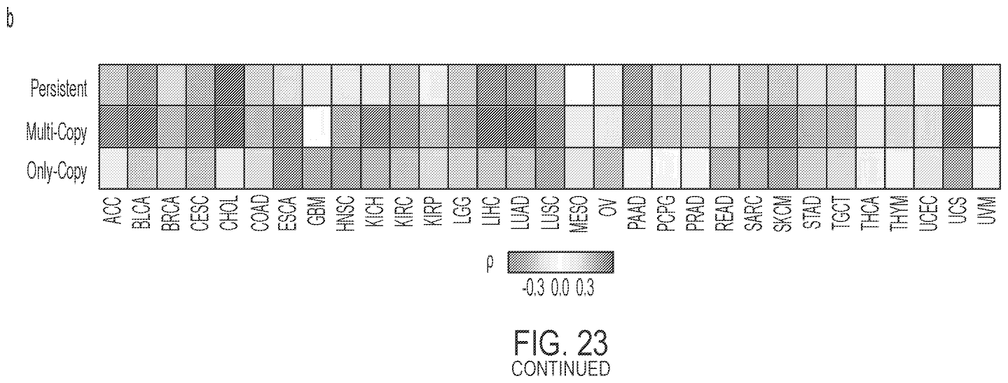

We theorized that the impact of pTMB would be exemplified in the context of treatment with immune checkpoint blockade (ICB), where inherent anti-tumor immune responses would be enhanced and sustained in the presence of continued persistent mutation-associated neoantigen (pMANA) stimulation. As our analyses pointed towards subsets of mutations within TMB that may carry differential weights in demarcating tumor foreignness, we evaluated persistent mutations in comparison to mutations that are more likely to be lost in the context of tumor evolution. We refer to the latter as “loss-prone” mutations and these account for the majority of coding alterations that constitute a tumor's TMB. We next asked the question whether there are differential clonal compositions between the persistent and loss-prone mutation subsets. In the HNSCC, mesothelioma, and NSCLC cohorts, we did not detect a difference in the fraction of clonal alterations between persistent mutations and loss-prone mutations, while in the melanoma cohort, persistent mutations tended to be more clonal (FIG. 23). When multi-copy mutations were considered separately, higher cellular fractions were noted for the multi-copy subset compared to loss-prone mutations in the melanoma and HNSCC cohorts, in line with the notion that multi-copy mutations may be acquired prior to somatic copy number gains and are thus enriched for more clonal events. To further study the clonal architecture of persistent mutations, we evaluated the correlation between pTMB and the fraction of clonal mutations in 31 TCGA tumor types and in the ICB cohorts. In the TCGA dataset, we observed a wide range of correlations between pTMB and fraction of clonal mutations (Spearman ρ range: −0.11-0.59; FIG. 23). We found a moderate degree of anti-correlation between clonal mutation fraction and the number of only-copy mutations in most tumor types (Spearman ρ range: −0.57-0.07), while a moderate degree of correlation was detected between the fraction of clonal mutations and multi-copy mutations (Spearman ρ range: 0.01-0.60, FIG. 23). In the ICB cohorts, we did not detect strong correlations between tumor clonal heterogeneity and persistent mutations (Spearman ρ range: −0.25-0.33) or within the multi-copy and only-copy subsets (FIG. 14). These findings suggest that persistent mutations are seen at the full spectrum of tumor clonal heterogeneity.

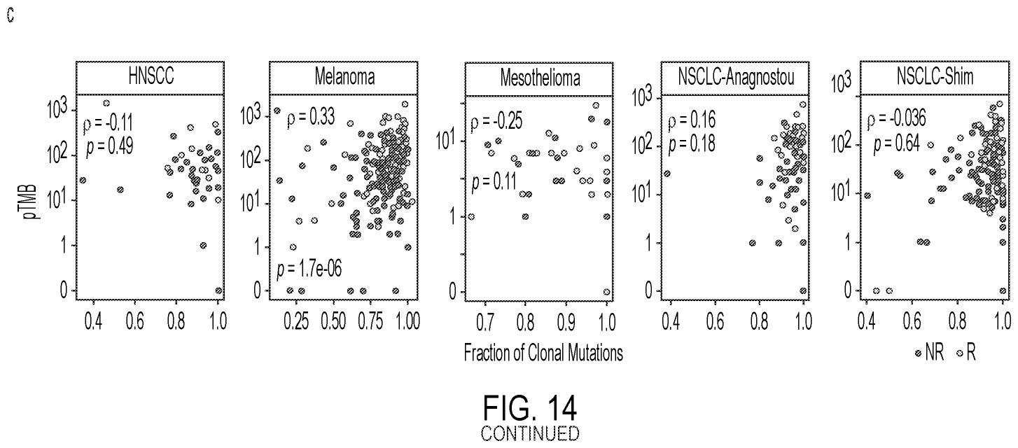

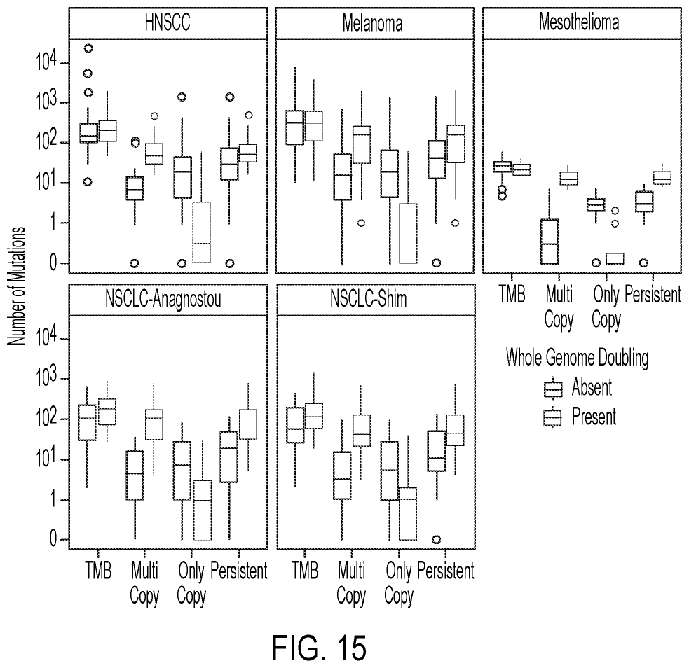

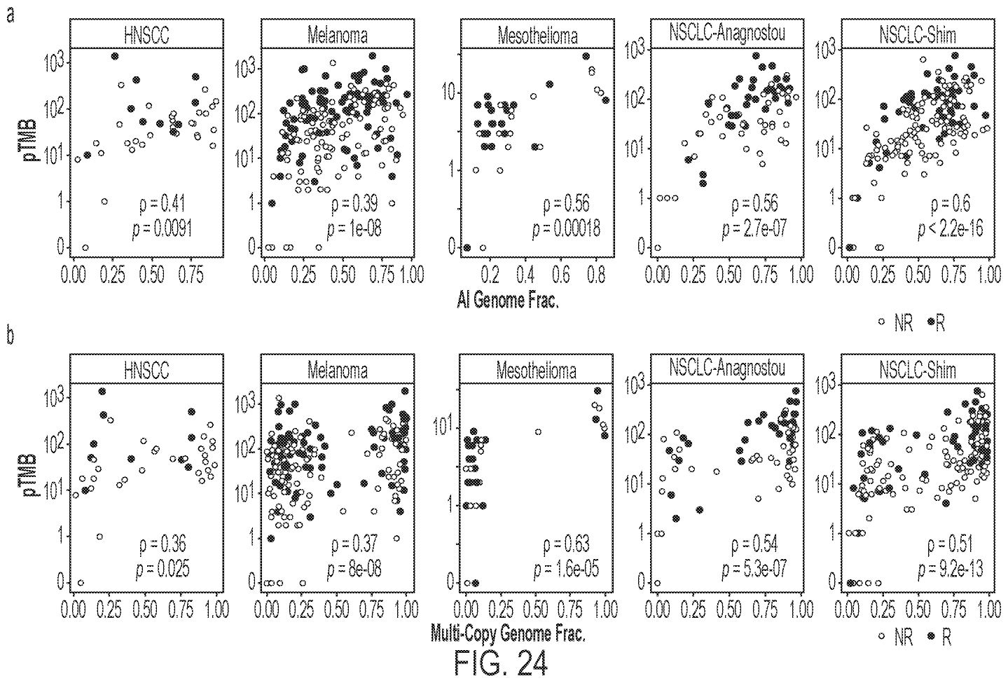

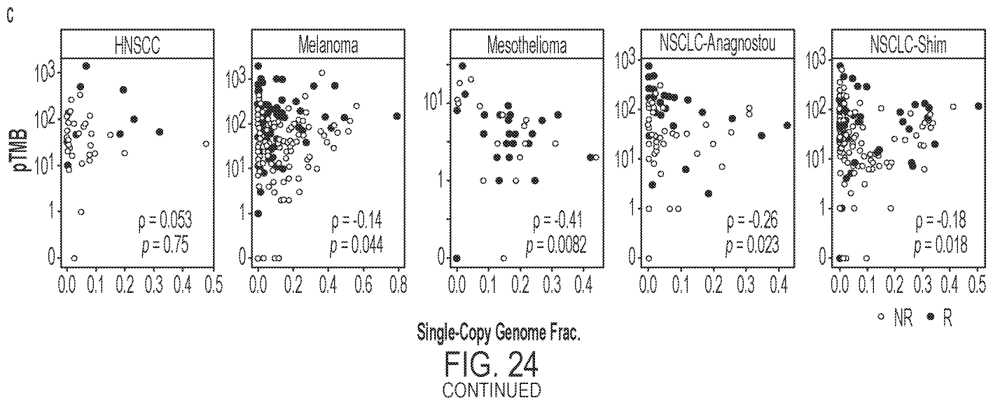

Conceptually, persistent mutations—that by definition reside in aneuploid regions of the genome—are integrally linked with tumor aneuploidy and next, we assessed the relationship between persistent mutations and fraction of the genome with allelic imbalance. In the ICB cohorts, a moderate degree of correlation was observed between the extent of tumor aneuploidy and pTMB (Spearman ρ range: 0.39-0.60; FIG. 24). In parallel, we evaluated tumors for whole-genome doubling events, which would enable the acquisition of additional mutant copies of the mutations present before the doubling event, and may therefore be a significant contributor to pTMB. Indeed, in all ICB cohorts analyzed, tumors with whole genome doubling also harbored a higher number of multi-copy mutations (HNSCC; Mann Whitney p=1.6e-05, melanoma; p=3.14e-14, mesothelioma; p=8.24e-06, NSCLC-Anagnostou; p=6.82e-09, NSCLC-Shim; p=9.3e-17; FIG. 15). Notably, tumors which have undergone whole genome doubling are by definition expected to have a very small fraction of the genome at total copy number of 1, and consistent with this notion, we observed a much lower prevalence of only-copy mutations in genomes with whole genome doubling events.

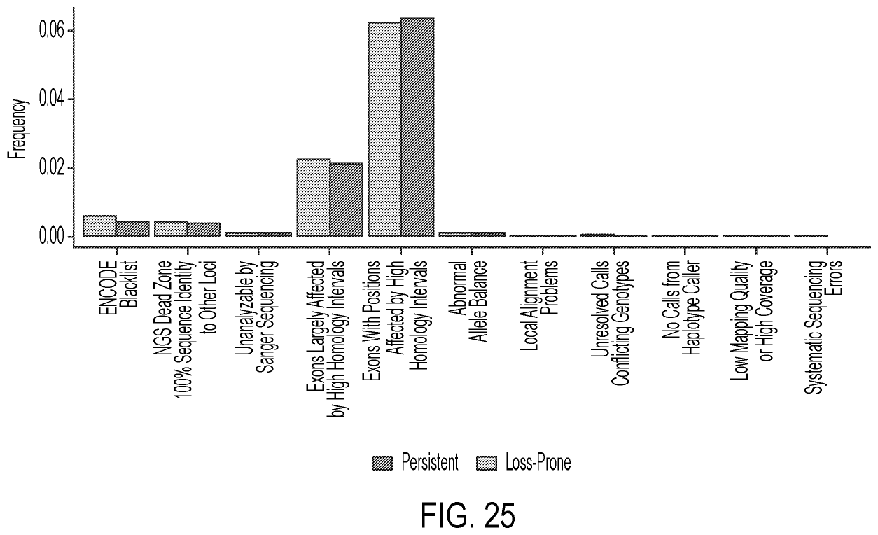

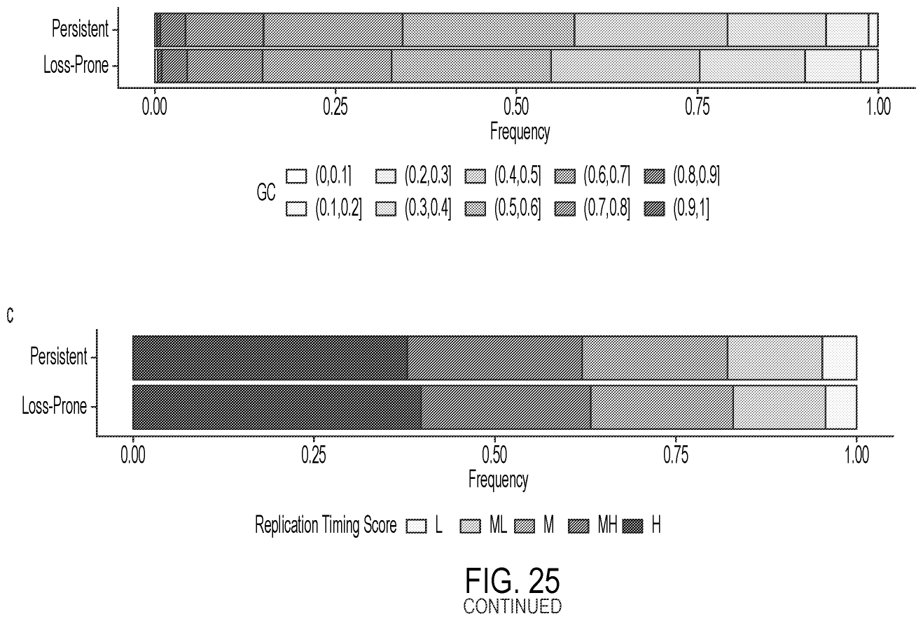

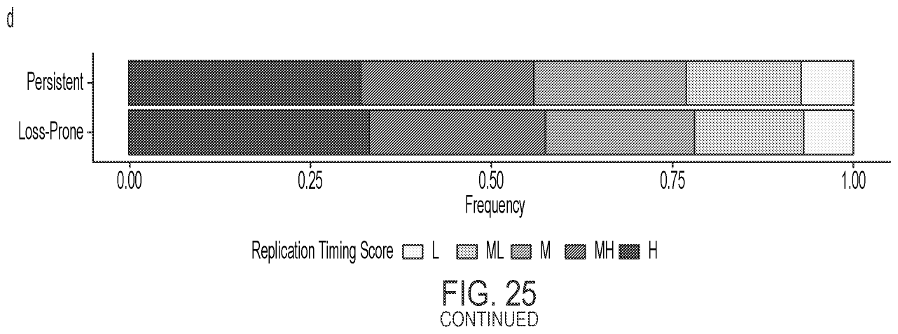

We investigated potential bias related to different timing of acquisition of persistent mutations, background mutation rates and accuracy of mutation calls in these loci and evaluated the distribution of sequence properties such as GC content and replication timing as well as mutation call quality in persistent versus loss-prone mutations utilizing exome data from 9,242 tumors from TCGA. (FIG. 25). We found a similar GC composition surrounding loci with persistent and loss-prone mutations (Cohen's d=0.08, persistent mean=0.54, loss-prone mean=0.52) across cancers. We then performed a cancer lineage-specific evaluation of replication timing in the melanoma and NSCLC subsets and found a similar distribution in persistent and loss-prone mutations (SKCM Cohen's d=−0.035, NSCLC Cohen's d=−0.032). Furthermore, we quantified the fraction of mutations in each category that could theoretically be affected by limitations of NGS analysis^{11,12} and found similar distributions in persistent and loss-prone mutations (FIG. 25). These findings suggest that persistent mutation calling is not confounded by background mutation rates, replication timing or technical artifacts

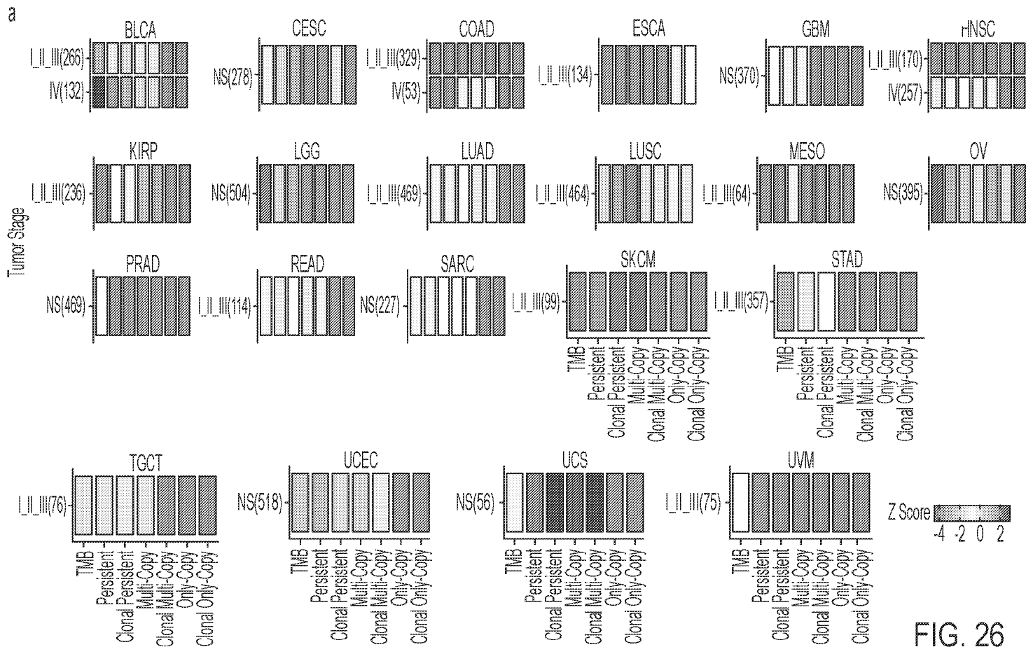

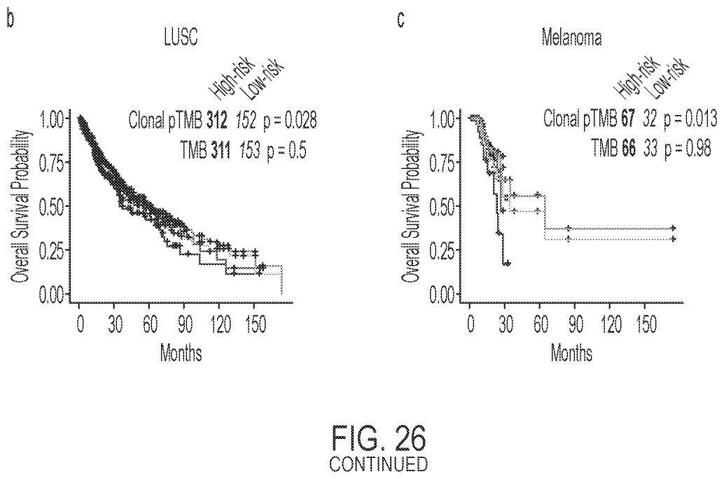

We then evaluated whether a higher pTMB was linked with clinical outcome in patients with previously untreated tumors from the TCGA (Methods). Our analyses showed that the association between persistent mutation load and clinical outcome was context-dependent; whereby a significant association with prolonged overall survival was noted for lung squamous cell carcinoma (pTMB: 56.27 vs 43.86 months, log-rank p=0.085; clonal pTMB: 60.48 vs 35.32 months, log-rank p=0.028), melanoma (pTMB: 65.83 vs 23.69 months, log-rank p=0.036; clonal pTMB: 65.83 vs 23.69 months, log-rank p=0.013), and uterine carcinosarcoma (pTMB: 27.53 vs 17.15 months, log-rank p=0.021; clonal pTMB: 50.13 vs 14.68 months, log-rank p=2.66e-03) but not for any other cancer type studied (FIG. 26). Tumor mutation burden was more weakly associated with overall survival in the lung squamous cell carcinoma (log-rank p=0.50), melanoma (log-rank p=0.98), and uterine carcinosarcoma (log-rank p=0.17) sets.

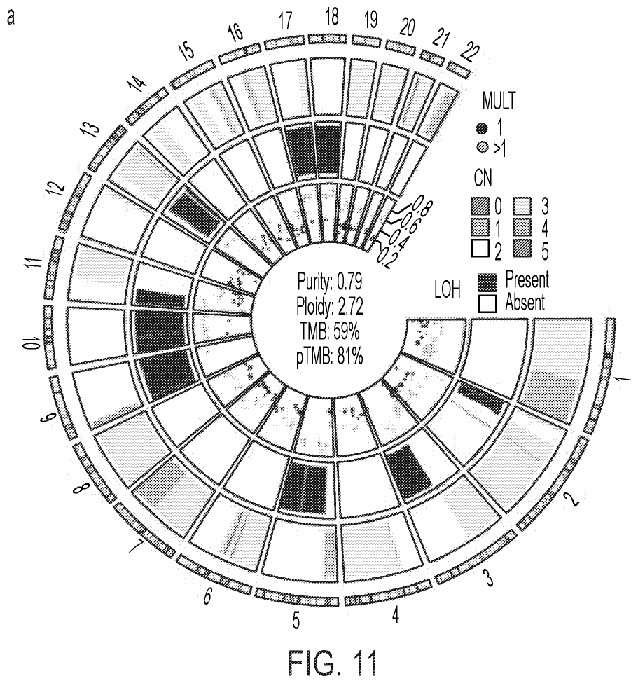

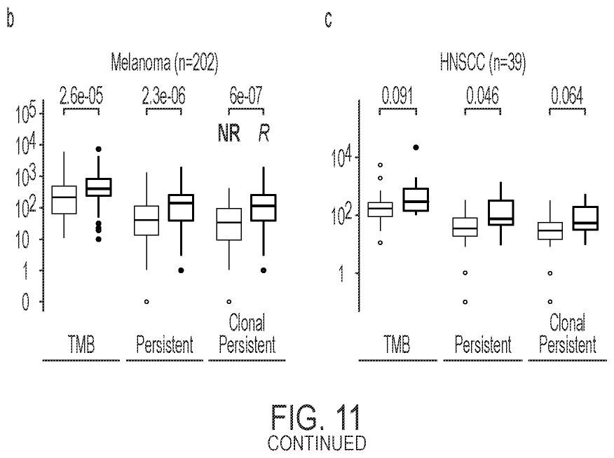

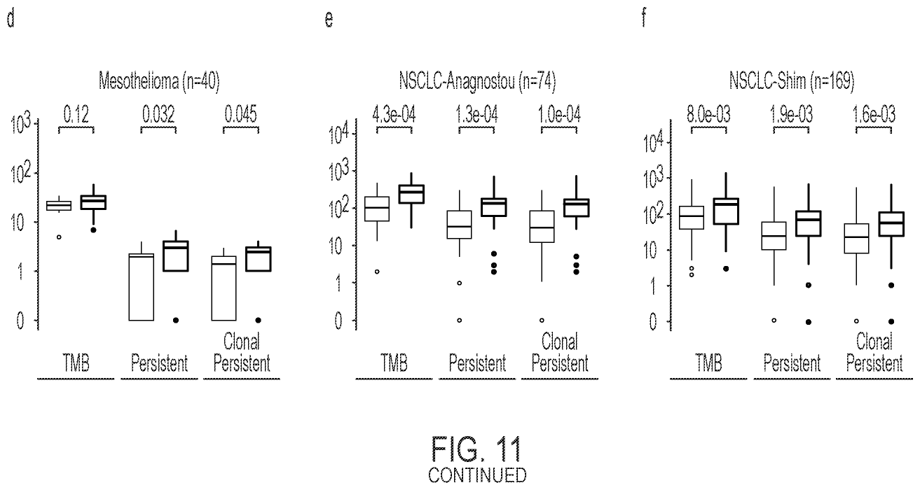

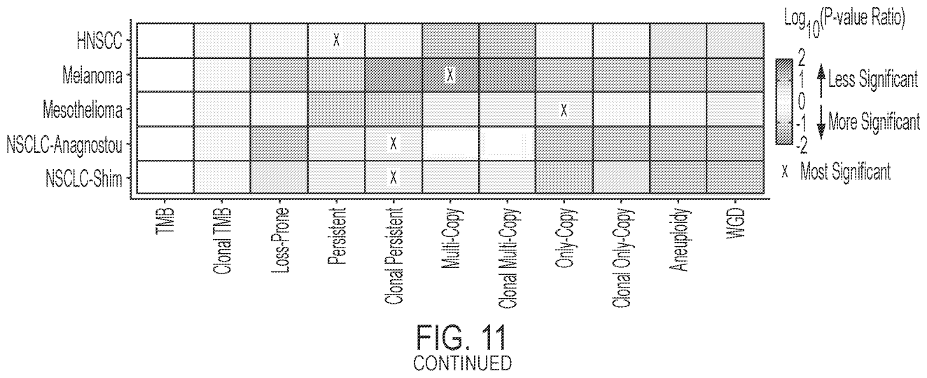

Importantly, we hypothesized that tumors with a high persistent mutation content would be the most visible” to the immune system and would therefore regress in the context of immunotherapy, a phenomenon that would be reflected in sustained clinical responses to therapy. To this end, we evaluated the potential of pTMB, multi-copy and only-copy mutations in predicting response to immune checkpoint blockade in 542 patients with melanoma, NSCLC, mesothelioma and HNSCC (Methods). We discovered that tumors with a high pTMB attained higher rates of therapeutic response with ICB, while TMB alone or the number of loss-prone mutations less optimally distinguished responding from non-responding tumors (FIG. 11). As a representative example that illustrates the difference between pTMB and TMB, patient Pt44 with metastatic melanoma harboring a pTMB in the 81% quantile but in the 59% quantile by TMB attained a prolonged progression-free survival (18.4 months) on ICB (FIG. 11A). High pTMB, more accurately differentiated responding from non-responding tumors in the melanoma cohort (n=202, Mann Whitney U-test p=2.3e-06, p-6.0e-07, p=1.92e-03 and p=2.6e-05 for pTMB, clonal pTMB, loss-prone mutation load, and TMB respectively; FIG. 11B). Similarly, in the HNSCC ICB cohort pTMB was associated with therapeutic response (n=39, Mann Whitney U-test p=0.05, p=0.06, p=0.16 and p=0.09 for pTMB, clonal pTMB, loss-prone mutations, and TMB respectively; FIG. 11C). In patients with unresectable pleural mesotheliomas, pTMB outperformed TMB in predicting response to durvalumab plus platinum-pemetrexed chemotherapy (n=40, Mann Whitney U-test p=0.03, p=0.05, p=0.09, and p=0.12 for pTMB, clonal pTMB, loss-prone mutations and TMB respectively, FIG. 11D). A higher pTMB differentiated responding from non-responding non-small cell lung cancers (NSCLC-Anagnostou, n=74, Mann Whitney U-test p=1.3e-04, p=1.0e-04, p=0.01, and p=4.3e-04 for pTMB, clonal pTMB, loss-prone mutations, and TMB respectively; NSCLC-Shim, n=169, p=1.9e-03, p=1.6e-03, p=0.03, and p=8.0e-03 for pTMB, clonal pTMB, loss-prone mutations, and TMB respectively; FIG. 11E-11F).

Next, we evaluated the effect size of persistent mutations, loss-prone mutations, and TMB on clinical outcome. In the melanoma, HNSCC and mesothelioma cohorts, the effect size for pTMB was larger than TMB or loss-prone mutations (HNSCC: pTMB Cohen's d=−0.96, TMB d=−0.64, loss-prone d=−0.61; melanoma: pTMB d=−0.57, TMB d=−0.44, loss-prone d=−0.35; mesothelioma: pTMB d=−0.74, TMB d=−0.51, loss-prone d=−0.58), highlighting the importance of pTMB in informing therapeutic response to ICB. In the NSCLC cohorts, the effect size for pTMB, while very close to that of TMB, clearly exceeded the effect size of loss-prone mutations (NSCLC-Anagnostou: pTMB d=−0.89, TMB d=−0.93, loss-prone d=−0.58; NSCLC-Shim: pTMB d=−0.53, TMB d=−0.54, loss-prone d=−0.44). These findings suggest that the power of TMB to distinguish between responding and non-responding tumors in the context of ICB is largely driven by the persistent mutation content. Notably, in the NSCLC cohort, clonal pTMB more optimally distinguished responding from non-responding tumors (Mann Whitney U-test p=1.03e-04 and p=1.60e-03 for NSCLC-Anagnostou and NSCLC-Shim respectively). In the melanoma cohort, the number of multi-copy mutations was tightly correlated with therapeutic response (Mann Whitney U-test p=5.42e-07), while in the mesothelioma cohort, the number of only-copy mutations better distinguished responding and non-responding tumors (Mann Whitney U-test p=3.15e-02; FIG. 11G). Importantly, pTMB outperformed loss-prone mutation content in all ICB cohorts, despite the latter representing a larger fraction of the tumor mutation burden (Mann Whitney U-test p=1.92e-03 vs 2.25e-06, p=0.16 vs 0.05, p=0.09 vs 0.03, 1.03e-02 vs 1.26e-04, p=3.20e-02 vs 1.87e-03 for loss-prone vs pTMB in melanoma, HNSCC, mesothelioma, NSCLC-Anagnostou, and NSCLC-Shim respectively; FIG. 11G). We next asked the question whether the tumors differentially classified by pTMB compared to TMB have different responses to ICB. To this end, we evaluated the number of cases with differential pTMB/TMB classification and compared the therapeutic response rates between pTMB-low/TMB-high and pTMB-high/TMB-low tumors. In the melanoma cohort, 23 tumors fell in the TMB-high/pTMB-low and 23 tumors fell in the TMB-low/pTMB-high category. We found a higher frequency of responding tumors in pTMB-high/TMB-low category compared to the pTMB-low/TMB-high group (Fisher's exact p=0.04, pTMB-high/TMB-low group: 16 responders, 7 non-responders; pTMB-low/TMB-high group: 8 responders, 15 non-responders). These findings tie into the reclassification analyses from the larger TCGA cohort, and together support the differential classification of tumors based on their persistent mutation content, that is reflective of improved outcomes in the ICB setting.

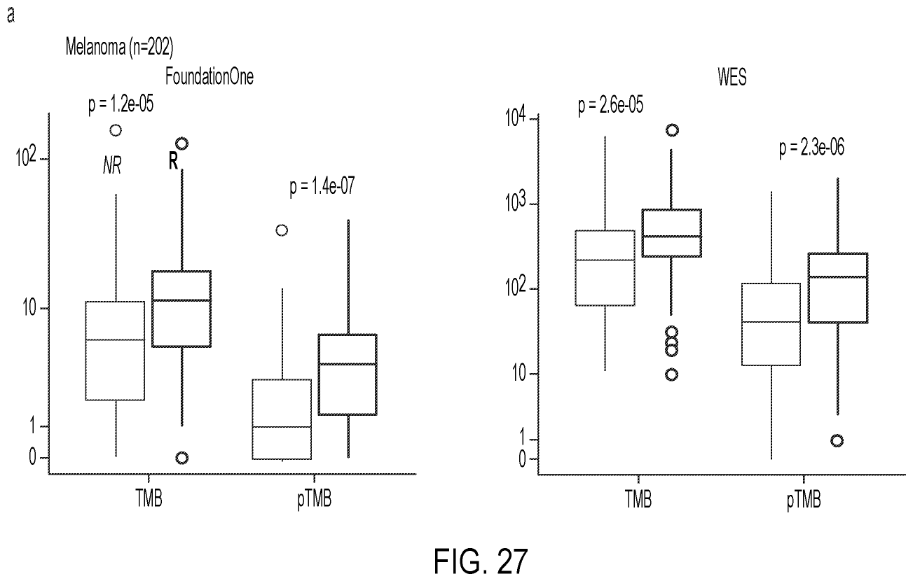

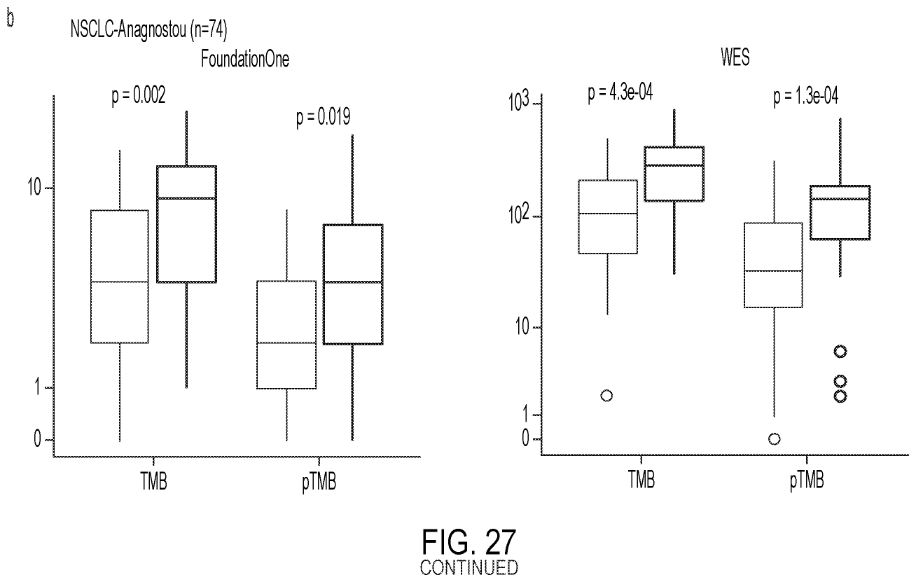

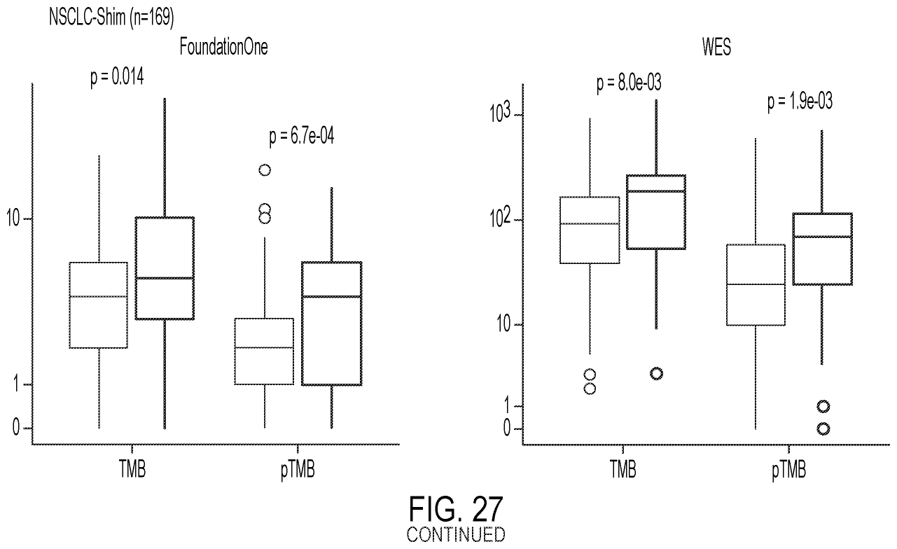

To further explore the immediate clinical utility of pTMB, we evaluated the feasibility of estimating pTMB from gene panel targeted next-generation sequencing, that is widely used in clinical cancer care. Using the genomic intervals from a widely-adopted clinical targeted NGS gene panel (309 genes; Methods), we performed in silico simulations utilizing whole exome sequence data from the melanoma and NSCLC ICB cohorts and computed TMB and pTMB in each tumor as captured by the region of interest of the targeted NGS panel. pTMB more accurately differentiated responding from non-responding tumors (melanoma, n=202, p=1.37E-07 for pTMB and p=1.22E-05 for TMB; NSCLC-Shim, n=169, p=6.7E-04 for pTMB and p=0.014 for TMB; NSCLC-Anagnostou, n=74, p=0.02 for pTMB and p=2.0E-03 for TMB; Mann Whitney U-test; FIG. 27).

We previously demonstrated the association between tumor aneuploidy and persistent mutations and as tumor aneuploidy has been associated with inferior outcomes to ICB, likely in the context of an immune excluded tumor microenvironment¹³, we investigated whether pTMB has an incremental value over tumor aneuploidy and whole genome doubling (WGD) events in predicting therapeutic response. Tumor aneuploidy (Mann Whitney U-test p=0.35, p=0.73, p=0.35, p=0.07, p=0.50 for the NSCLC-Anagnostou, NSCLC-Shim, melanoma, mesothelioma and HNSCC cohorts respectively) or occurrence of WGD alone (Fisher's exact p=0.43, p=0.73, p=0.11, p=0.23, p-0.48 for the NSCLC-Anagnostou, NSCLC-Shim, melanoma, mesothelioma and HNSCC cohorts respectively) failed to predict response to ICB in all cohorts assessed (FIG. 11G).

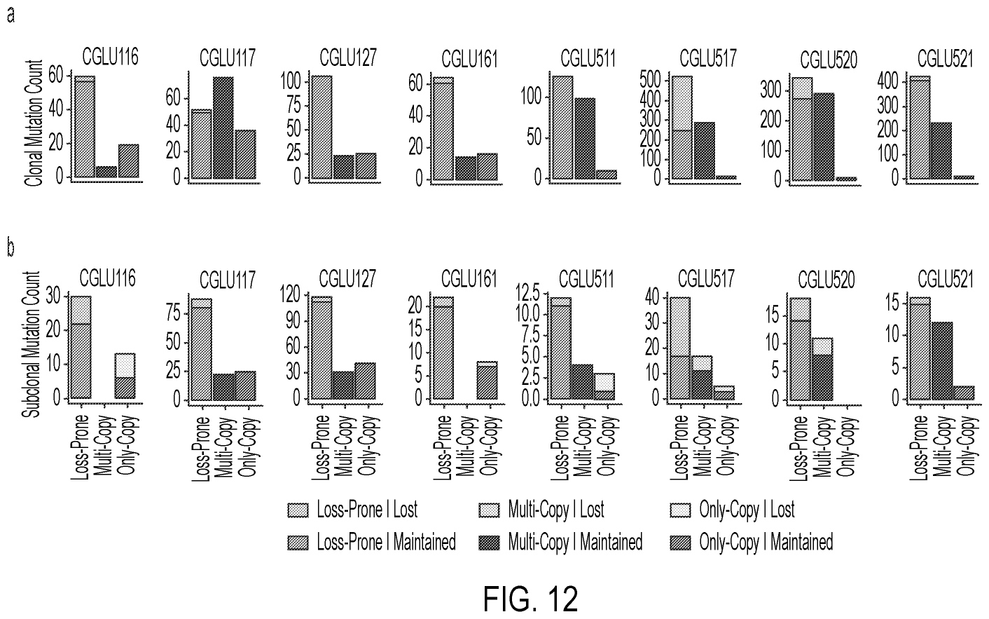

To establish the biological plausibility of persistent mutations in the context of tumor evolution, we performed serial whole exome sequencing analyses of longitudinal tumor samples before and after ICB treatment. We hypothesized that clonal persistent mutations would not be eliminated in the context of tumor evolution under the selective pressure of immunotherapy, as they are unlikely to undergo subclonal elimination in the context of therapy and also unlikely to be lost by chromosomal deletions (potentially lethal in the case of mutations residing in single copy regions and biologically implausible in the multiple copy regions). Consistent with our hypothesis, in analyzing pre-treatment and post-acquired resistance tumor samples from 8 patients with NSCLC treated with ICB (Methods), we discovered a marked difference in the frequency of loss between clonal persistent and loss-prone mutation sets. Across 16 serially biopsied tumors from 8 patients, a total of 363 out of 2836 clonal mutations that were detected in the baseline tumor were lost in the descendent tumor. Of these, the vast majority were clonal loss-prone mutations (358 out of 363, 98.6%). In 6 out of 8 patients analyzed, no clonal persistent mutation was lost in the descendent tumor, and of the two remaining patients, each had two clonal multi-copy mutations that were not detected in the descendent tumor, suggesting an extremely low rate of loss in this mutation category (clonal multi-copy mutations: 4 out of 1031 lost, 0.4% loss frequency, clonal only-copy mutations: 1 out of 117 lost, 0.9% loss frequency, FIG. 12) and an odds ratio for the difference in the loss frequency persistent and loss-prone mutations of 61.43 (P<2.2e-16). Collectively, our new analyses provided further proof of the contribution of persistent mutations in clinical outcomes with ICB and further supported the robustness and biological basis or persistent mutations that are retained in the course of tumor evolution.

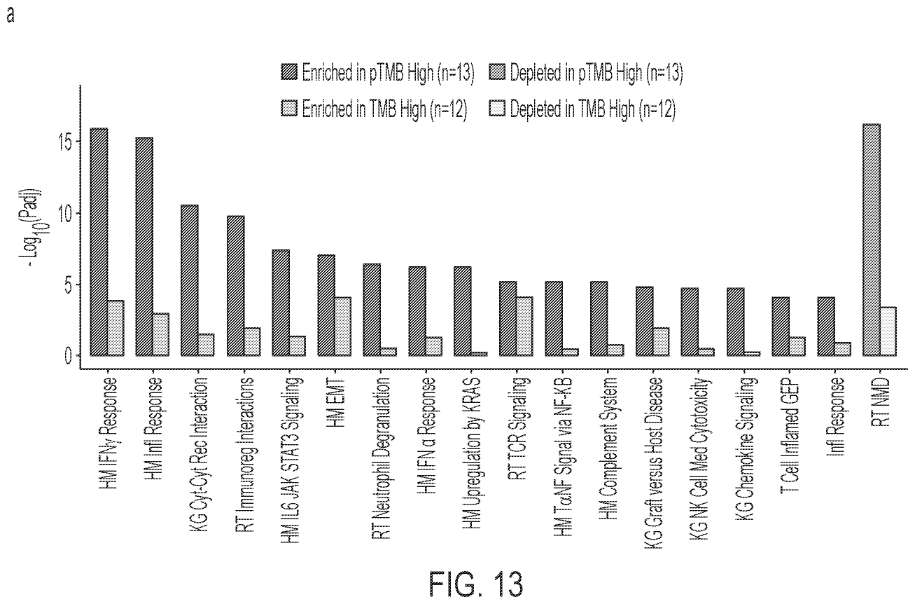

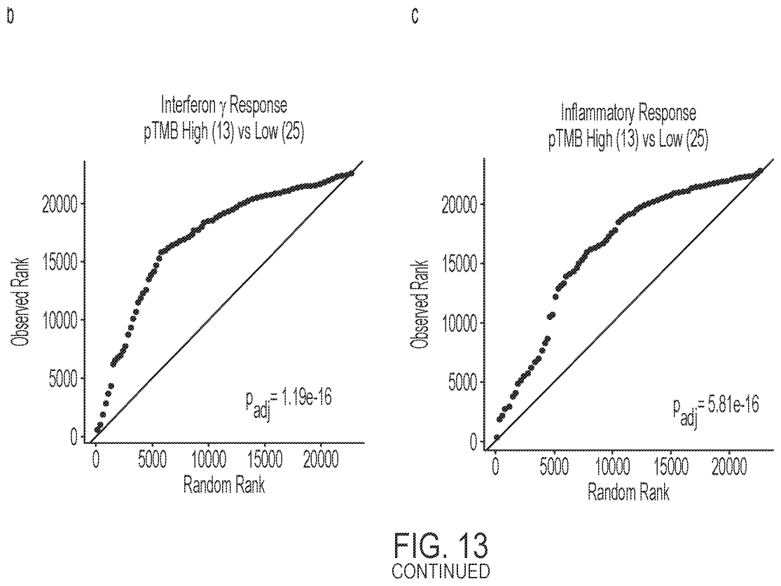

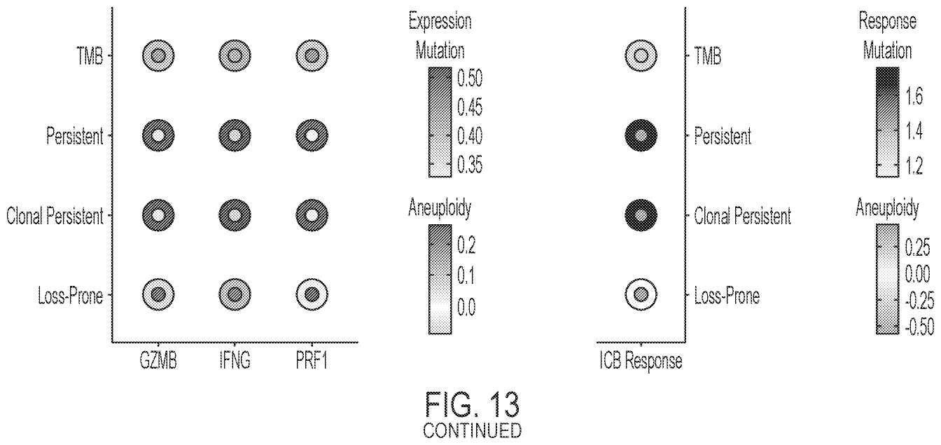

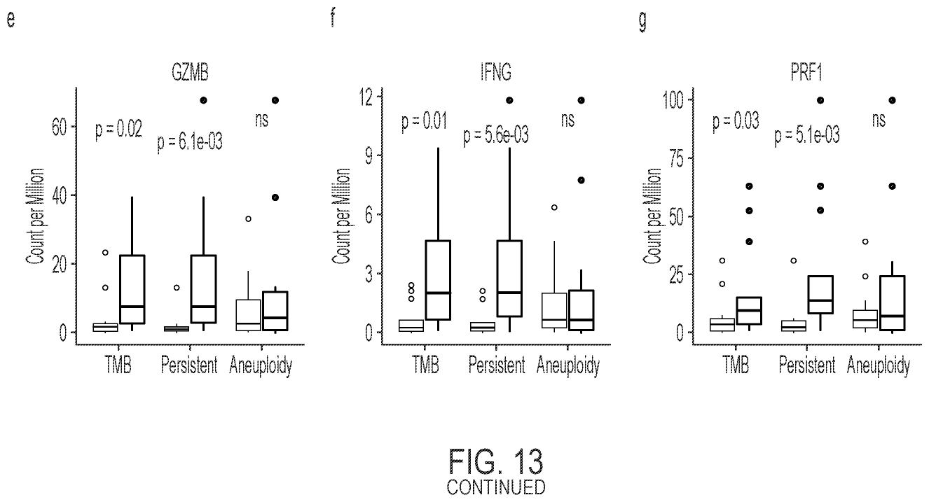

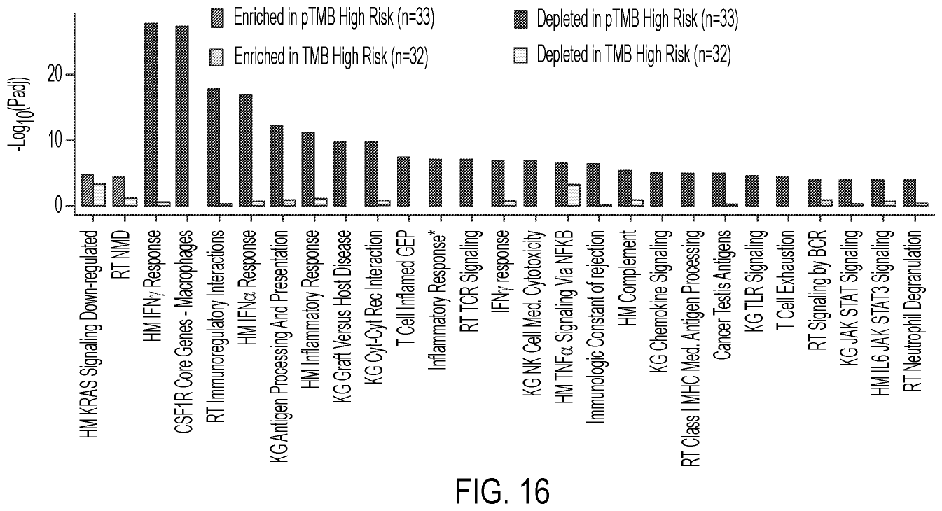

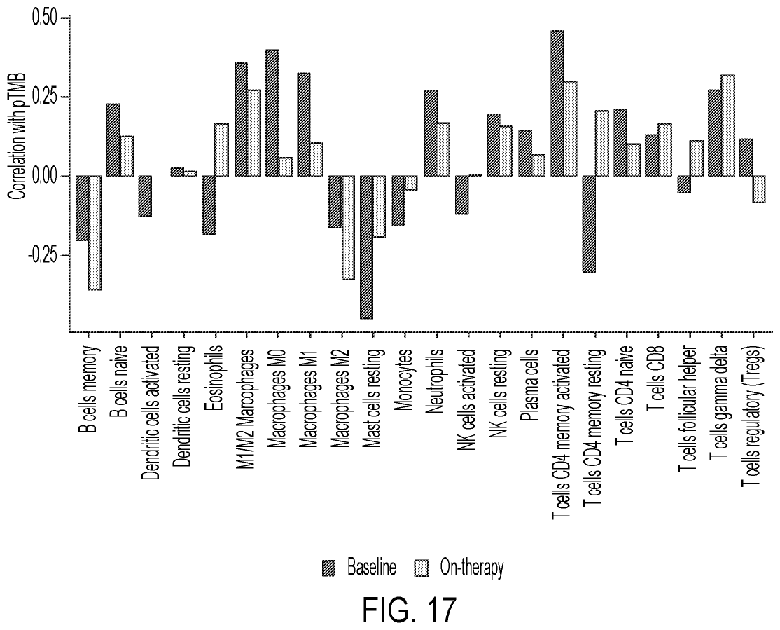

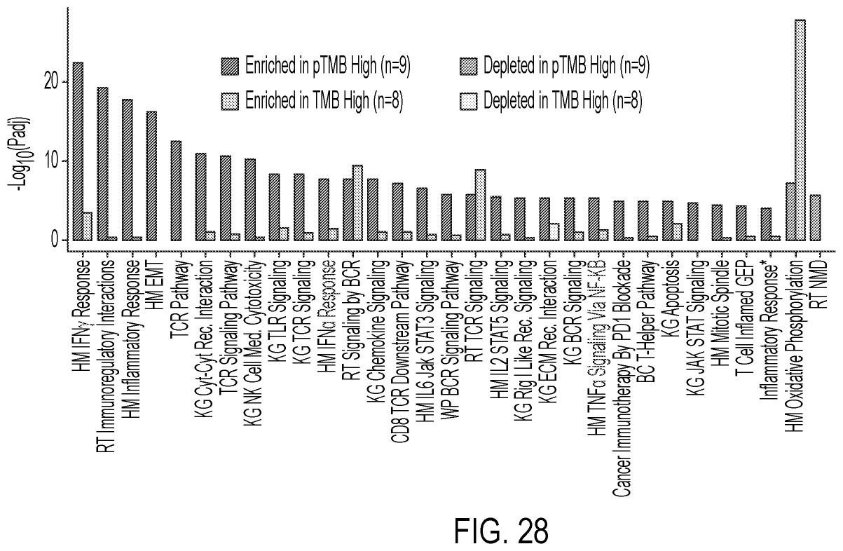

Shifting our focus from the tumor to the tumor microenvironment (TME), we explored transcriptomic profiles in the TME of IC-treated tumors and postulated that a high pTMB would generate an un-interrupted feed of neoantigens that would in turn trigger interferon-γ signaling and adaptive immunity cascades that may be enhanced with ICB. To this end, serial RNA sequencing analyses of ICB-treated melanomas¹⁴ (Methods) revealed a marked enrichment in interferon-γ and inflammatory response related gene sets prior to therapy (FIG. 13A-C) that was significantly enhanced during ICB (FIG. 28), suggesting robust adaptive immune responses in the TME of melanomas with high pTMB. Notably, the differential enrichment in pro-inflammatory gene sets was significantly lessened in tumors stratified by their TMB content (FIG. 13A and FIG. 28). Similar trends were observed in transcriptomic analyses comparing the TME of melanoma tumors in the TCGA set that were stratified by pTMB compared to TMB (FIG. 16). Leveraging RNA sequencing data deconvolution, we explored the relationship between pTMB and abundance of key immune cell subsets in the TME. In addition to positive correlations between pTMB and CD8 and CD4 T cell abundance (FIG. 17), the ratio of M1 to M2 macrophages was significantly higher in pTMB-high tumors in both baseline (Spearman's ρ=0.36) and on-therapy samples (Spearman's ρ=0.28), further highlighting the inflamed phenotype of pTMB-high tumors

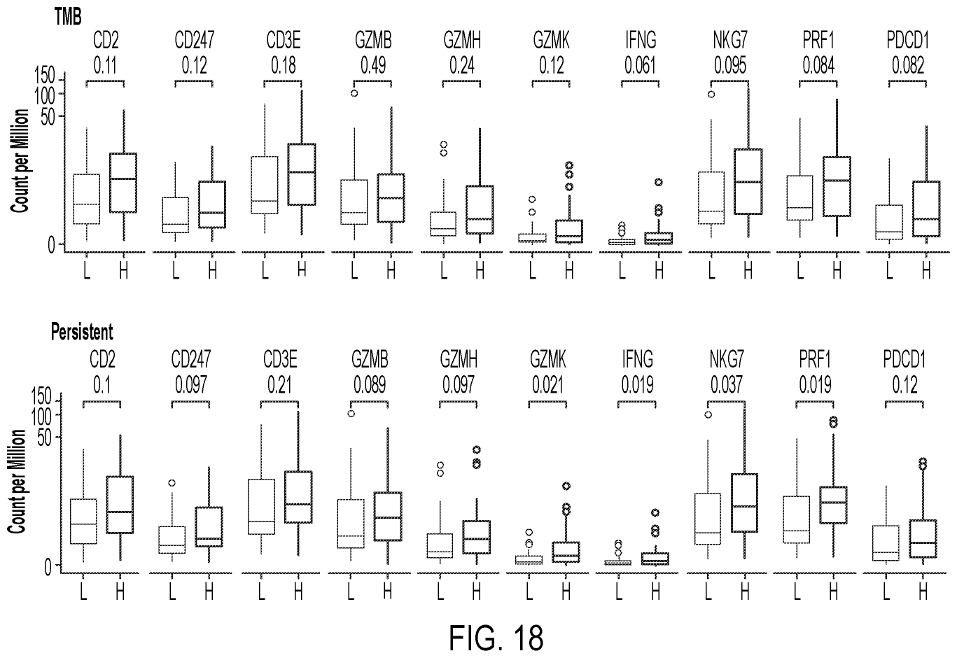

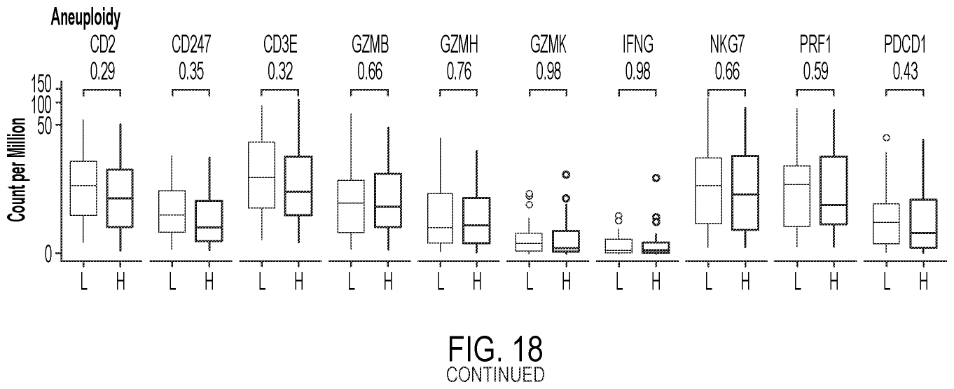

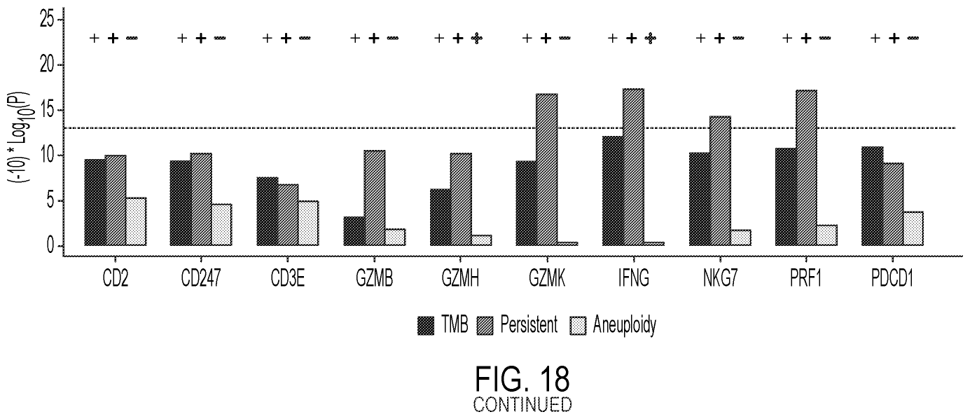

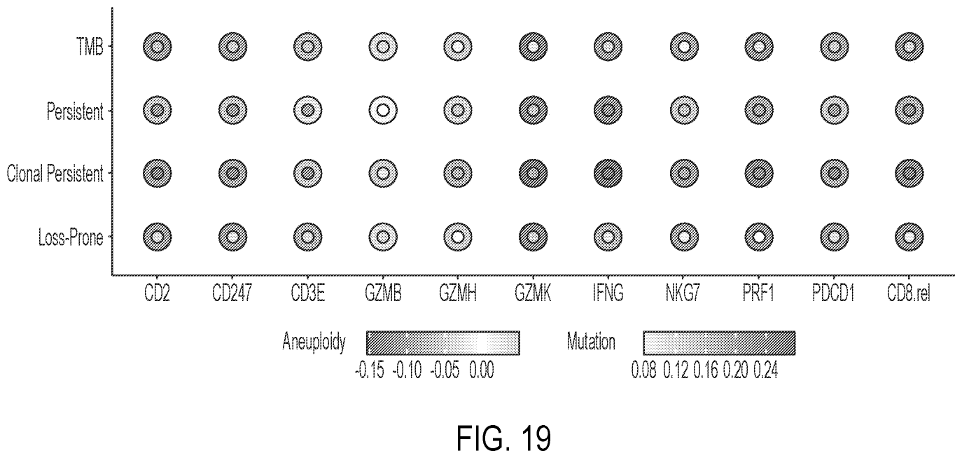

We further dissected integrated whole exome and RNA sequencing data in the TCGA and ICB melanoma¹⁴ cohorts to investigate the relationship of pTMB, tumor aneuploidy and TME immune phenotypes. To this end, we compared the expression of genes representing cytolytic activity between tumors in the top and bottom tertile of TMB, pTMB, and aneuploidy (Methods). We found a higher expression of cytolytic markers in pTMB-high compared to TMB-high or aneuploidy-low tumors in both the TCGA (TMB p >0.05 for all genes; pTMB p=0.02 for GZMK, IFNG, and PRF1. p=0.04 for NKG7, aneuploidy p>0.05 for all genes, FIG. 18) and ICB melanoma cohort (GZMB: TMB p=0.02, pTMB p=6.1e-03, aneuploidy p>0.05; IFNG: TMB p=0.01, pTMB p=5.6e-03, aneuploidy p >0.05; PRF1: TMB p=0.03, pTMB p=5.1e-03, aneuploidy p>0.05; FIG. 13). We then modeled the expression of key cytolytic genes based on pTMB and aneuploidy using multivariable regression; for the ICB melanoma cohort, we also modeled patients' therapeutic response. These analyses showed that a high value of pTMB counteracts the negative (but not statistically significant) impact of aneuploidy on cytolytic activity and ICB response to ICB (FIG. 13 and FIG. 19).

Taken together, our analyses suggested that a high persistent mutation burden, which comprises a biologically relevant measure of tumor foreignness within the overall TMB, would represent an “uneditable” target set for adaptive immune responses (FIG. 20). This hypothesis relies on the basis that pMANAs are less likely to be eliminated by chromosomal loss due to the intrinsic fitness cost to the tumor and therefore may mediate sustained neoantigen-driven immune responses and long-term clinical benefit. To further explored this, we performed integrative analyses incorporating measures of MANA expression, MHC binding affinity, and persistence in the context of ICB response (Methods). Similar to pTMB, pMANA load and importantly expressed pMANA load distinguished responding from non-responding tumors (melanoma, n=42, pTMB p=2.51E-04, pMANA p=3.37E-04, expressed pMANA p=2.91E-04; NSCLC, n=74, pTMB p=1.26E-04, pMANA p=1.10E-04, expressed pMANA p=1.15E-04; Mann Whitney U-test). We did not observe a further improvement of pMANA performance by restricting our analyses to the subset of MANAs with computationally inferred high MHC class I binding affinity¹⁵, which is highlighting the limitations with of MANA predicting algorithms in identifying biologically relevant neoepitopes. To this end, we sought to generate additional functional proof that pMANAs are indeed recognized and elicit epitope-specific T cell expansions and pulsed autologous T cells from a patient with NSCLC with peptides synthesized based on MANAs independent of whether these are derived from persistent or loss-prone mutations. All but one peptides that elicited TCR clonotypic expansions were encoded by persistent mutations, suggesting that pMANAs are recognized by CD8+ T cells and trigger a pMANA-specific immune response and further supporting the biological importance of persistent mutations.

### Discussion

Since the first reports recognizing TMB as a predictor of clinical response to immune checkpoint blockade in melanoma and non-small cell lung cancer^{16,17}, it has become clear that TMB as a numeric value or binarized feature can only partially predict response to immune checkpoint blockade. Our findings suggest that a high pTMB, a biologically relevant measure of tumor foreignness within the overall TMB, represents an “uneditable” target set for adaptive immune responses and may function as an intrinsic driver of sustained immunologic tumor control that cannot be readily bypassed by neoantigen loss via chromosomal deletions during cancer evolution.

Similar to TMB, that is linked with response to immunotherapy in a dose-dependent and cancer lineage-specific manner¹⁸, pTMB has to be considered in the context of the background aneuploidy rate within a specific tumor type. What we have learned from the increasing number of studies evaluating the overall TMB in predicting therapeutic response with ICB is that using a fixed pan-cancer threshold for a biomarker with different distributions and dynamic ranges depending on cancer lineage¹⁹ can be challenging and may miss up to 25% of ICB-responsive tumors²⁰. In our current work and in order to avoid these challenges, we evaluated both persistent mutations and TMB as continuous variables in the context of response to immune checkpoint blockade, thus our findings are less susceptible to artefactual associations resulting from application of a threshold. To expand our analyses in supporting a biologically distinct role for pTMB that is reflected in therapeutic response difference compared to overall TMB-based classifications, we evaluated the number of tumors with differential pTMB/TMB classification within the melanoma ICB cohort and found a higher response rate among tumors reclassified by their pTMB content. Notably, the relative contribution of multi- and only-copy mutation components to the overall pTMB varies across cancer lineages. This pattern appears to be driven by the dominant copy number state of the tumor; suggesting that the dominant copy number state has to be considered together with the sequence alteration load affecting these genomic regions.

The premise of pTMB relies on the potential of pMANAs to mediate sustained neoantigen-driven immune responses. Overall MANA burden has failed to demonstrate an incremental value over TMB in predicting clinical outcomes with immune checkpoint blockade. Nevertheless, it is the neoantigen quality not the quantity that may be most informative in predicting therapeutic response²¹⁻²³. Another feature that may determine the role of mutation-associated neoantigens in anti-tumor immune responses is expression, as expressed single base substitution-derived neopeptides have been shown to more accurately predict response to ICB compared to TMB¹⁴. In line with this notion, the significance of mutant protein abundance in driving T cell responses may further support the importance of multi-copy persistent mutations, as their presence at higher number of copies per cell likely correlates with higher expression of mutant mRNAs and proteins. We indeed found a marginal improvement in outcome prognostication of pMANA burden compared to pTMB in ICB treated NSCLC. Importantly, by testing pMANA-specific TCR clonotypic expansions in vitro, we provide proof that pMANAs can elicit memory T cell responses that are likely to drive tumor elimination.

Placing pTMB in context of other genomic features that have been associated with response to ICB¹, we assessed the clonal architecture of persistent mutations and considered the potential confounding effect of tumor clonal heterogeneity. Overall in the TCGA and ICB cohorts, the cellular fractions of persistent mutations did not differ from loss-prone mutations; notably, persistent mutations in the multi-copy category tended to be more clonal in a cancer lineage-dependent manner, suggesting that these may have been acquired earlier in tumor evolution prior to the copy number gain event. Persistent mutations more optimally distinguished responding from non-responding tumors compared to clonal TMB in all ICB cohorts analyzed. Importantly, clonal persistent mutations were more significantly associated with response in the ICB-treated NSCLC cohorts. These findings suggest that persistent mutation content is distinct from a tumor's clonal heterogeneity and considering these features together may be most informative in predicting response to immunotherapy.

While pTMB is related to tumor aneuploidy and a higher degree of large-scale chromosomal changes has been reported in ICB non-responsive tumors¹³, in the ICB cohorts analyzed tumor aneuploidy alone failed to differentiate responding from non-responsive tumors. While our analyses did not show a strong association between tumor aneuploidy or whole-genome doubling and response to ICB as individual predictive biomarkers, the number of persistent mutations was correlated with tumor aneuploidy, and we found an enrichment for multi-copy persistent mutations in tumors with whole-genome doubling. Our findings highlight the importance of measuring mutational burden in regions of the genome with structural changes rather than considering overall TMB or tumor aneuploidy independently.

Importantly, we studied the evolution of persistent mutations in the evolutionary trajectories shaped by selective pressure of ICB. We hypothesized that multi-copy mutations would inherently be more difficult to lose, as the process of loss would require multiple distinct genomic events and similarly, single-copy mutations are unlikely to be lost by chromosomal deletions as these may be detrimental to the cancer cell; rendering persistent mutation loss not biologically plausible. Consistent with this notion, we discovered that persistent mutations are retained in the context of tumor evolution while losses predominantly affect loss-prone mutations. While approximately 99% of mutations lost in serial analyses of NSCLC during ICB were loss-prone mutations, four clonal multi-copy persistent mutations were not detected in comparative analyses of baseline/ICB resistant tumors, which may be explained by presence of the multiple copies of a mutation in tandem or in close proximity on a common chromosomal segment, thus the loss of multiple copies could be achieved by a single genomic event.

Taken together our findings suggest that mutations located in single-copy regions or these present in multiple copies in the cancer genome are unlikely to be lost under the selective pressure of immunotherapy due to the intrinsic fitness cost to the tumor and therefore serve as a key driver of sustained immunologic tumor control.

### Methods

### Cohorts

We evaluated 10,742 tumor samples from TCGA and 485 non-small cell lung cancer, melanoma and mesothelioma tumor samples from published cohorts of patients that received immune checkpoint blockade^{2,4,6-9,24}. Patients with melanoma across 4 source studies^{6-8,22} were combined to generate an aggregated melanoma cohort (n=202). Clinical outcomes were retrieved from the original publications. We further performed whole exome sequencing analyses for a cohort of 39 patients with HPV negative (HPV−) HNSCC who received ICB at University of Chicago (HNSCC Cohort). We assessed serially sampled NSCLC tumors from 8 patients from a published study³ as well as from 4 patients with NSCLC who received ICB at the Nederlands Kanker Instituut (NKI set). The studies were conducted in accordance with the Declaration of Helsinki and were approved by the Institutional Review Board (IRB), and patients provided written informed consent for sample acquisition for research purposes.

### Whole Exome Sequencing and Sequence Data Processing

DNA extraction and genomic library preparation were performed as previously described². The coding sequences were captured in solution using the SureSelect XT Human All Exon V6 kit in the HNSCC cohort, and using the SureSelect Human All Exon V4 kit in the NKI cohort. Whole exome sequencing derived multi-center mutation calls from the TCGA pan-cancer atlas²⁵ were retrieved from the NCI Genomic Data Commons (https://gdc.cancer gov/about-data/publications/mc3-2017) and filtered to keep nonsynonymous alterations. For the ovarian cancer (OV) tumor type, indels were excluded from all downstream analyses to minimize technical artifacts²⁶. Somatic copy number profiles including estimates of tumor purity and ploidy, as well as allele-specific copy number states²⁷ were acquired via the pan-cancer atlas (https://gdc.cancer.gov/about-data/publications/pancanatlas). Clinical annotations of tumors were accessed using the TCGA clinical data resource²⁸. For the HNSCC subset in TCGA, HPV status was retrieved from eBioPortal (https://www.ebioportal.org/study/summary?id=hnsc_tega_pan_can_atlas_2018) Analyses of copy number profiles to establish the background rate of genomic loss were performed on 10,742 tumor samples where the segmental allele-specific copy numbers were available. For a subset of 9,242 tumor samples from the above, both somatic mutation calls and copy-number profiles were available, enabling assessment of persistent mutations. For a subset of 8,925 tumors where clinical data annotations including overall survival and tumor stage assessment were available, survival analyses evaluating the contribution of persistent mutations were performed.

For the publicly available published ICB cohorts, we retrieved allele-specific copy number profile, tumor purity and ploidy estimates, as well as somatic mutation calls, raw gene expression counts, and clinical annotations of response to treatment from the original publications. Furthermore, for the Riaz et al., melanoma cohort⁶ allele-specific somatic copy number profile, and tumor purity and ploidy estimates were generated by application of FACETS to tumor and matched normal sequence data²⁹. For the Liu et al. melanoma cohort⁷ the short read archive files were accessioned from SRA and this sample set was filtered to only keep tumors with no prior anti-CTLA4 treatment. Adaptor sequences were detected and trimmed using FASTP³⁰. Sequenced reads were aligned to the reference genome assembly hg19 using bowtie2³¹, and duplicate reads marked by sambamba³². Tumor purity and ploidy estimates, as well as somatic copy number profiles were derived by application of FACETS²⁹ to tumor and matched normal pairs. For the Hugo et al. melanoma cohort⁸, fastq files were obtained from the SRA. Sequencing read processing and alignment were performed as described for Liu et al. cohort, and copy number profiles were similarly obtained by application of FACETS. For the Shim et al., NSCLC cohort⁹, somatic mutations were narrowed down to those with mutant allele fraction greater than or equal to 10% to minimize sequencing artifacts. Tumor purity and ploidy estimates, and somatic copy number profiles were generated by application of FACETS²⁹ to tumor and matched normal pairs. For the HNSCC cohort, somatic mutations were identified using the Strelka mutation calling pipeline³³. Mutations in common SNP locations (dbSNP v138) and greater than one BLAT³⁴ hit were filtered out. The final set of mutations were obtained after filtering for tumor mutant allele fraction >=10%, normal mutant allele fraction <=3% and matched normal coverage >=11×. For samples from the NKI set, sequence read processing and alignment were performed as previously described². Tumor purity and ploidy estimates and somatic copy number profiles were derived by application of FACETS. While we did not have uniform documentation of MSI in the IO cohorts analyzed, the very low background prevalence of MSI-high tumors in NSCLC (<1%), melanoma (<1%), mesothelioma (˜2%), and HNSCC (<1%)³⁵ renders MSI an unlikely confounder in this study.

### Evaluation of Mutation Multiplicity and Cancer Cell Fraction

Mutation cellular fractions were estimated as previously described^{3,36}. Considering the tumor sample purity α, tumor copy number n_{T}, and normal copy number n_{N}, the expected variant allele fraction Vₑₓₚ for a mutation at cellular fraction C with multiplicity m (i.e. m mutant copies per cancer cell) can be calculated as

$${V}_{exp} = \frac{m C α}{α {n}_{T} + ( 1 - α ) {n}_{N}}$$

The purity and segmental tumor and normal copy numbers were determined via genome-wide analysis of sequencing coverage distribution and b-allele frequency of heterozygous SNPs in each cohort. Assuming a binomial distribution for the number of reads harboring the mutant allele, a 95% confidence interval (CI) is constructed for Vₑₓₚ using the distinct total coverage and mutant read counts for each mutation (i.e. coverage and read counts after exclusion of reads marked as duplicates). Since estimates for α, n_{T}, and n_{N} are available, this yields a 95% CI for the product of mutation cellular fraction C and multiplicity m. By application of the following rules, one can derive estimates for C and m: (1) If the confidence interval for m C contains an integer, the mutation is deemed clonal and that value is assigned to the multiplicity. (2) If the entire CI is below 1, multiplicity is assumed to be 1 and the mutation is subclonal except cases where it is within a tolerance threshold of 1 (C>0.75). (3) For a CI that is entirely above 1 and does not include any integer, m is greater than one and is assigned such that the CI falls within the expected range [0, 1]. Mutation clonality can now be calculated using the rule in (2).

### Assessment of Single-Copy, Multi-Copy, and Persistent Tumor Mutation Burden

The nonsynonymous somatic mutations in each tumor were intersected with the segmental integer copy number profile to assign minor and major copy number states to the mutated loci. Mutation multiplicity (number of mutated copies per cell) and cancer cell fraction (proportion of cancer cells harboring the mutation) were estimated based on the mutant read count, total coverage, tumor purity, and the major and minor allele-specific copy number in the tumor and normal compartments for each mutation. Mutations present in more than one copies per cancer cell constituted the multi-copy category. Those present in regions of the genome with a single copy (total copy number=1) were included in the single-copy category. The persistent tumor mutation burden was defined as the number of mutations in either multi-copy or single-copy category. For mesotheliomas, given the predominance of copy number losses^{4,37}, the persistent mutation burden was limited to mutations within single-copy regions of the genome. Furthermore, to assess the differential potential of persistent mutation in predicting outcome compared to TMB, we defined the number of loss-prone mutations in each tumor sample as the difference between the total number of mutations assessed (excluding mutations on sex chromosome or those at loci without copy-number assignment) and the number of persistent mutations. Finally, to achieve harmonized comparisons, mutations on sex chromosomes or on loci lacking allele-specific copy number assignment were excluded from analyses.

### Characterization of Tumor Aneuploidy

Aneuploidy metrics were calculated for tumor samples from TCGA and ICB cohorts, and their relationship with persistent mutation burden was characterized. Furthermore, in IO-treated cohorts, aneuploidy metrics were also considered as independent predictors of outcome. The fraction of genome with allelic imbalance (AI) was calculated as a broad metric summarizing the aneuploidy level across the autosomes. We also considered the fraction of genome with single copies (total copy number of 1) and the fraction of genome with multiple copies (i.e. major allele-specific copy number greater than 1) given their direct link to persistent mutation burden. Tumor samples with more that 50% of the autosomal length at major allele-specific copy number of 2 or above were marked as having undergone whole-genome doubling (WGD)³⁸.

### Evaluation of the Background Rate of Genomic Loss

To evaluate the background rate of genomic loss, we analyzed the somatic copy number profiles of 10,742 samples from TCGA. In each tumor sample, the chromosome arms in diploid state were defined as those where 75% of the length of segments covering the arm was copy neutral (total copy number of 2) and did not harbor loss of heterozygosity (LOH). The chromosome arms in haploid state had 75% of their length covered by segments with a total copy number of 1. The rate of loss in diploid regions of the genome was defined as R_{D}

$${R}_{D} = \frac{2 * {l}_{D}^{HD} + {l}_{D}^{HM}}{2 * {l}_{D}}$$

Where Ipindicates the total length of segments in arms of diploid state, l_{D}^{HD} is the total length of the segments in homozygous deletion in diploid arms, and l_{D}^{HM} is the total length of the segments with single copy loss in diploid arms. Similarly, the rate of loss in haploid regions of the genome was defined as R_{H}

$${R}_{H} = \frac{{l}_{H}^{HD}}{{l}_{H}}$$

Where l_{H} is the total length of segments in haploid arms and l_{H}^{HD} is the total length of segments with homozygous deletion in those arms. Comparison of the background rates of genomic loss was performed on subset of the TCGA tumor samples where at least one chromosome arm was found in each of diploid and haploid states (n=5,244).

Evaluation of pTMB Quantification by Gene Panel Targeted NGS

To determine the feasibility of using clinical targeted NGS to estimate persistent tumor mutation burden, we performed in silico simulations as follows. Given the inherent limitation of targeted NGS in identification of mutations in tumor types with low TMB, we performed a focused analysis in melanoma and NSCLC cohorts. We assumed that allele-specific copy number estimates could be derived by targeted NGS as previously shown^{29,39}. Therefore, we focused our analysis on the subset of mutations that would be captured by the genomic intervals contained in FoundationOne CDx, which is a widely used clinical targeted NGS panel. The list of 309 genes with their full coding sequence included in FoundationOne CDx panel was retrieved from the FDA website at https://www.accessdata.fda.gov/cdrh_docs/pdf17/P170019S006C.pdf. The RefSeq Select transcript set was used to determine the genomic coordinates of the coding exons for each gene. Mutations in each tumor sample were intersected with panel coordinates to determine simulated estimates of TMB and pTMB as captured by the panel.

### Differential Expression and Gene Set Enrichment Analysis

Expression counts from RNA sequencing of pre-treatment melanoma tumors from the CM038 melanoma cohort were retrieved from the original publiation²⁴. Differential expression testing was performed using DESeq2⁴⁰ and the resulting p-values were corrected for multiple testing using the Benjamini-Hochberg procedure. For the TCGA tumor type SKCM, the TCGAbiolinks R package⁴¹ was used to download harmonized raw RNAseq counts data from the NCI Genomic Data Commons within the target cancer type. This sample set was then narrowed down to the set of samples with persistent mutation estimates and available overall survival data, and comparisons were performed between samples within the top tertile of PTMB/TMB-informed risk (high risk) vs the remaining set (low risk). For gene set enrichment analysis, each gene which passed the count threshold was ranked by −log(p)*sign(fc) where p is p-value and fc is fold-change, resulting to ranking where the genes on each flank represent the mostly statistically significantly up- or down-regulated genes and the genes in the middle are the least significant. Gene set enrichment analysis (gsea) was then performed using the fgsea R package⁴² with a curated list of gene sets from the Molecular Signatures Database related to immune responses and cancer hallmarks. Tumors were classified into high or low groups for TMB and pTMB using the 2^{nd} tertile value. The complete list of gene sets contains the gsea results for comparisons based on persistent mutation burden (pTMB) and TMB in baseline and on-treatment samples. The p-values for gsea were corrected for multiple testing with the Benjamini-Hochberg procedure. Quantile-quantile plots were made to provide a visual comparison of the ranks of pathway genes to a set of ranks sampled from the background distribution.

### Modeling of Cytolytic Activity

Gene level expression values (in Count Per Million) were used from the CM038 IO melanoma cohort²⁴ and TCGA melanoma cohort. In each cohort, expression levels for a selected set of gene markers of cytolytic activity were compared between tumors in the top and bottom tertiles of a number of key variables of interest using Mann Whitney U-test. Furthermore, a multivariable linear regression model defined the combined contribution of a mutation based marker (i.e. pTMB, TMB, etc) and aneuploidy (as measured by the fraction of genome with allelic imbalance) to cytolytic activity. Briefly, both mutation based marker values and gene expression levels were pseudo log transformed to control the right skew in the distribution. Next, each variable was scaled to have zero mean and unit variance over the analyzed cohort. In each regression model, predictor coefficients and the associated p-values were recorded. In the IO melanoma cohort, multivariable logistic regression was used to model the contribution of mutation based markers and aneuploidy. In addition, estimates for the relative abundance of 22 immune cell subpopulations derived by CIBERSORT v1.06 were retrieved from the earlier publication. For the TCGA melanoma tumors, the relative abundance of CD8 T cells were retrieved from the genomic data commons⁴³.

### Longitudinal Tracking of Persistent Mutations

For the 8 NSCLC patients with serially biopsied tumor samples, tumor samples were acquired prior to ICB and at the time of acquired resistance; for all cases a minimum of 6 months lapsed between ICB initiation and re-biopsy in the setting of acquired resistance. For each patient, the set of mutations identified in the baseline sample was annotated with distinct total coverage, distinct mutant read count, and minor and major allele-specific copy numbers. These annotations were combined with the estimated purity of the tumor sample to yield estimates of mutation cancer cell fraction and multiplicity. The combination of copy number assignment and multiplicity estimate for each mutation in the baseline sample enabled identification of only-copy, multi-copy, and persistent mutations, as well as those prone to loss (loss-prone). Mutations identified in the baseline sample with mutant allele fraction of zero at the time of progression were deemed lost.

### Analysis of Mutation Associated Neoantigens

An integrated analysis of persistent mutation associated neoantigens was performed in the ICB NSCLC² and melanoma²⁴ cohorts. Briefly, MANA predictions by ImmunoSelect-R pipeline (Personal Genome Diagnostics, Baltimore, MD) were retrieved from the original studies. The set of predicted peptides were restricted to those with predicted MHC class I binding affinity (IC50) less than 500 nM, and mutations with at least one associated peptide were marked as MANA-encoding. Mutations in genes with non-zero median expression in the respective TCGA tumor type were marked as expressed.

### Assessment of Replication Timing for Persistent Mutations

We compared persistent and loss-prone mutations with regards to the replication timing of the mutated loci in melanoma (TCGA-SKCM) and NSCLC (TCGA-LUAD, TCGA-LUSC) tumors from TCGA. We retrieved replication timing scores for NHEK (skin) and IMR90 (lung) cell lines, which were measured by Repli-Seq methodology as part of the ENCODE project, using the UCSC table browser. In each cell line, we used scores from the “wavlet-smoothed signal” track, which is the result of application of a wavelet smoothing transformation to the weighted average of the percentage-normalized signals in 1 kb intervals across the genome, where higher values indicate earlier replication timing. Genomic intervals were marked based on their quintile membership, and the frequency of persistent and loss-prone mutations across the quintiles were visualized. Replication timing of persistent and loss-prone mutations were compared by evaluating Cohen's d effect size.

### Analysis of NGS Technical Limitations

To assess the possibility of technical artifacts preferentially impacting the somatic mutation calls in our pan-cancer analysis of 31 tumor types, we determined the prevalence of persistent and loss-prone mutations identified in regions of the genome susceptible to limitations of NGS analysis. The UCSC Table Browser was used to retrieve the “Problematic Regions” track, including regions marked by ENCODE¹¹, Genome-In-A-Bottle¹² and NCBI GeT-RM⁴⁴.

### Functional T Cell Assays

The MANAFEST (Mutation-Associated NeoAntigen Functional Expansion of Specific T Cells) assay³ was employed determine MANA-specific T cell clonotypic expansions in the peripheral blood of a patient with NSCLC that attained long progression-free and overall survival with immune checkpoint blockade (CGLU310). Briefly, candidate neopeptides (JPT Peptide Technologies) were synthesized and each used to stimulate and co-culture T cells in vitro as previously described. On day 10, cells were harvested and the CD8+ fraction was isolated using a CD8+-negative enrichment kit (EasySep, STEMCELL Technologies), followed by DNA extraction from each CD8-enriched culture condition. TCR VB CDR3 sequencing was performed by the SKCCC FEST and TCR Immunogenomics Core (FTIC) on genomic DNA from each T cell condition using the Oncomine TCR Beta short-read assay (Illumina) as previously described¹⁰. Following data pre-processing, alignment and trimming, productive frequencies of TCR clonotypes were calculated. To be considered antigen-specific, a T cell clonotype must have met the following criteria: (1) significant expansion (Fisher's exact test with Benjamini-Hochberg correction for FDR, P<0.05) compared to T cells cultured without peptide; (2) significant expansion compared to every other peptide-stimulated culture (FDR<0.05); (3) an odds ratio greater than 5 compared to all other conditions; (4) at least 30 reads in the ‘positive’ well; and (5) at least 2× higher frequency than background clonotypic expansions as detected in the HIV-negative control condition.

Evaluation of Differentially Classified Tumors by TMB and pTMB

Re-classification rates based on pTMB vs TMB were computed as follows: in each tumor type and for each variable (TMB and pTMB), a series of quantile values ranging from 5% to 95% in 5% increments were applied to define samples with high and low values for that variable. Next, at each quantile value, we calculated the rate at which sample classification differed by the two metrics (i.e. the combined prevalence of samples that were pTMB-low, TMB-high and samples that were pTMB-high, TMB-low within that tumor type for that quantile threshold) to derive the re-classification rate. In the ICB cohorts, we determined the cases with differential PTMB/TMB classification and compared the therapeutic response rates between pTMB-low/TMB-high and pTMB-high/TMB-low tumors. For each predictor variable (pTMB or pTMB), the second tertile was used to determine tumor samples with high and low values. Given the current cohort sizes, sufficient sample size for this comparison was only available in the combined melanoma cohort where 23 samples were labeled as TMB-high/pTMB-low and another 23 samples were marked as TMB-low/pTMB-high.

### Survival Analysis of TCGA Tumors

The relationship of pTMB and TMB with overall survival was assessed in 8,925 patients from 31 cohorts in TOGA. Tumor types with more than 50 informative samples were analyzed and overall survival was selected as an endpoint. Briefly, in each tumor type and stage combination, Cox proportional-hazards (CoxPH) models were constructed for each one of the following features as independent continuous predictors: TMB, persistent mutations-pTMB, clonal pTMB, multi-copy mutations, clonal multi-copy mutations, only-copy mutations, clonal only-copy mutations (continuous CoxPH model). In cases where an increase in the predictor variable was associated with better outcome (longer survival), a second CoxPH model is used to assess the difference in overall survival between tumors in the top third and bottom two thirds of predicted risk (categorical CoxPH model).

### Statistical Analyses

For the TCGA cohort, in each tumor type, a cox proportional hazard's model was used to evaluate the contribution of TMB and pTMB to overall survival. The predicted risk values from these models were then used to stratify the tumors into low and high-risk groups using the second tertile of the predicted risk as the threshold. For the immunotherapy cohorts, clinical response assessments were retrieved from the original publications. The Mann-Whitney U test was used to evaluate the difference of continuous variables between groups, including the differences of predictive variables between responding and non-responding tumors, and the difference of background rate of loss between haploid and diploid regions of the genome. Cohen's d statistic was used to quantify the effect size of each predictor variable in the ICB cohorts. Fisher's exact test was used to assess the association of dichotomous variables (such as whole-genome doubling) with therapeutic response. The association of the fraction of mutations in single and multi-copy regions with TMB ranks was evaluated by the Pearson correlation coefficient, while non-parametric correlations were evaluated by the Spearman correlation coefficient. The Kaplan-Meier method was used to estimate the survival function and the survival curves were compared using the non-parametric log-rank test. All p values were based on two-sided testing and differences were considered significant at p<0.05. Statistical analyses were done using R version 3.6 and higher, http://www.R-project.org/)

### Data Availability

Source data for the TCGA tumor samples were retrieved from http://cancergenome.nih.gov. WES-derived somatic mutation calls from the TCGA PanCancer Atlas MC3 project were retrieved from the NCI Genomic Data Commons (https://gdc.cancer.gov/about-data/publications/mc3-2017). Previously published genomic data, re-analyzed here, were obtained from the material of the original publications and from dbGaP under accession code phs000452.v3.p1⁷, and Sequence Read Archive (SRA) under accession codes SRP095809⁶, SRP067938⁸ and SRP090294⁸. WES sequence data for the HNSCC and NKI cohorts from patients who consented to data deposition can be retrieved from the European Genome-phenome Archive (EGA accession number EGAS00001006660).

### REFERENCES

1. Litchfield, K. et al. Meta-analysis of tumor- and T cell-intrinsic mechanisms of sensitization to checkpoint inhibition. Cell 184, 596-614 e514 (2021). https://doi.org:10.1016/j.cell.2021.01.002
2 Anagnostou, V. et al. Multimodal genomic features predict outcome of immune checkpoint blockade in non-small-cell lung cancer. Nature cancer 1, 99-111 (2020). https://doi.org:10.1038/s43018-019-0008-8
3 Anagnostou, V. et al. Evolution of Neoantigen Landscape during Immune Checkpoint Blockade in Non-Small Cell Lung Cancer. Cancer discovery 7, 264-276 (2017). https://doi.org:10.1158/2159-8290.CD-16-0828
4 Forde, P. et al. Durvalumab with Platinum-Pemetrexed for Unresectable Pleural Mesothelioma: Survival, Genomic and Immunologic Analyses from the phase 2 PrE0505 trial Nat Med (2021). https:/doi.org:https://doi.org/10.1038/41591-021-01541-0
5 Leary, R. J. et al. Integrated analysis of homozygous deletions, focal amplifications, and sequence alterations in breast and colorectal cancers. Proceedings of the National Academy of Sciences of the United States of America 105, 16224-16229 (2008). https://doi.org:10.1073/pnas.0808041105
6 Riaz, N. et al. Tumor and Microenvironment Evolution during Immunotherapy with Nivolumab. Cell 171, 934-949 e916 (2017). https://doi org: 10.1016/.cell.2017.09.028
7 Liu, D. et al. Integrative molecular and clinical modeling of clinical outcomes to PD1 blockade in patients with metastatic melanoma. Nat Med 25, 1916-1927 (2019). https://doi.org:10.1038/s41591-019-0654-5
8 Hugo, W. er al. Genomic and Transcriptomic Features of Response to Anti-PD-1 Therapy in Metastatic Melanoma. Cell 165, 35-44 (2016). https://doi.org:10.1016/j.cell.2016.02.065
9 Shim, J. H. et al. HLA-corrected tumor mutation burden and homologous recombination deficiency for the prediction of response to PD-(L)1 blockade in advanced non-small-cell lung cancer patients. Ann Oncol 31, 902-911 (2020). https:/doi.org:10.1016/j.annonc.2020.04.004
10 Forde, P. M. et al. Durvalumab with platinum-pemetrexed for unresectable pleural mesothelioma: survival, genomic and immunologic analyses from the phase 2 PrE0505 trial. Nat Med 27, 1910-1920 (2021). https://doi.org:10.1038/s41591-021-01541-0
11 Amemiya, H. M., Kundaje, A. & Boyle, A. P. The ENCODE Blacklist: Identification of Problematic Regions of the Genome. Sci Rep 9, 9354 (2019). https://doi.org:10.1038/841598-019-45839-z
12 Zook, J. M. et al. Integrating human sequence data sets provides a resource of benchmark SNP and indel genotype calls. Nat Biotechnol 32, 246-251 (2014). https://doi.org:10.1038/nbt.2835
13 Davoli, T., Uno, H., Wooten, E. C. & Elledge, S. J. Tumor aneuploidy correlates with markers of immune evasion and with reduced response to immunotherapy. Science 355 (2017). https://doi.org:10.1126/science.aaf8399
14 Anagnostou, V. et al. Integrative Tumor and Immune Cell Multi-omic Analyses Predict Response to Immune Checkpoint Blockade in Melanoma. Cell Rep Med 1, 100139 (2020). https://doi.org:10.1016/j.xcrm.2020.100139
15 Goodman, A. M. et al. MHC-I genotype and tumor mutational burden predict response to immunotherapy. Genome Med 12, 45 (2020). https://doi.org:10.1186/s13073-020-00743-4
16 Snyder, A. et al. Genetic basis for clinical response to CTLA-4 blockade in melanoma. N Engl J Med 371, 2189-2199 (2014). https://doi.org:10.1056/NEJMoa1406498
17 Rizvi, N. A. et al. Cancer immunology. Mutational landscape determines sensitivity to PD-1 blockade in non-small cell lung cancer. Science 348, 124-128 (2015). https://doi.org:10.1126/science.aaa1348
18 Samstein, R. M. et al. Tumor mutational load predicts survival after immunotherapy across multiple cancer types. Nat Genet 51, 202-206 (2019). https://doi.org:10.1038/s41588-018-0312-8
19 Vogelstein, B. et al. Cancer genome landscapes. Science 339, 1546-1558 (2013). https://doi.org:10.1126/science.1235122
20 Sha, D. et al. Tumor Mutational Burden as a Predictive Biomarker in Solid Tumors. Cancer Discov 10, 1808-1825 (2020). https://doi.org:10.1158/2159-8290.CD-20-0522
21 McGranahan, N. & Swanton, C. Neoantigen quality, not quantity. Science translational medicine 11 (2019). https://doi.org:10.1126/scitransimed.aax7918
22 Shao, X. M. et al. High-Throughput Prediction of MHC Class I and II Neoantigens with MHCnuggets. Cancer Immumol Res 8, 396-408 (2020). https://doi.org:10.1158/2326-6066 CIR-19-0464
23 Shao, X. M. et al. HLA class II immunogenic mutation burden predicts response to immune checkpoint blockade. Ann Oncol (2022). https://doi.org:10.1016/j.annonc.2022.03.013
24 Anagnostou, V. et al. Integrative tumor and immune cell mutli-omic analyses to predict melanoma response to immune checkpoint blockade. Cell Reports Medicine (2020)
25 Ellrott, K. et al. Scalable Open Science Approach for Mutation Calling of Tumor Exomes Using Multiple Genomic Pipelines. Cell Syst 6, 271-281 e277 (2018). https://doi.org:10.1016/j.cels.2018.03.002
26 Buckley, A. R. et al. Pan-cancer analysis reveals technical artifacts in TCGA germline variant calls. BMC Genomics 18, 458 (2017). https://doi.org:10.1186/s12864-017-3770-y
27 Taylor, A. M. et al. Genomic and Functional Approaches to Understanding Cancer Aneuploidy. Cancer Cell 33, 676-689 e673 (2018). https://doi. org: 10.1016/ccell 2018.03.007
28 Liu, J. et al. An Integrated TCGA Pan-Cancer Clinical Data Resource to Drive High-Quality Survival Outcome Analytics. Cell 173, 400-416 e411 (2018). https://doi.org:10.1016/j.cell.2018.02.052
29 Shen, R. & Seshan, V. E. FACETS: allele-specific copy number and clonal heterogeneity analysis tool for high-throughput DNA sequencing. Nucleic acids research 44, e131 (2016). https://doi.org:10.1093/nar/gkw520
30 Chen, S., Zhou, Y., Chen, Y. & Gu, J. fastp: an ultra-fast all-in-one FASTQ preprocessor. Bioinformatics 34, 1884-1890 (2018). https://doi.org:10.1093/bioinformatics/bty560
31 Langmead, B. & Salzberg, S. L. Fast gapped-read alignment with Bowtie 2. Nat Methods 9, 357-359 (2012). https://doi.org:10.1038/nmeth.1923
32 Tarasov, A., Vilella, A. J., Cuppen, E., Nijman, I. J. & Prins, P. Sambamba: fast processing of NGS alignment formats. Bioinformatics 31, 2032-2034 (2015). https://doi.org:10.1093/biomformatics/btv098
33 Saunders, C. T. et al. Strelka: accurate somatic small-variant calling from sequenced tumor-normal sample pairs. Bioinformatics 28, 1811-1817 (2012). https://doi.org:10.1093/bioinformatics/bts271
34 Kent, W. J. BLAT—the BLAST-like alignment tool. Genome Res 12, 656-664 (2002). https://doi.org:10.1101/gr.229202
35 Bonneville, R. et al. Landscape of Microsatellite Instability Across 39 Cancer Types. JCO precision oncology 2017 (2017). https://doi.org:10.1200/PO.17.00073
36 Niknafs, N., Beleva-Guthrie, V., Naiman, D. Q. & Karchin, R. SubClonal Hierarchy Inference from Somatic Mutations: Automatic Reconstruction of Cancer Evolutionary Trees from Multi-region Next Generation Sequencing. PLOS computational biology 11, e1004416 (2015). https://doi.org:10.1371/journal.pcbi.1004416
37 Hmeljak, J. et al. Integrative Molecular Characterization of Malignant Pleural Mesothelioma. Cancer Discov 8, 1548-1565 (2018). https://doi.org:10.1158/2159-8290.CD-18-0804
38 Bielski, C. M. et al. Genome doubling shapes the evolution and prognosis of advanced cancers. Nat Genet 50, 1189-1195 (2018). https://doi.org:10.1038/41588-018-0165-1
39 Riester, M. et al. PureCN: copy number calling and SNV classification using targeted short read sequencing Source Code Biol Med 11, 13 (2016). https://doi.org:10.1186/s13029-016-0060-z
40 Love, M. I., Huber, W. & Anders, S. Moderated estimation of fold change and dispersion for RNA-seq data with DESeq2. Genome Biol 15, 550 (2014). https://doi.org:10.1186/s13059-014-0550-8
41 Colaprico, A. et al. TCGAbiolinks: an R/Bioconductor package for integrative analysis of TCGA data. Nucleic Acids Res 44, e71 (2016). https://doi.org:10.1093/nar/gkv1807
42 Korotkevich, G., Sukhov, V. & Sergushichev, A. Fast gene set enrichment analysis. bioRxiv (2019). https://doi.org:https://doi.org/10.1101/060012
43 Thorsson, V. et al. The Immune Landscape of Cancer. Immunity 48, 812-830 e814 (2018). https://doi.org:10.1016/j.immuni.2018.03.023
44 Mandelker, D. et al. Navigating highly homologous genes in a molecular diagnostic setting: a resource for clinical next-generation sequencing. Genet Med 18, 1282-1289 (2016). https://doi.org:10.1038/gim.2016.58

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

1. A method of assessing and/or treating a subject diagnosed with a tumor, comprising
obtaining a biological sample from the subject;
identifying germline or somatic mutations in the subject's genome;
analyzing T cell receptor (TCR) clonotypes;
determining the number of mutations in haploid regions of the subject's genome; and
selecting the subject as being responsive to chemotherapy, immunotherapy or combined chemotherapy and immunotherapy treatment when the subject is determined to have a high number of mutations in haploid regions of the subject's genome.

2. A method of assessing and/or treating a subject diagnosed with a tumor, comprising
obtaining a biological sample from the subject;
identifying germline or somatic mutations in the subject's genome;
analyzing T cell receptor (TCR) clonotypes;
determining the number of mutations in polyploid regions of the subject's genome; and
selecting the subject as being responsive to chemotherapy, immunotherapy or combined chemotherapy and immunotherapy treatment when the subject is determined to have a high number of mutations in polyploid regions of the subject's genome.

3. (canceled)

4. The method of claim 1 further comprising administering cancer therapy to the subject selected as being responsive a chemotherapeutic, an immunotherapeutic or a chemotherapeutic and an immunotherapeutic, and thereby treating the subject.

5. (canceled)

6. The method of claim 1 wherein a high number of mutations in the haploid and/or polyploid regions of the genome is a 3, 5, 10, 15, 20, 25, 30, 35, 40, 45, 50, 60, 70, 80, 90 or 100 percent or more increased number of mutations in such regions relative to a subject that would not be responsive to chemotherapeutic and/or immunotherapeutic treatment.

7. The method of claim 1, wherein the tumor comprises a low tumor mutation burden (TMB-L), a medium tumor mutation burden (TMB-M) or a high tumor mutation burden (TMB-H).

8. The method of claim 1, wherein a high tumor mutational burden comprises 10 or more non-synonymous somatic mutations per megabase.

9-12. (canceled)

13. The method of claim 1 wherein the subject has non-small cell lung cancer, melanoma, an epithelioid tumor, a mesothelioma, and/or a malignant pleural mesothelioma (MPM).

14-17. (canceled)

18. The method of claim 1 wherein the nonsynonymous sequence alterations encode one or more immunogenic neoantigens.

19-21. (canceled)

22. The method of claim 1 wherein one or more germline mutations are detected in one or more cancer susceptibility genes.

23. The method of claim 22 wherein the one or more cancer susceptibility genes comprise BAP1, MLH1, MLH3, BRCA1/2, BLM or combinations thereof.

24. (canceled)

25. The method of claim 1 wherein the one or more chromatin genes comprise mutations in members of a SWI/SNF chromatin remodeling complex.

26. (canceled)

27. The method of claim 1 further comprising detecting mutations in genes KDM3B and KDM4C encoding histone demethylases and in KMT2C gene encoding methyltransferases.

28. The method of claim 1 wherein a subject is identified as having a high degree of tumor aneuploidy and genome-wide copy number breakpoints and thereby the subject is identified as responsive to chemotherapeutic and/or immunotherapeutic treatment.

29. The method of claim 1 wherein the subject is identified as having defective homologous recombination and thereby the subject is identified as responsive to chemotherapy and/or immunotherapy treatment.

30. The method of claim 1 wherein detection of mutation signatures indicative of an apolipoprotein B mRNA editing catalytic polypeptide-like (APOBEC) mutagenesis are predictive of non-responsiveness to chemotherapy and/or immunotherapy.

31-32. (canceled)

33. The method of claim 1 wherein an immunotherapeutic agent comprises: durvalumab, nivolumab, or atezolizuma and combinations thereof.

34. A method of assessing and/or treating a subject diagnosed with a tumor, comprising
obtaining a biological sample from the subject;
identifying germline or somatic mutations in the subject's genome;
analyzing T cell receptor (TCR) clonotypes; and
determining whether the subject has a high number of mutations in haploid regions of the genome;
identifying the subject that exhibits a high number of mutations in haploid regions of the genome as being responsive to chemotherapeutic and/or immunotherapeutic treatment; or

35-40. (canceled)

41. A method of identifying and distinguishing single-copy, multi-copy and persistent tumor mutations (pTMB) in a biological sample comprising:
performing a genome-wide analysis of sequence coverage distribution and b-allele frequency of heterozygous single nucleotide polymorphisms (SNPs) for determining purity and segmental tumor and normal copy numbers;
calculating the expected variant allele fraction for a mutation at a cellular fraction with mutant copies per cancer cell and calculating mutation clonality for each cancer cell in the biological sample;
assigning minor and major copy numbers to mutated loci and classifying mutations as single-copy, multi-copy or persistent tumor mutations; and
identifying and distinguishing single-copy, multi-copy and persistent tumor mutations.

42-47. (canceled)

48. The method of claim 1 wherein the number of mutations in haploid and polyploid regions of the subject's genome are determined.
